SEQUENCE LISTING (56 sequences)

SEQ ID NO: 1 - RNA, 36 nt, Artificial Sequence
  Synthetic CCR5
  misc_feature: AltR
gcuguguuugcgucucucccguuuuagagcuaugcu

SEQ ID NO: 2 - RNA, 36 nt, Artificial Sequence; identical to SEQ ID NO: 1
  Synthetic HPRT
  misc_feature: AltR

SEQ ID NO: 3 - RNA, 36 nt, Artificial Sequence
  Synthetic HBB
  misc_feature: AltR
cuugccccacagggcaguaaguuuuagagcuaugcu

SEQ ID NO: 4 - RNA, 36 nt, Artificial Sequence
  Synthetic TCR
  misc_feature: AltR
gagaaucaaaaucggugaauguuuuagagcuaugcu

SEQ ID NO: 5 - RNA, 36 nt, Artificial Sequence
  Synthetic PD-1
  misc_feature: AltR
ugacguuaccucgugcggccguuuuagagcuaugcu

SEQ ID NO: 6 - RNA, 36 nt, Artificial Sequence
  Synthetic BCL11a
  misc_feature: AltR
gauaaacaaucgucauccucguuuuagagcuaugcu

SEQ ID NO: 7 - RNA, 36 nt, Artificial Sequence
  Synthetic C9orf72
  misc_feature: AltR
uacuacaacggaacagccacguuuuagagcuaugcu

SEQ ID NO: 8 - RNA, 100 nt, Artificial Sequence
  Synthetic gDrosha1-full
  misc_feature: contains 2-O-Me and PS bonds
ugcugcggcaugacuggaggguuuuagagcuagaaauagcaaguuaaaauaaggcuaguccguuaucaacuugaaaaaguggcaccgagucggugcuuuu

SEQ ID NO: 9 - RNA, 100 nt, Artificial Sequence
  Synthetic gDrosha2-full
  misc_feature: contains 2-O-Me and PS bonds
ugagugcaucgaauccacacguuuuagagcuagaaauagcaaguuaaaauaaggcuaguccguuaucaacuugaaaaaguggcaccgagucggugcuuuu

SEQ ID NO: 10 - RNA, 100 nt, Artificial Sequence
  Synthetic gHPRT4-full
  misc_feature: contains 2-O-Me and PS bonds
gcgggucgccauaacggagcguuuuagagcuagaaauagcaaguuaaaauaaggcuaguccguuaucaacuugaaaaaguggcaccgagucggugcuuuu

SEQ ID NO: 11 - RNA, 100 nt, Artificial Sequence
  Synthetic gHPRT3-full
  misc_feature: contains 2-O-Me and PS bonds
ggcuuauauccaacacuucgguuuuagagcuagaaauagcaaguuaaaauaaggcuaguccguuaucaacuugaaaaaguggcaccgagucggugcuuuu

SEQ ID NO: 12 - RNA, 100 nt, Artificial Sequence
  Synthetic gWas1
  misc_feature: contains 2-O-Me and PS bonds
cgccaugggucauuucacagguuuuagagcuagaaauagcaaguuaaaauaaggcuaguccguuaucaacuugaaaaaguggcaccgagucggugcuuuu

SEQ ID NO: 13 - RNA, 100 nt, Artificial Sequence
  Synthetic gHBG1-117
  misc_feature: contains 2-O-Me and PS bonds
cuugucaaggcuauuggucaguuuuagagcuagaaauagcaaguuaaaauaaggcuaguccguuaucaacuugaaaaaguggcaccgagucggugcuuuu

SEQ ID NO: 14 - RNA, 100 nt, Artificial Sequence
  Synthetic gHBG2-114
  misc_feature: contains 2-O-Me and PS bonds
guuugccuugucaaggcuauguuuuagagcuagaaauagcaaguuaaaauaaggcuaguccguuaucaacuugaaaaaguggcaccgagucggugcuuuu

SEQ ID NO: 15 - RNA, 100 nt, Artificial Sequence
  Synthetic gHBB2
  misc_feature: contains 2-O-Me and PS bonds
guaacggcagacuucuccucguuuuagagcuagaaauagcaaguuaaaauaaggcuaguccguuaucaacuugaaaaaguggcaccgagucggugcuuuu

SEQ ID NO: 16 - RNA, 44 nt, Artificial Sequence
  Synthetic gHBG12a1
uaauuucuacucuuguagauccuugucaaggcuauuggucaagg

SEQ ID NO: 17 - RNA, 44 nt, Artificial Sequence
  Synthetic gHBG12a2
uaauuucuacucuuguagaugccagggaccguuucagacagaua

SEQ ID NO: 18 - RNA, 44 nt, Artificial Sequence
  Synthetic gHBG12a3
uaauuucuacucuuguagauagacagauauuugcauugagauag

SEQ ID NO: 19 - RNA, 44 nt, Artificial Sequence
  Synthetic gHBG12a4
uaauuucuacucuuguagaucauugagauaguguggggaagggg

SEQ ID NO: 20 - RNA, 44 nt, Artificial Sequence
  Synthetic gHBB12a1
uaauuucuacucuuguagaucuucugacacaacuguguucacua

SEQ ID NO: 21 - RNA, 44 nt, Artificial Sequence
  Synthetic gHBB12a2
uaauuucuacucuuguagauagguugcuagugaacacaguugug

SEQ ID NO: 22 - RNA, 44 nt, Artificial Sequence
  Synthetic gHPRT12a1
uaauuucuacucuuguagauagaucuuacuuaccuguccauaau

SEQ ID NO: 23 - RNA, 44 nt, Artificial Sequence
  Synthetic gHPRT12a2
uaauuucuacucuuguagaugcauaccuaaucauuaugcugagg

SEQ ID NO: 24 - DNA, 23 bp, Artificial Sequence
  Synthetic Gamma-globin promoter
cttgtcaaggctattggtcaagg

SEQ ID NO: 25 - DNA, 23 bp, Artificial Sequence
  Synthetic Gamma-globin promoter
caaggctattggtcaaggcaagg

SEQ ID NO: 26 - DNA, 23 bp, Artificial Sequence
  Synthetic Gamma-globin promoter
gctattggtcaaggcaaggctgg

SEQ ID NO: 27 - DNA, 23 bp, Artificial Sequence
  Synthetic Gamma-globin promoter
tggtcaagtttgccttgtcaagg

SEQ ID NO: 28 - DNA, 23 bp, Artificial Sequence
  Synthetic Gamma-globin promoter
cttgaccaatagccttgacaagg

SEQ ID NO: 29 - DNA, 28 bp, Artificial Sequence
  Synthetic Gamma-globin promoter
tttgccttgtcaaggctattggtcaagg

SEQ ID NO: 30 - DNA, 20 bp, Artificial Sequence
  Synthetic HPRT
gttatggcgacccgcagccc

SEQ ID NO: 31 - DNA, 20 bp, Artificial Sequence
  Synthetic HPRT
gcgggtcgccataacggagc

SEQ ID NO: 32 - DNA, 20 bp, Artificial Sequence
  Synthetic HPRT
cgtgacgtaaagccgaaccc

SEQ ID NO: 33 - DNA, 20 bp, Artificial Sequence
  Synthetic HPRT
ggcacggaaagcgaccacct

SEQ ID NO: 34 - DNA, 20 bp, Artificial Sequence
  Synthetic HPRT
catgcgtatttgacacacga

SEQ ID NO: 35 - DNA, 20 bp, Artificial Sequence
  Synthetic HPRT
gctaagtgctagagttacgg

SEQ ID NO: 36 - DNA, 20 bp, Artificial Sequence
  Synthetic HPRT
gaggcgcgggatccgcagtg

SEQ ID NO: 37 - DNA, 20 bp, Artificial Sequence
  Synthetic HPRT
ttattactgttccccgccag

SEQ ID NO: 38 - DNA, 20 bp, Artificial Sequence
  Synthetic
ttactgccctgtggggcaag

SEQ ID NO: 39 - DNA, 52 bp, Artificial Sequence
  Synthetic
gactcctgaggagaagtctgccgttactgccctgtggggcaaggtgaacgtg

SEQ ID NO: 40 - DNA, 50 bp, Artificial Sequence
  Synthetic
gactcctgaggagaagtctgccgttatgcccttggggcaaggtgaacgtg

SEQ ID NO: 41 - DNA, 50 bp, Artificial Sequence
  Synthetic
gactcctgaggagaagtctgccgttagccctgtggggcaaggtgaacgtg

SEQ ID NO: 42 - DNA, 49 bp, Artificial Sequence
  Synthetic
gactcctgaggagaagtctgccgttaccctgtggggcaaggtgaacgtg

SEQ ID NO: 43 - DNA, 47 bp, Artificial Sequence
  Synthetic
gactcctgaggagaagtctgccgtacctgtggggcaaggtgaacgtg

SEQ ID NO: 44 - DNA, 42 bp, Artificial Sequence
  Synthetic
gactcctgaggagaagtctgccgttggggcaaggtgaacgtg

SEQ ID NO: 45 - DNA, 52 bp, Artificial Sequence
  Synthetic
gactcctgtggagaagtctgccgttactgccctgtggggcaaggtgaacgtg

SEQ ID NO: 46 - DNA, 52 bp, Artificial Sequence; identical to SEQ ID NO: 45
  Synthetic

SEQ ID NO: 47 - DNA, 52 bp, Artificial Sequence; identical to SEQ ID NO: 45
  Synthetic

SEQ ID NO: 48 - DNA, 52 bp, Artificial Sequence; identical to SEQ ID NO: 39
  Synthetic

SEQ ID NO: 49 - DNA, 50 bp, Artificial Sequence
  Synthetic
gactcctgaggagaagtctgccgttacccctgtggggcaaggtgaacgtg

SEQ ID NO: 50 - DNA, 49 bp, Artificial Sequence
  Synthetic
gactcctgaggagaagtctgccgttgccctgtggggcaaggtgaacgtg

SEQ ID NO: 51 - DNA, 48 bp, Artificial Sequence
  Synthetic
gactcctgaggagaagtctgccgtgccctgtggggcaaggtgaacgtg

SEQ ID NO: 52 - DNA, 48 bp, Artificial Sequence; identical to SEQ ID NO: 51
  Synthetic

SEQ ID NO: 53 - DNA, 46 bp, Artificial Sequence
  Synthetic
gactcctgaggagaagtctgccgccctgtggggcaaggtgaacgtg

SEQ ID NO: 54 - DNA, 52 bp, Artificial Sequence; identical to SEQ ID NO: 45
  Synthetic

SEQ ID NO: 55 - DNA, 630 bp, Artificial Sequence
  Synthetic
gctttaaaagccaggacggtcacctttggggtggtgacaagtgtgatcacttgggtggtggctgtgtttgcgtctggatccggagctactaatttctccttgcttaagcaagctggtgatgttgaagaaaatcctggtcctatgggagcaggagcaaccggaagggcaatggacggaccaagattgttacttctgctcctgctaggcgtgagcctgggaggagcaaggagctggatggagtgggacagggagatcaacaactacaccagcctgatccacagcctgatcgaggagagccagaaccagcaggagaagaacgagcaggagctgctggagctggacaagtgggccagcctgtggaactggttccggagcgagcggaagtgctgcgtggagtgcccaccatgcccagcaccaccagtggcaggacccctgatcgcactggtgaccagcggagccctgctggccgtgctgggcatcacaggctacttcctgatgaacaggaggagctggagcccaaccggagagcggctggagctggagccatgaaagcttaggaatcatctttaccagatctcaaaaagaaggtcttcattacacctgcagctctcattttccatacagtcagta

SEQ ID NO: 56 - DNA, 100 bp, Artificial Sequence
  Synthetic
cactagcaacctcaaacagacaccatggtgcatctgactcctgtggagaagtctgccgttactgccctgtggggcaaggtgaacgtggatgaagttggtg

(19) United States
(12) Patent Application Publication
(10) Pub. No.: US 2021/0322331 A1
(43) Pub. Date: Oct. 21, 2021

(54) NANOCAPSULES FOR THE DELIVERY OF CELL MODULATING AGENTS

(71) Applicant: CSL BEHRING LLC, King of Prussia, PA (US)

(72) Inventors: Ming Yan, Encino, CA (US); Jeffrey S. Bartlett, Columbus, OH (US)

(21) Appl. No.: 17/353,440

(22) Filed: Jun. 21, 2021

Related U.S. Application Data
(63) Continuation of application No. PCT/US2019/068259, filed on Dec. 23, 2019.
(60) Provisional application No. 62/784,503, filed on Dec. 23, 2018.

(51) Int. Cl. A61K 9/51; A61K 48/00; C12N 9/22; C12N 15/11; A61K 47/69; A61K 47/68; C12N 5/0789; A61K 35/28

(52) U.S. Cl. CPC A61K 9/5138; A61K 48/0041; C12N 9/22; C12N 15/11; A61K 47/6925; C12N 2510/00; C12N 5/0647; A61K 35/28; C12N 2310/20; C12N 2320/32; C12N 2800/80; A61K 47/6849

(57) ABSTRACT

The present disclosure is directed to polymer nanocapsules including a ribonucleoprotein complex, and methods of their delivery. In some embodiments, the polymer nanocapsule comprises a polymer shell and a ribonucleoprotein complex, wherein the polymer shell comprises at least one positively charged monomer, at least one neutral monomer, and a crosslinker. Also disclosed are conjugates of polymer nanocapsules coupled to one or more targeting moieties and/or one or more stabilizing moieties.

### CROSS REFERENCE TO RELATED APPLICATIONS

The present disclosure is a continuation of International Application No. PCT/US2019/068259 filed on Dec. 23, 2019, which application claims the benefit of the filing date of U.S. Provisional Application No. 62/784,503, filed on Dec. 23, 2018, the disclosures of which are hereby incorporated by reference herein in their entireties.

### FIELD OF DISCLOSURE

The present disclosure generally relates to gene therapy. More specifically, the present disclosure relates to a nanocapsule including a ribonucleoprotein complex and their application in gene therapy. In particular, the present disclosure relates to a nanocapsule including a ribonucleoprotein complex their application in vivo and/or ex vivo, including in hematopoietic stem cells.

### BACKGROUND OF THE DISCLOSURE

Technologies enabling the precise modification of DNA sequences within living cells are valuable for both basic and applied research. Clustered Regularly Interspaced Short Palindromic Repeats (CRISPR) are prokaryotic immune systems first discovered by Ishino in E. coli. (Ishino, et al., Journal of Bacteriology 169(12): 5429-5433 (1987)). These systems provide immunity in bacteria and archaea against viruses and plasmids by targeting the nucleic acids of the viruses and plasmids in a sequence-specific manner.

It is believed that there are two main stages involved in providing the aforementioned immunity, namely an acquisition stage and an interference stage. The acquisition stage involves cutting the genome of invading viruses and plasmids and integrating segments of this into the CRISPR locus of the bacteria and archaea. The segments to be integrated into the genome are known as protospacers and help in protecting the organism from subsequent attack by the same virus or plasmid. The interference stage involves attacking an invading virus or plasmid. It is believed that this particular stage relies upon the integrated sequences, called spacers, being transcribed into RNA and, following some processing, this RNA then hybridizes with a complementary sequence in the DNA or RNA of an invading polynucleotide (e.g., a virus or a plasmid) while also associating with a protein, or protein complex, that effectively binds and/or cleaves the DNA or RNA.

There are several different CRISPR-Cas systems. In Class 2 Type II systems there are two strands of RNA that are part of the CRISPR-Cas system: a CRISPR RNA (crRNA) and a transactivating CRISPR RNA (tracrRNA). The tracrRNA hybridizes to a complementary region of pre-crRNA facilitating maturation of the pre-crRNA to crRNA by an RNase III enzyme. The duplex formed by the tracrRNA and crRNA is recognized by, and associates with a protein, for example Cas9, which is directed to a target nucleic acid by a sequence of the crRNA that is complementary to, and hybridizes with, a sequence in the target nucleic acid. It is believed that these minimal components of the RNA-based immune system can be reprogrammed to target DNA in a site-specific manner by using a single protein and two RNA guide polynucleotides or a single RNA molecule.

In Class 2 Type V CRISPR systems it is believed that the Cas protein Cpf1 can be reprogrammed to target DNA in a site-specific manner with a single crRNA sequence.

Overall, the expression of the bacterial Cas9 nuclease, as well as a short stretch of RNA containing genomic targeting information (e.g., a 20 bp sequence) and a structural component to associate with Cas9 itself, allow for the precise placement of a double stranded or single stranded break (DSB or SSB) at a desired location(s) within a genome of interest. The CRISPR-Cas system is believed to be superior to other methods of genome editing, such as endonucleases, meganucleases, zinc finger nucleases, and transcription activator-like effector nucleases (TALENs), which may require de novo protein engineering for every new target locus.

Gene editing techniques such as the CRISPR/Cas9 system, have been shown to work well in a variety of cell lines as well as for germline genome editing (see Doudna et al., Genome editing, The new frontier of genome engineering with CRISPR-Cas9, Science 346(6213):1258096). Lentiviral vectors are reasonably flexible to allow targeting of genomic loci and they show great potential for the purpose of screening of guide-RNA (gRNA) for CRISPR system, but integration of Cas9 has been shown to have limited potential therapeutic use (Hsu et al., Development and applications of CRISPR-Cas9 for genome engineering. Cell 157(6):1262-1278).

Several groups have developed adeno-associated viral vectors (AAV) for in vivo gene editing due to their low immunogenicity and broad tropism. However, packaging capacity and the need for very high titer for delivery to primary cells are major concerns (see Ran et al., “In vivo genome editing using Staphylococcus aureus Cas9,” Nature. 2015 Apr. 9; 520 (7546); 186-91; Swiech et al., “In vivo interrogation of gene function in the mammalian brain using CRISPR-Cas9,” Nat Biotechnol. 2015 January; 33(1):102-6; and Cong et al., “Multiplex genome engineering using CRISPR/Cas systems,” Science, 2013 Feb. 15; 339(6121):819-23).

Plasmid delivery of Cas9 and single-guide RNAs (sgRNAs) has been shown to be efficient in other cell types but ablated only 1-5% of target protein expression in human primary T cells and hematopoietic stem cells (see Mandal et al., “Efficient ablation of genes in human hematopoietic stem and effector cells using CRISPR/Cas9,” Stem Cell 15(5):643-652). Electroporation of Cas9 ribonucleoproteins demonstrated efficient and specific genome editing in T cells and stem cells but there exist technical barriers to applying electroporation to an in vivo study (see Schumann et al., “Generation of knock-in primary human T cells using Cas9 ribonucleoproteins,” Proc Natd Acad Sci, 2015 Aug. 18; 112(33):10437-42). Thus, to date, in vivo delivery of gene editing factors still presents a challenge.

### BRIEF SUMMARY OF THE DISCLOSURE

In a first aspect of the present disclosure is a polymer nanocapsule comprising a polymer shell and a ribonucleoprotein complex, wherein the polymer shell comprises at least one positively charged monomer, at least one neutral monomer, and a crosslinker. In some embodiments, the polymer shell comprises at least two positively charged monomers. In some embodiments, ribonucleoprotein complex comprises an endonuclease and a guide RNA. In some embodiments, the endonuclease is a Cas protein. In some embodiments, the Cas protein is selected from the group consisting of Cas9, Cas12a, and Cas12b.

In some embodiments, the guide RNA targets the HPRT locus. In some embodiments, the guide RNA that targets HPRT comprises a nucleic acid sequence having at least 90% identity to that of SEQ ID NO: 2. In some embodiments, the guide RNA that targets HPRT comprises a nucleic acid sequence having at least 95% identity to that of SEQ ID NO: 2. In some embodiments, the guide RNA targets the beta-globin locus. In some embodiments, the guide RNA that targets beta-globin comprises a nucleic acid sequence having at least 90% identity to that of SEQ ID NO: 3.

In some embodiments, the guide RNA that targets beta-globin comprises a nucleic acid sequence having at least 95% identity to that of SEQ ID NO: 3. In some embodiments, the guide RNA has at least 90% identity to any one of SEQ ID NOS: 39-54. In some embodiments, the guide RNA has at least 95% identity to any one of SEQ ID NOS: 39-54. In some embodiments, the guide RNA comprises any one of SEQ ID NOS: 39-54. In some embodiments, the guide RNA has at least 90% identity to any one of SEQ ID NOS: 1 and 4-23. In some embodiments, the guide RNA has at least 95% identity to any one of SEQ ID NOS: 1 and 4-23. In some embodiments, the guide RNA comprises any one of SEQ ID NOS: 1 and 4-23.

In some embodiments, the at least one positively charged monomer is selected from the group consisting of:
N-(3-((4-((3-aminopropyl)amino)butyl)amino) propyl)acrylamide,
N-(3-((4-((3-aminopropyl)amino)butyl)amino)propyl)methacrylamide,
N-(3-((4-aminobutyl)amino)propyl)acrylamide,
N-(3-((4-aminobutyl)amino)propyl)methacrylamide,
N-(2-((2-aminoethyl)(methyl)amino)ethyl)acrylamide,
N-(2-((2-aminoethyl)(methyl)amino)ethyl)methacrylamide,
N-(piperazin-1-ylmethyl)acrylamide,
N-(piperazin-1-ylmethyl)methacrylamide,
N-(2-(bis(2-aminoethyl)amino)ethyl)acrylamide,
N-(2-(bis(2-aminoethyl)amino)ethyl)methacrylamide,
N-(3-Aminopropyl)methacrylamidehydrochloride,
2-(dimethylamino)ethyl acrylate,
Dimethylamino ethyl methacrylate,
(3-Acrylamidopropyl) trimethylammonium hydrochloride,
2-aminoethyl methacrylate,
3-(Dimethylamino)propyl acrylate,
N-[3-(Dimethylamino)propyl]methacrylamide, and any combination thereof.

In some embodiments, the neutral monomer is selected from N-(1,3-dihydroxy-2-(hydroxymethyl)propan-2-yl)acrylamide, acrylamide, N-(hydroxymethyl)acrylamide, 2-hydroxyethyl acrylate, and 2-hydroxyethyl methacrylate. In some embodiments, the crosslinkers are selected from the group consisting of 1,3-glycerol dimethacrylate, N,N′-Methylenebisacrylamide, and glycerol 1,3-diglycerolate diacrylate.

In some embodiments, the polymer nanocapsule further comprises at least one targeting moiety. In some embodiments, the polymer nanocapsule comprises between 2 and between 6 targeting moieties. In some embodiments, the at least one targeting moiety is an antibody. In some embodiments, the at least one targeting moiety is an antibody and wherein the polymer nanocapsule comprises between 1 and 3 antibodies. In some embodiments, the polymer nanocapsule comprises at least one stabilizing moiety. In some embodiments, the at least one stabilizing moiety comprises at least one polyethylene glycol group. In some embodiments the polymer nanocapsule comprises at least one targeting moiety and at least one stabilizing moiety. In some embodiments, the polymer nanocapsule comprises between 2 and 6 targeting moieties and between at least 2 and 6 stabilizing moieties. In some embodiments, the at least one targeting moiety is an antibody, and the stabilizing moiety comprises at least one polyethylene glycol group.

In some embodiments, the polymer shell is free from monomers or crosslinkers including a heterocyclic group. In some embodiments, the polymer shell is free from monomers or crosslinkers including an imidazole group. In some embodiments, the polymer shell is free of imidazolyl acryloyl monomers.

In second aspect of the present disclosure is a composition comprising: (i) a polymer nanocapsule including a polymer shell and a ribonucleoprotein complex, wherein the polymer shell comprises at least one positively charged monomer, at least one neutral monomer, and a crosslinker, and (ii) a pharmaceutically acceptable carrier or excipient. In some embodiments, ribonucleoprotein complex comprises an endonuclease and a guide RNA. In some embodiments, the endonuclease is a Cas protein. In some embodiments, the Cas protein is selected from the group consisting of Cas9, Cas12a, and Cas12b. In some embodiments, the polymer shell comprises at least two positively charged monomers.

In some embodiments, the guide RNA targets the HPRT locus. In some embodiments, the guide RNA that targets HPRT comprises a nucleic acid sequence having at least 90% identity to that of SEQ ID NO: 2. In some embodiments, the guide RNA that targets HPRT comprises a nucleic acid sequence having at least 95% identity to that of SEQ ID NO: 2. In some embodiments, the guide RNA targets the beta-globin locus. In some embodiments, the guide RNA that targets beta-globin comprises a nucleic acid sequence having at least 90% identity to that of SEQ ID NO: 3. In some embodiments, the guide RNA that targets beta-globin comprises a nucleic acid sequence having at least 95% identity to that of SEQ ID NO: 3.

In some embodiments, the guide RNA has at least 90% identity to any one of SEQ ID NOS: 39-54. In some embodiments, the guide RNA has at least 95% identity to any one of SEQ ID NOS: 39-54. In some embodiments, the guide RNA comprises any one of SEQ ID NOS: 39-54. In some embodiments, the guide RNA has at least 90% identity to any one of SEQ ID NOS: 1 and 4-23. In some embodiments, the guide RNA has at least 95% identity to any one of SEQ ID NOS: 1 and 4-23. In some embodiments, the guide RNA comprises any one of SEQ ID NOS: 1 and 4-23.

In some embodiments, the polymer shell is free from monomers or crosslinkers including a heterocyclic group. In some embodiments, the polymer shell is free from monomers or crosslinkers including an imidazole group. In some embodiments, the polymer shell is free of imidazolyl acryloyl monomers.

In a third aspect of the present disclosure is a modified host cell prepared by: contacting a host cell with one or more polymer nanocapsules, wherein the one or more polymer nanocapsules comprise a polymer shell and a ribonucleoprotein complex, and wherein the polymer shell comprises at least one positively charged monomer, at least one neutral monomer, and a crosslinker. In some embodiments, the polymer shell comprises at least two positively charged monomers. In some embodiments, the host cell is a hematopoietic stem cell. In some embodiments, the hematopoietic stem cell is an allogenic hematopoietic stem cell. In some embodiments, the hematopoietic stem cell is an autologous hematopoietic stem cell. In some embodiments, the hematopoietic stem cell is a sibling matched hematopoietic stem cell.

In some embodiments, ribonucleoprotein complex comprises an endonuclease and a guide RNA. In some embodiments, the endonuclease is a Cas protein. In some embodiments, the Cas protein is selected from the group consisting of Cas9, Cas12a, and Cas12b. In some embodiments, the guide RNA targets the HPRT locus. In some embodiments, the guide RNA that targets HPRT comprises a nucleic acid sequence having at least 90% identity to that of SEQ ID NO: 2. In some embodiments, the guide RNA that targets HPRT comprises a nucleic acid sequence having at least 95% identity to that of SEQ ID NO: 2. In some embodiments, the guide RNA targets the beta-globin locus. In some embodiments, the guide RNA that targets beta-globin comprises a nucleic acid sequence having at least 90% identity to that of SEQ ID NO: 3.

In some embodiments, the guide RNA that targets beta-globin comprises a nucleic acid sequence having at least 95% identity to that of SEQ ID NO: 3. In some embodiments, the guide RNA has at least 90% identity to any one of SEQ ID NOS: 39-54. In some embodiments, the guide RNA has at least 95% identity to any one of SEQ ID NOS: 39-54. In some embodiments, the guide RNA comprises any one of SEQ ID NOS: 39-54. In some embodiments, the guide RNA has at least 90% identity to any one of SEQ ID NOS: 1 and 4-23. In some embodiments, the guide RNA has at least 95% identity to any one of SEQ ID NOS: 1 and 4-23. In some embodiments, the guide RNA comprises any one of SEQ ID NOS: 1 and 4-23.

In some embodiments, the polymer nanocapsule is free from monomers or crosslinkers including a heterocyclic group. In some embodiments, the polymer nanocapsule is free from monomers or crosslinkers including an imidazole group.

In a fourth aspect of the present disclosure is a conjugate prepared according to a process comprising: (i) derivatizing a polymer nanocapsule with a first reactive functional group capable of participating in a click chemistry reaction, the polymer nanocapsule having a polymer shell and a ribonucleoprotein complex, wherein the polymer shell comprises at least one positively charged monomer, at least one neutral monomer, and a crosslinker; (ii) derivatizing a targeting moiety with a second reactive functional group capable of participating in a click chemistry reaction with the first reactive functional group; and (iii) reacting the derivatized polymer nanocapsule with the derivatized targeting moiety.

In some embodiments, the polymer shell comprises at least two different positively charged monomers. In some embodiments, the ribonucleoprotein complex comprises a Cas protein and a guide RNA. In some embodiments, the guide RNA targets the HPRT locus. In some embodiments, the guide RNA that targets HPRT comprises a nucleic acid sequence having at least 90% identity to that of SEQ ID NO: 2. In some embodiments, the guide RNA targets the beta-globin locus.

In some embodiments, the guide RNA that targets beta-globin comprises a nucleic acid sequence having at least 90% identity to that of SEQ ID NO: 3. In some embodiments, the guide RNA has at least 90% identity to any one of SEQ ID NOS: 39-54. In some embodiments, the guide RNA has at least 95% identity to any one of SEQ ID NOS: 39-54. In some embodiments, the guide RNA comprises any one of SEQ ID NOS: 39-54. In some embodiments, the guide RNA has at least 90% identity to any one of SEQ ID NOS: 1 and 4-23. In some embodiments, the guide RNA has at least 95% identity to any one of SEQ ID NOS: 1 and 4-23. In some embodiments, the guide RNA comprises any one of SEQ ID NOS: 1 and 4-23.

In some embodiments, the polymer shell is free from monomers or crosslinkers including a heterocyclic group. In some embodiments, the polymer shell is free from monomers or crosslinkers including an imidazole group. In some embodiments, the polymer shell is free of imidazolyl acryloyl monomers.

In some embodiments, the targeting moiety is derivatized by reacting the targeting moiety with a compound of Formula (IIIA):

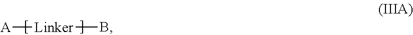

wherein

A is maleimide-C(O)—;

“Linker” is a branched or unbranched, linear or cyclic, substituted or unsubstituted, saturated or unsaturated, group having between 2 and 40 carbon atoms, and optionally having one or more heteroatoms selected from O, N, or S; and

B is the second reactive functional group selected from the group consisting of dibenzocyclooctyne (“DBCO”), trans-cyclooctene (“TCO”), azide, tetrazine, maleimide, thiol, 1,3-nitrone, aldehyde, ketone, hydrazine, and hydroxylamine.

In some embodiments, the “Linker” has the structure of Formula (IIIB):

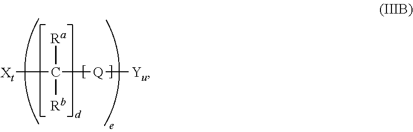

wherein d and e each independently are integers ranging from 2 to 10; t and u are independently 0 or 1; Q is a bond, O, S, or N(R^{c})(R^{d}); R^{a} and R^{b} are independently H, a C₁-C₄ alkyl group, or a halogen; R^{c} and R^{d} are independently CH₃ or H; and X and Y are independently a branched or unbranched, saturated or unsaturated group having between 1 and 4 carbon atoms and optionally having one or more O, N, or S heteroatoms.

In some embodiments, the derivatized polymer nanocapsule is further reacted with a stabilizing moiety of Formula (V):

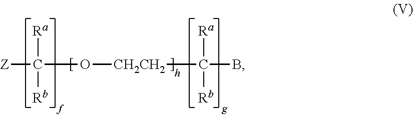

wherein B is the second reactive functional group selected from the group consisting of dibenzocyclooctyne (“DBCO”), trans-cyclooctene (“TCO”), azide, tetrazine, maleimide, thiol, 1,3-nitrone, aldehyde, ketone, hydrazine, and hydroxylamine;

Z is a hydroxyl group, a branched or unbranched C₁-C₄ alkyl group, —O-alkyl, or —NH2;

f and g are independently 0 or an integer ranging from 1 to 4; and

h is an integer ranging from 1 to 24.

In some embodiments, the polymer nanocapsule is derivatized with at least three of the first reactive functional groups. In some embodiments, the first reactive group is one of DBCO, TCO, maleimide, an aldehyde, a ketone, an azide, a tetrazine, a thiol, a 1,3-nitrone, a hydrazine, and hydroxylamine. In some embodiments, the first reactive functional group is a DBCO group and the second reactive functional group is an azide group.

In some embodiments, the targeting moiety is an antibody. In some embodiments, the antibody is selected from the group consisting of an anti-CD4 antibody, an anti-CD8 antibody, and an anti-CD45 antibody. In some embodiments, the conjugate is targeted to a cell having a cluster of differentiation marker selected from CD3, CD4, CD8, or CD45.

In some embodiments, the polymer nanocapsule is derivatized with the first reactive functional group by reacting the polymer nanocapsule with a compound comprising a disulfide group. In some embodiments, the polymer nanocapsule is derivatized with the first reactive functional group by reacting the polymer nanocapsule with a compound of Formula (IVA):

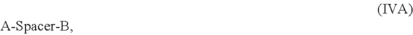

wherein A is maleimide-C(O)—,

“Spacer” is a branched or unbranched, linear or cyclic, substituted or unsubstituted, saturated or unsaturated group having between 2 and 20 carbon atoms, and having a disulfide bond; and

B is selected from the group consisting of dibenzocyclooctyne (“DBCO”), trans-cyclooctene (“TCO”), azide, tetrazine, maleimide, thiol, 1,3-nitrone, aldehyde, ketone, hydrazine, and hydroxylamine.

In some embodiments, the ‘Spacer’ has the structure of Formula (IVB

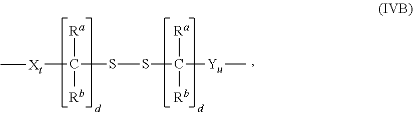

wherein

d is an integer ranging from 2 to 10; t and u are independently 0 or 1; Ra and Rb are independently H, a C₁-C₄ alkyl group, or a halogen; and X and Y are independently a branched or unbranched, saturated or unsaturated group having between 1 and 8 carbon atoms and optionally having one or more O, N, or S heteroatoms.

In a fifth aspect of the present disclosure is a composition comprising: (a) a conjugate, wherein the conjugate is prepared according to a process comprising: (i) derivatizing a polymer nanocapsule with a first reactive functional group capable of participating in a click chemistry reaction, the polymer nanocapsule having a polymer shell and a ribonucleoprotein complex, wherein the polymer shell comprises at least one positively charged monomer, at least one neutral monomer, and a crosslinker; (ii) derivatizing a targeting moiety with a second reactive functional group capable of participating in a click chemistry reaction with the first reactive functional group; and, (iii) reacting the derivatized polymer nanocapsule with the derivatized targeting moiety; and (b) a pharmaceutically acceptable carrier or excipient. In some embodiments, the polymer shell comprises at least two positively charged monomers. In some embodiments, the conjugate comprises between 2 and 6 targeting moieties. In some embodiments, ribonucleoprotein complex comprises an endonuclease and a guide RNA. In some embodiments, the endonuclease is a Cas protein. In some embodiments, the Cas protein is selected from the group consisting of Cas9, Cas12a, and Cas12b. In some embodiments, the polymer shell is free from monomers or crosslinkers including a heterocyclic group. In some embodiments, the polymer shell is free from monomers or crosslinkers including an imidazole group. In some embodiments, the polymer shell is free of imidazolyl acryloyl monomers.

In a sixth aspect of the present disclosure is a modified host cell prepared by contacting a host cell with either (i) a polymer nanocapsule comprising a polymer shell and a ribonucleoprotein complex, wherein the polymer shell comprises at least one different positively charged monomer, at least one neutral monomer, and a crosslinker; or (ii) a polymer nanocapsule conjugate, wherein the conjugate comprises (a) a polymer shell and a ribonucleoprotein complex, wherein the polymer shell comprises at least two different positively charged monomers, at least one neutral monomer, and a crosslinker; and (b) one or more targeting moieties and/or one or more stabilizing moieties. In some embodiments, the polymer shell comprises at least two positively charged monomers. In some embodiments, the host cell is a hematopoietic stem cell. In some embodiments, the hematopoietic stem cell is an allogenic hematopoietic stem cell. In some embodiments, the hematopoietic stem cell is an autologous hematopoietic stem cell. In some embodiments, the hematopoietic stem cell is a sibling matched hematopoietic stem cell.

In some embodiments, ribonucleoprotein complex comprises an endonuclease and a guide RNA. In some embodiments, the endonuclease is a Cas protein. In some embodiments, the Cas protein is selected from the group consisting of Cas9, Cas12a, and Cas12b. In some embodiments, the guide RNA targets the HPRT locus. In some embodiments, the guide RNA that targets HPRT comprises a nucleic acid sequence having at least 90% identity to that of SEQ ID NO: 2. In some embodiments, the guide RNA that targets HPRT comprises a nucleic acid sequence having at least 95% identity to that of SEQ ID NO: 2. In some embodiments, the guide RNA targets the beta-globin locus. In some embodiments, the guide RNA that targets beta-globin comprises a nucleic acid sequence having at least 90% identity to that of SEQ ID NO: 3. In some embodiments, the guide RNA that targets beta-globin comprises a nucleic acid sequence having at least 95% identity to that of SEQ ID NO: 3.

In some embodiments, the guide RNA has at least 90% identity to any one of SEQ ID NOS: 39-54. In some embodiments, the guide RNA has at least 95% identity to any one of SEQ ID NOS: 39-54. In some embodiments, the guide RNA comprises any one of SEQ ID NOS: 39-54. In some embodiments, the guide RNA has at least 90% identity to any one of SEQ ID NOS: 1 and 4-23. In some embodiments, the guide RNA has at least 95% identity to any one of SEQ ID NOS: 1 and 4-23. In some embodiments, the guide RNA comprises any one of SEQ ID NOS: 1 and 4-23.

In some embodiments, the polymer shell is free from monomers or crosslinkers including a heterocyclic group. In some embodiments, the polymer shell is free from monomers or crosslinkers including an imidazole group. In some embodiments, the polymer shell is free of imidazolyl acryloyl monomers.

In a seventh aspect of the present disclosure is a method of treating a genetic condition in a human subject in need of treatment thereof comprising administering to the human subject a therapeutically effective amount of modified host cells, wherein the host cells are modified by: contacting host cells with either (i) a polymer nanocapsule comprising a polymer shell and a ribonucleoprotein complex, wherein the polymer shell comprises at least one positively charged monomer, at least one neutral monomer, and a crosslinker; or (ii) a polymer nanocapsule conjugate, wherein the conjugate comprises (a) a polymer shell and a ribonucleoprotein complex, wherein the polymer shell comprises at least one positively charged monomer, at least one neutral monomer, and a crosslinker; and (b) one or more targeting moieties and/or one or more stabilizing moieties. In some embodiments, the polymer shell comprises at least two positively charged monomers. In some embodiments, the host cells are hematopoietic stem cells. In some embodiments, the hematopoietic stem cells are allogenic hematopoietic stem cells. In some embodiments, the hematopoietic stem cells are autologous hematopoietic stem cells. In some embodiments, the hematopoietic stem cells are sibling matched hematopoietic stem cells.

In some embodiments, ribonucleoprotein complex comprises an endonuclease and a guide RNA. In some embodiments, the endonuclease is a Cas protein. In some embodiments, the Cas protein is selected from the group consisting of Cas9, Cas12a, and Cas12b. In some embodiments, the guide RNA targets the HPRT locus. In some embodiments, the guide RNA that targets HPRT comprises a nucleic acid sequence having at least 90% identity to that of SEQ ID NO: 2. In some embodiments, the guide RNA that targets HPRT comprises a nucleic acid sequence having at least 95% identity to that of SEQ ID NO: 2. In some embodiments, the guide RNA targets the beta-globin locus. In some embodiments, the guide RNA that targets beta-globin comprises a nucleic acid sequence having at least 90% identity to that of SEQ ID NO: 3. In some embodiments, the guide RNA that targets beta-globin comprises a nucleic acid sequence having at least 95% identity to that of SEQ ID NO: 3.

In some embodiments, the guide RNA has at least 90% identity to any one of SEQ ID NOS: 39-54. In some embodiments, the guide RNA has at least 95% identity to any one of SEQ ID NOS: 39-54. In some embodiments, the guide RNA comprises any one of SEQ ID NOS: 39-54. In some embodiments, the guide RNA has at least 90% identity to any one of SEQ ID NOS: 1 and 4-23. In some embodiments, the guide RNA has at least 95% identity to any one of SEQ ID NOS: 1 and 4-23. In some embodiments, the guide RNA comprises any one of SEQ ID NOS: 1 and 4-23.

In some embodiments, the polymer shell is free from monomers or crosslinkers including a heterocyclic group. In some embodiments, the polymer shell is free from monomers or crosslinkers including an imidazole group. In some embodiments, the polymer shell is free of imidazolyl acryloyl monomers.

In an eighth aspect of the present disclosure is a polymer nanocapsule comprising a polymer shell and a ribonucleoprotein complex, wherein the polymer shell comprises:

(i) at least one of:
N-(3-((4-((3-aminopropyl)amino)butyl)amino)propyl)acrylamide, and
2-(dimethylamino)ethyl acrylate;

(ii) acrylamide or a derivative thereof; and (iii) a crosslinker.

In some embodiments, the polymer shell comprises both N-(3-((4-((3-aminopropyl)amino)butyl)amino)propyl)acrylamide and 2-(dimethylamino)ethyl acrylate. In some embodiments, the crosslinker is an acrylate. In some embodiments, the acrylate is 2-(dimethylamino)ethyl acrylate. In some embodiments, the polymer nanocapsule has a diameter ranging from between about 50 nm to about 250 nm. In some embodiments, the polymer nanocapsule has a diameter ranging from between about 100 nm to about 200 nm. In some embodiments, the polymer shell is free from monomers or crosslinkers including a heterocyclic group. In some embodiments, the polymer shell is free from monomers or crosslinkers including an imidazole group. In some embodiments, the polymer shell is free of imidazolyl acryloyl monomers.

In some embodiments, ribonucleoprotein complex comprises an endonuclease and a guide RNA. In some embodiments, the endonuclease is a Cas protein. In some embodiments, the Cas protein is selected from the group consisting of Cas9, Cas12a, and Cas12b.

In some embodiments, the ribonucleoprotein complex includes a guide RNA that targets HPRT, a gamma-globin promoter, and/or beta-globin. In some embodiments, the guide RNA targets the HPRT locus. In some embodiments, the guide RNA that targets HPRT comprises a nucleic acid sequence having at least 90% identity to that of SEQ ID NO: 2. In some embodiments, the guide RNA that targets HPRT comprises a nucleic acid sequence having at least 95% identity to that of SEQ ID NO: 2.

In some embodiments, the guide RNA targets the beta-globin locus. In some embodiments, the guide RNA that targets beta-globin comprises a nucleic acid sequence having at least 90% identity to that of SEQ ID NO: 3. In some embodiments, the guide RNA that targets beta-globin comprises a nucleic acid sequence having at least 95% identity to that of SEQ ID NO: 3.

In some embodiments, the guide RNA has at least 90% identity to any one of SEQ ID NOS: 39-54. In some embodiments, the guide RNA has at least 95% identity to any one of SEQ ID NOS: 39-54. In some embodiments, the guide RNA comprises any one of SEQ ID NOS: 39-54. In some embodiments, the guide RNA has at least 90% identity to any one of SEQ ID NOS: 1 and 4-23. In some embodiments, the guide RNA has at least 95% identity to any one of SEQ ID NOS: 1 and 4-23. In some embodiments, the guide RNA comprises any one of SEQ ID NOS: 1 and 4-23.

In some embodiments, the polymer nanocapsule further comprises at least one targeting moiety. In some embodiments, the polymer nanocapsule comprises between two and six targeting moieties. In some embodiments, the at least one targeting moiety is coupled to the polymer nanocapsule through a spacer including a disulfide bond. In some embodiments, the at least one targeting moiety is an antibody.

In some embodiments, the at least one targeting moiety facilitates the delivery of the polymer nanocapsule to a specific cell type, wherein the cell type is selected from the group comprising immune cells, blood cells, cardiac cells, lung cells, optic cells, liver cells, kidney cells, brain cells, cells of the central nervous system, cells of the peripheral nervous system, cancer cells, cells infected with viruses, stem cells, skin cells, intestinal cells, and/or auditory cells. In some embodiments, the cancer cells are cells selected from the group comprising lymphoma cells, solid tumor cells, leukemia cells, bladder cancer cells, breast cancer cells, colon cancer cells, rectal cancer cells, endometrial cancer cells, kidney cancer cells, lung cancer cells, melanoma cells, pancreatic cancer cells, prostate cancer cells, and thyroid cancer cells.

In some embodiments, the polymer nanocapsule further comprises at least one stabilizing moiety. In some embodiments, the polymer nanocapsule comprises between two and six stabilizing moieties. In some embodiments, the at least one stabilizing moiety includes at least one repeat group selected from the group consisting of a polyethylene glycol repeat group and a polypropylene glycol repeat group. In some embodiments, the at least one stabilizing moiety includes at least two polyethylene glycol repeat groups. In some embodiments, the polymer nanocapsule comprises at least one targeting moiety and at least one stabilizing moiety.

In a ninth aspect of the present disclosure is a polymer nanocapsule conjugate comprising a polymer shell and a ribonucleoprotein complex, wherein the polymer nanocapsule includes at least one targeting moiety adapted to facilitate delivery of the ribonucleoprotein complex to a hematopoietic stem cell. In some embodiments, the targeting moiety is coupled to the polymer nanocapsule conjugate through a disulfide bond. In some embodiments, the at least one targeting moiety comprises an antibody. In some embodiments, the polymer nanocapsule conjugate further includes at least one stabilizing moiety having a hydrophilic group. In some embodiments, the hydrophilic group of the at least one stabilizing moiety comprises a polyethylene glycol group. In some embodiments, the hydrophilic group of the at least one stabilizing moiety is a polypropylene glycol repeat group.

In some embodiments, the polymer shell comprises at least one positively charged monomer. In some embodiments, the polymer shell comprises at least two positively charged monomers. In some embodiments, the positively charged monomers are selected from the group consisting of:
N-(3-((4-((3-aminopropyl)amino)butyl)amino) propyl)acrylamide,
N-(3-((4-((3-aminopropyl)amino)butyl)amino)propyl)methacrylamide,
N-(3-((4-aminobutyl)amino)propyl)acrylamide,
N-(3-((4-aminobutyl)amino)propyl)methacrylamide,
N-(2-((2-aminoethyl)(methyl)amino)ethyl)acrylamide,
N-(2-((2-aminoethyl)(methyl)amino)ethyl)methacrylamide,
N-(piperazin-1-ylmethyl)acrylamide,
N-(piperazin-1-ylmethyl)methacrylamide,
N-(2-(bis(2-aminoethyl)amino)ethyl)acrylamide,
N-(2-(bis(2-aminoethyl)amino)ethyl)methacrylamide,
N-(3-Aminopropyl)methacrylamidehydrochloride,
2-(dimethylamino)ethyl acrylate,
Dimethylamino ethyl methacrylate,
(3-Acrylamidopropyl) trimethylammonium hydrochloride,
2-aminoethyl methacrylate,
3-(Dimethylamino)propyl acrylate,
N-[3-(Dimethylamino)propyl]methacrylamide, and any combination thereof.

In some embodiments, polymer shell further comprises a neutral monomer and a crosslinker. In some embodiments, the polymer shell is free from monomers or crosslinkers including a heterocyclic group. In some embodiments, the polymer shell is free from monomers or crosslinkers including an imidazole group. In some embodiments, the polymer shell is free of imidazolyl acryloyl monomers.

In some embodiments, ribonucleoprotein complex comprises an endonuclease and a guide RNA. In some embodiments, the endonuclease is a Cas protein. In some embodiments, the Cas protein is selected from the group consisting of Cas9, Cas12a, and Cas12b.

In some embodiments, the ribonucleoprotein complex includes a guide RNA that targets HPRT, a gamma-globin promoter, and/or beta-globin. In some embodiments, the guide RNA targets the HPRT locus. In some embodiments, the guide RNA that targets HPRT comprises a nucleic acid sequence having at least 90% identity to that of SEQ ID NO: 2. In some embodiments, the guide RNA that targets HPRT comprises a nucleic acid sequence having at least 95% identity to that of SEQ ID NO: 2. In some embodiments, the guide RNA targets the beta-globin locus. In some embodiments, the guide RNA that targets beta-globin comprises a nucleic acid sequence having at least 90% identity to that of SEQ ID NO: 3. In some embodiments, the guide RNA that targets beta-globin comprises a nucleic acid sequence having at least 95% identity to that of SEQ ID NO: 3.

In some embodiments, the guide RNA has at least 90% identity to any one of SEQ ID NOS: 39-54. In some embodiments, the guide RNA has at least 95% identity to any one of SEQ ID NOS: 39-54. In some embodiments, the guide RNA comprises any one of SEQ ID NOS: 39-54. In some embodiments, the guide RNA has at least 90% identity to any one of SEQ ID NOS: 1 and 4-23. In some embodiments, the guide RNA has at least 95% identity to any one of SEQ ID NOS: 1 and 4-23. In some embodiments, the guide RNA comprises any one of SEQ ID NOS: 1 and 4-23.

In a tenth aspect of the present disclosure is a pharmaceutical composition comprising a polymer nanocapsule conjugate comprising a polymer shell and a ribonucleoprotein complex, wherein the polymer nanocapsule includes at least one targeting moiety adapted to facilitate delivery of the ribonucleoprotein complex to a hematopoietic stem cell. In some embodiments, the at least one targeting moiety is coupled to the polymer nanocapsule conjugate through a disulfide bond. In some embodiments, the polymer nanocapsule further includes at least one stabilizing moiety having a hydrophilic group, and (ii) a pharmaceutically acceptable carrier or excipient.

In some embodiments, the polymer shell comprises at least one positively charged monomer. In some embodiments, the polymer shell comprises at least two positively charged monomers. In some embodiments, the positively charged monomers are selected from the group consisting of:
N-(3-((4-((3-aminopropyl)amino)butyl)amino) propyl)acrylamide,
N-(3-((4-((3-aminopropyl)amino)butyl)amino)propyl)methacrylamide,
N-(3-((4-aminobutyl)amino)propyl)acrylamide,
N-(3-((4-aminobutyl)amino)propyl)methacrylamide,
N-(2-((2-aminoethyl)(methyl)amino)ethyl)acrylamide,
N-(2-((2-aminoethyl)(methyl)amino)ethyl)methacrylamide,
N-(piperazin-1-ylmethyl)acrylamide,
N-(piperazin-1-ylmethyl)methacrylamide,
N-(2-(bis(2-aminoethyl)amino)ethyl)acrylamide,
N-(2-(bis(2-aminoethyl)amino)ethyl)methacrylamide,
N-(3-Aminopropyl)methacrylamidehydrochloride,
2-(dimethylamino)ethyl acrylate,
Dimethylamino ethyl methacrylate,
(3-Acrylamidopropyl) trimethylammonium hydrochloride,
2-aminoethyl methacrylate,
3-(Dimethylamino)propyl acrylate,
N-[3-(Dimethylamino)propyl]methacrylamide, and any combination thereof.

In some embodiments, polymer shell further comprises a neutral monomer and a crosslinker. In some embodiments, the polymer shell is free from monomers or crosslinkers including a heterocyclic group. In some embodiments, the polymer shell is free from monomers or crosslinkers including an imidazole group. In some embodiments, the polymer shell is free of imidazolyl acryloyl monomers.

In some embodiments, ribonucleoprotein complex comprises an endonuclease and a guide RNA. In some embodiments, the endonuclease is a Cas protein. In some embodiments, the Cas protein is selected from the group consisting of Cas9, Cas12a, and Cas12b.

In some embodiments, the ribonucleoprotein complex includes a guide RNA that targets HPRT, a gamma-globin promoter, and/or beta-globin. In some embodiments, the guide RNA targets the HPRT locus. In some embodiments, the guide RNA that targets HPRT comprises a nucleic acid sequence having at least 90% identity to that of SEQ ID NO: 2. In some embodiments, the guide RNA that targets HPRT comprises a nucleic acid sequence having at least 95% identity to that of SEQ ID NO: 2. In some embodiments, the guide RNA targets the beta-globin locus. In some embodiments, the guide RNA that targets beta-globin comprises a nucleic acid sequence having at least 90% identity to that of SEQ ID NO: 3. In some embodiments, the guide RNA that targets beta-globin comprises a nucleic acid sequence having at least 95% identity to that of SEQ ID NO: 3.

In some embodiments, the guide RNA has at least 90% identity to any one of SEQ ID NOS: 39-54. In some embodiments, the guide RNA has at least 95% identity to any one of SEQ ID NOS: 39-54. In some embodiments, the guide RNA comprises any one of SEQ ID NOS: 39-54. In some embodiments, the guide RNA has at least 90% identity to any one of SEQ ID NOS: 1 and 4-23. In some embodiments, the guide RNA has at least 95% identity to any one of SEQ ID NOS: 1 and 4-23. In some embodiments, the guide RNA comprises any one of SEQ ID NOS: 1 and 4-23.

In an eleventh aspect of the present disclosure are modified hematopoictic stem cells prepared by contacting hematopoietic stem cells with a polymer nanocapsule conjugate comprising a polymer shell and a ribonucleoprotein complex, wherein the polymer nanocapsule includes at least one targeting moiety adapted to facilitate delivery of the ribonucleoprotein complex to a hematopoietic stem cell. In some embodiments, the wherein the targeting moiety is coupled to the polymer nanocapsule through a disulfide bond. In some embodiments, the polymer nanocapsule further includes at least one stabilizing moiety having a hydrophilic group. In some embodiments, the polymer shell comprises at least one positively charged monomer. In some embodiments, the polymer shell comprises at least two positively charged monomers. In some embodiments, the positively charged monomers are selected from the group consisting of:
N-(3-((4-((3-aminopropyl)amino)butyl)amino) propyl)acrylamide,
N-(3-((4-((3-aminopropyl)amino)butyl)amino)propyl)methacrylamide,
N-(3-((4-aminobutyl)amino)propyl)acrylamide,
N-(3-((4-aminobutyl)amino)propyl)methacrylamide,
N-(2-((2-aminoethyl)(methyl)amino)ethyl)acrylamide,
N-(2-((2-aminoethyl)(methyl)amino)ethyl)methacrylamide,
N-(piperazin-1-ylmethyl)acrylamide,
N-(piperazin-1-ylmethyl)methacrylamide,
N-(2-(bis(2-aminoethyl)amino)ethyl)acrylamide,
N-(2-(bis(2-aminoethyl)amino)ethyl)methacrylamide,
N-(3-Aminopropyl)methacrylamidehydrochloride,
2-(dimethylamino)ethyl acrylate,
Dimethylamino ethyl methacrylate,
(3-Acrylamidopropyl) trimethylammonium hydrochloride,
2-aminoethyl methacrylate,
3-(Dimethylamino)propyl acrylate,
N-[3-(Dimethylamino)propyl]methacrylamide, and any combination thereof.

In some embodiments, polymer shell further comprises a neutral monomer and a crosslinker. In some embodiments, the polymer shell is free from monomers or crosslinkers including a heterocyclic group. In some embodiments, the polymer shell is free from monomers or crosslinkers including an imidazole group. In some embodiments, the polymer shell is free of imidazolyl acryloyl monomers.

In some embodiments, ribonucleoprotein complex comprises an endonuclease and a guide RNA. In some embodiments, the endonuclease is a Cas protein. In some embodiments, the Cas protein is selected from the group consisting of Cas9, Cas12a, and Cas12b.

In some embodiments, the ribonucleoprotein complex includes a guide RNA that targets HPRT, a gamma-globin promoter, and/or beta-globin. In some embodiments, the guide RNA targets the HPRT locus. In some embodiments, the guide RNA that targets HPRT comprises a nucleic acid sequence having at least 90% identity to that of SEQ ID NO: 2. In some embodiments, the guide RNA that targets HPRT comprises a nucleic acid sequence having at least 95% identity to that of SEQ ID NO: 2. In some embodiments, the guide RNA targets the beta-globin locus. In some embodiments, the guide RNA that targets beta-globin comprises a nucleic acid sequence having at least 90% identity to that of SEQ ID NO: 3. In some embodiments, the guide RNA that targets beta-globin comprises a nucleic acid sequence having at least 95% identity to that of SEQ ID NO: 3.

In some embodiments, the guide RNA has at least 90% identity to any one of SEQ ID NOS: 39-54. In some embodiments, the guide RNA has at least 95% identity to any one of SEQ ID NOS: 39-54. In some embodiments, the guide RNA comprises any one of SEQ ID NOS: 39-54. In some embodiments, the guide RNA has at least 90% identity to any one of SEQ ID NOS: 1 and 4-23. In some embodiments, the guide RNA has at least 95% identity to any one of SEQ ID NOS: 1 and 4-23. In some embodiments, the guide RNA comprises any one of SEQ ID NOS: 1 and 4-23.

In a twelfth aspect of the present disclosure is a method of treating a human patient comprising administering a therapeutically effective amount of modified hematopoietic stem cells, wherein the hematopoietic stem cells are modified by: contacting hematopoietic stem cells with a polymer nanocapsule conjugate comprising a polymer shell and a ribonucleoprotein complex, wherein the polymer nanocapsule includes at least one targeting moiety adapted to facilitate delivery of the ribonucleoprotein complex to a hematopoietic stem cell. In some embodiments, the wherein the targeting moiety is coupled to the polymer nanocapsule through a disulfide bond. In some embodiments, the polymer nanocapsule further includes at least one stabilizing moiety having a hydrophilic group. In some embodiments, the polymer shell comprises at least one positively charged monomer. In some embodiments, the polymer shell comprises at least two positively charged monomers. In some embodiments, the positively charged monomers are selected from the group consisting of:
N-(3-((4-((3-aminopropyl)amino)butyl)amino) propyl)acrylamide,
N-(3-((4-((3-aminopropyl)amino)butyl)amino)propyl)methacrylamide,
N-(3-((4-aminobutyl)amino)propyl)acrylamide,
N-(3-((4-aminobutyl)amino)propyl)methacrylamide,
N-(2-((2-aminoethyl)(methyl)amino)ethyl)acrylamide,
N-(2-((2-aminoethyl)(methyl)amino)ethyl)methacrylamide,
N-(piperazin-1-ylmethyl)acrylamide,
N-(piperazin-1-ylmethyl)methacrylamide,
N-(2-(bis(2-aminoethyl)amino)ethyl)acrylamide,
N-(2-(bis(2-aminoethyl)amino)ethyl)methacrylamide,
N-(3-Aminopropyl)methacrylamidehydrochloride,
2-(dimethylamino)ethyl acrylate,
Dimethylamino ethyl methacrylate,
(3-Acrylamidopropyl) trimethylammonium hydrochloride,
2-aminoethyl methacrylate,
3-(Dimethylamino)propyl acrylate,
N-[3-(Dimethylamino)propyl]methacrylamide, and any combination thereof.

In some embodiments, polymer shell further comprises a neutral monomer and a crosslinker. In some embodiments, the polymer shell is free from monomers or crosslinkers including a heterocyclic group. In some embodiments, the polymer shell is free from monomers or crosslinkers including an imidazole group. In some embodiments, the polymer shell is free of imidazolyl acryloyl monomers.

In some embodiments, ribonucleoprotein complex comprises an endonuclease and a guide RNA. In some embodiments, the endonuclease is a Cas protein. In some embodiments, the Cas protein is selected from the group consisting of Cas9, Cas12a, and Cas12b.

In some embodiments, the ribonucleoprotein complex includes a guide RNA that targets HPRT, a gamma-globin promoter, and/or beta-globin. In some embodiments, the guide RNA targets the HPRT locus. In some embodiments, the guide RNA that targets HPRT comprises a nucleic acid sequence having at least 90% identity to that of SEQ ID NO: 2. In some embodiments, the guide RNA that targets HPRT comprises a nucleic acid sequence having at least 95% identity to that of SEQ ID NO: 2. In some embodiments, the guide RNA targets the beta-globin locus. In some embodiments, the guide RNA that targets beta-globin comprises a nucleic acid sequence having at least 90% identity to that of SEQ ID NO: 3. In some embodiments, the guide RNA that targets beta-globin comprises a nucleic acid sequence having at least 95% identity to that of SEQ ID NO: 3.

In some embodiments, the guide RNA has at least 90% identity to any one of SEQ ID NOS: 39-54. In some embodiments, the guide RNA has at least 95% identity to any one of SEQ ID NOS: 39-54. In some embodiments, the guide RNA comprises any one of SEQ ID NOS: 39-54. In some embodiments, the guide RNA has at least 90% identity to any one of SEQ ID NOS: 1 and 4-23. In some embodiments, the guide RNA has at least 95% identity to any one of SEQ ID NOS: 1 and 4-23. In some embodiments, the guide RNA comprises any one of SEQ ID NOS: 1 and 4-23.

In a thirteenth aspect of the present disclosure is a conjugate prepared according to a process comprising reacting a derivatized polymer nanocapsule with at least one derivatized targeting moiety, the derivatized polymer nanocapsule including at least one first reactive functional group capable of participating in a click chemistry reaction, the polymer nanocapsule having a polymer shell having at least one positively charged monomer, at least one neutral monomer, and a crosslinker; and wherein the at least one derivatized targeting moiety includes a second reactive functional group capable of participating in a click chemistry reaction with the first reactive functional group of the derivatized polymer nanocapsule. In some embodiments, the conjugate further comprises reacting the derivatized polymer nanocapsule with at least one derivatized stabilizing moiety, the at least one derivatized stabilizing moiety including a third reactive functional group capable of participating in a click chemistry reaction with the first reactive functional group. In some embodiments, the polymer shell comprises at least two positively charged monomers. In some embodiments, the polymer shell is free from monomers or crosslinkers including a heterocyclic group. In some embodiments, the polymer shell is free from monomers or crosslinkers including an imidazole group. In some embodiments, the polymer shell is free of imidazolyl acryloyl monomers.

In a fourteenth aspect of the present disclosure is a method of treating a genetic condition within a human patient comprising: generating a population of modified human hematopoietic stem cells by contacting an unmodified population of human hematopoietic stem cells with a polymer nanocapsule, the polymer nanocapsule including a polymer shell and a ribonucleoprotein complex, wherein the polymer shell comprises at least one positively charged monomer, at least one neutral monomer, and a crosslinker; and administering a therapeutically effective amount of the population of modified human hematopoietic stem cells to the human patient. In some embodiments, the polymer shell comprises at least two positively charged monomers. In some embodiments, the ribonucleoprotein complex comprises an endonuclease and a guide RNA. In some embodiments, the endonuclease is a Cas protein. In some embodiments, the Cas protein is selected from the group consisting of Cas9, Cas 12a, Cas12b. In some embodiments, the ribonucleoprotein complex comprises a Cas protein and a guide RNA. In some embodiments, the guide RNA targets one of the HPRT locus and the beta-globin locus. In some embodiments, the polymer shell is free from monomers or crosslinkers including a heterocyclic group. In some embodiments, the polymer shell is free from monomers or crosslinkers including an imidazole group. In some embodiments, the polymer shell is free of imidazolyl acryloyl monomers.

In a fifteenth aspect of the present disclosure is a conjugate comprising: (i) a polymer nanocapsule, wherein the polymer nanocapsule comprises a polymer shell and a ribonucleoprotein complex, wherein the polymer shell comprises at least one positively charged monomer, at least one neutral monomer, and a crosslinker; and (ii) at least one of (a) a targeting moiety, or (b) a stabilizing moiety, coupled to the polymer nanocapsule. In some embodiments, the polymer shell comprises at least two positively charged monomers. In some embodiments, the at least one of the targeting moiety or the stabilizing moiety is coupled to the polymer nanocapsule through at least one linker. In some embodiments, the at least one linker comprises a cleavable moiety. In some embodiments, the at least one linker comprises one or more PEG groups.

In some embodiments, the conjugate comprises between 1 and 16 targeting moieties. In some embodiments, the targeting moieties are selected from the group consisting of antibodies, peptides, lectins, transferrins, polysaccharides, nucleic acids, and aptamers. In some embodiments, the targeting moieties include human transferrin. In some embodiments, the targeting moieties are antibodies. In some embodiments, the antibodies are anti-cluster of differentiation marker antibodies. In some embodiments, the anti-cluster of differentiation marker antibodies are selected from the group consisting of anti-CD3 antibodies, anti-CD4 antibodies, anti-CD8 antibodies, anti-CD34 antibodies, anti-CD45 antibodies, anti-CD133 antibodies, and combinations thereof.

In some embodiments, the conjugate comprises between 1 and 16 stabilizing moieties. In some embodiments, the stabilizing moieties comprise one or more PEG groups. In some embodiments, the stabilizing moieties comprise one or more PPG groups. In some embodiments, the conjugate comprises one or more targeting moieties and one or more stabilizing moieties.

In some embodiments, the ribonucleoprotein complex comprises an endonuclease and a guide RNA. In some embodiments, wherein the endonuclease is a Cas protein. In some embodiments, the Cas protein is selected from the group consisting of Cas9 and Cas12a.

In some embodiments, the guide RNA targets the HPRT locus. In some embodiments, the guide RNA that targets HPRT comprises a nucleic acid sequence having at least 90% identity to that of SEQ ID NO: 2. In some embodiments, the guide RNA targets beta-globin. In some embodiments, the guide RNA that targets beta-globin comprises a nucleic acid sequence having at least 90% identity to that of SEQ ID NO: 3.

In some embodiments, the guide RNA has at least 90% identity to any one of SEQ ID NOS: 39-54. In some embodiments, the guide RNA has at least 95% identity to any one of SEQ ID NOS: 39-54. In some embodiments, the guide RNA comprises any one of SEQ ID NOS: 39-54. In some embodiments, the guide RNA has at least 90% identity to any one of SEQ ID NOS: 1 and 4-23. In some embodiments, the guide RNA has at least 95% identity to any one of SEQ ID NOS: 1 and 4-23. In some embodiments, the guide RNA comprises any one of SEQ ID NOS: 1 and 4-23.

In some embodiments, the at least one positively charged monomer is selected from the group consisting of:
N-(3-((4-((3-aminopropyl)amino)butyl)amino) propyl)acrylamide,
N-(3-((4-((3-aminopropyl)amino)butyl)amino)propyl)methacrylamide,
N-(3-((4-aminobutyl)amino)propyl)acrylamide,
N-(3-((4-aminobutyl)amino)propyl)methacrylamide,
N-(2-((2-aminoethyl)(methyl)amino)ethyl)acrylamide,
N-(2-((2-aminoethyl)(methyl)amino)ethyl)methacrylamide,
N-(piperazin-1-ylmethyl)acrylamide,
N-(piperazin-1-ylmethyl)methacrylamide,
N-(2-(bis(2-aminoethyl)amino)ethyl)acrylamide,
N-(2-(bis(2-aminoethyl)amino)ethyl)methacrylamide,
N-(3-Aminopropyl)methacrylamidehydrochloride,
2-(dimethylamino)ethyl acrylate,
Dimethylamino ethyl methacrylate,
(3-Acrylamidopropyl) trimethylammonium hydrochloride,
2-aminoethyl methacrylate,
3-(Dimethylamino)propyl acrylate,
N-[3-(Dimethylamino)propyl]methacrylamide, and
any combination thereof.

In some embodiments, the neutral monomer is selected from N-(1,3-dihydroxy-2-(hydroxymethyl)propan-2-yl)acrylamide, acrylamide, N-(hydroxymethyl)acrylamide, 2-hydroxyethyl acrylate, and 2-hydroxyethyl methacrylate. In some embodiments, the crosslinkers are selected from the group consisting of 1,3-glycerol dimethacrylate, N,N′-Methylenebisacrylamide, and glycerol 1,3-diglycerolate diacrylate. In some embodiments, the polymer shell is free from monomers or crosslinkers including a heterocyclic group. In some embodiments, the polymer shell is free from monomers or crosslinkers including an imidazole group. In some embodiments, the polymer shell is free of imidazolyl acryloyl monomers.

In a sixteenth aspect of the present disclosure is a (i) a polymer nanocapsule, wherein the polymer nanocapsule comprises a polymer shell and a payload, wherein the polymer shell comprises at least one positively charged monomer, at least one neutral monomer, and a crosslinker; and wherein the payload is selected from the group consisting of ribonucleoprotein complexes, siRNA molecules, shRNA molecules, expression vectors, polynucleotides having between 50 and 500 base pairs, peptides, enzymes, antibodies, and antibody fragments; and (ii) at least one of (a) a targeting moiety, or (b) a stabilizing moiety coupled to the polymer nanocapsule. In some embodiments, the polymer shell comprises at least two positively charged monomers. In some embodiments, the at least one positively charged monomer is selected from the group consisting of:
N-(3-((4-((3-aminopropyl)amino)butyl)amino) propyl)acrylamide,
N-(3-((4-((3-aminopropyl)amino)butyl)amino)propyl)methacrylamide,
N-(3-((4-aminobutyl)amino)propyl)acrylamide,
N-(3-((4-aminobutyl)amino)propyl)methacrylamide,
N-(2-((2-aminoethyl)(methyl)amino)ethyl)acrylamide,
N-(2-((2-aminoethyl)(methyl)amino)ethyl)methacrylamide,
N-(piperazin-1-ylmethyl)acrylamide,
N-(piperazin-1-ylmethyl)methacrylamide,
N-(2-(bis(2-aminoethyl)amino)ethyl)acrylamide,
N-(2-(bis(2-aminoethyl)amino)ethyl)methacrylamide,
N-(3-Aminopropyl)methacrylamidehydrochloride,
2-(dimethylamino)ethyl acrylate,
Dimethylamino ethyl methacrylate,
(3-Acrylamidopropyl) trimethylammonium hydrochloride,
2-aminoethyl methacrylate,
3-(Dimethylamino)propyl acrylate,
N-[3-(Dimethylamino)propyl]methacrylamide, and
any combination thereof.

In some embodiments, the neutral monomer is selected from N-(1,3-dihydroxy-2-(hydroxymethyl)propan-2-yl)acrylamide, acrylamide, N-(hydroxymethyl)acrylamide, 2-hydroxyethyl acrylate, and 2-hydroxyethyl methacrylate. In some embodiments, the crosslinkers are selected from the group consisting of 1,3-glycerol dimethacrylate, N,N′-Methylenebisacrylamide, and glycerol 1,3-diglycerolate diacrylate. In some embodiments, the conjugate comprises between 1 and 16 targeting moieties selected from the group consisting of antibodies, peptides, lectins, transferrins, polysaccharides, nucleic acids, and aptamers. In some embodiments, the conjugate comprises between 1 and 16 targeting moieties selected from the group consisting of antibodies and human transferrin. In some embodiments, the polymer shell is free from monomers or crosslinkers including a heterocyclic group. In some embodiments, the polymer shell is free from monomers or crosslinkers including an imidazole group. In some embodiments, the polymer shell is free of imidazolyl acryloyl monomers.

In a seventeenth aspect of the present disclosure is a polymer nanocapsule comprising a polymer shell and a ribonucleoprotein complex, wherein the polymer shell comprises at least one positively charged monomer, at least one neutral monomer, and a crosslinker, wherein the ribonucleoprotein complex comprises at least one gRNA, wherein the at least one gRNA has at least 90% sequence identity to any one of SEQ ID NOS: 1-22 and 39-54. In some embodiments, the gRNA has at least 95% sequence identity to any one of SEQ ID NOS: 1-22 and 39-54.

In a eighteenth aspect of the present disclosure is a polymer nanocapsule comprising a polymer shell and a ribonucleoprotein complex, wherein the polymer shell comprises at least one positively charged monomer, at least one neutral monomer, and a crosslinker, wherein the ribonucleoprotein complex comprises at least one gRNA, wherein the at least one gRNA comprises any one of SEQ ID NOS: 1-22 and 39-54.

In a nineteenth aspect of the present disclosure is a polymer nanocapsule comprising a polymer shell and a ribonucleoprotein complex, wherein the polymer shell comprises at least one positively charged monomer, at least one neutral monomer, and a crosslinker, wherein the ribonucleoprotein complex comprises at least one gRNA, wherein the at least one gRNA targets a nucleic acid sequence having at least 90% identity to any one of SEQ ID NOS: 24-37.

Applicant has developed nanocapsule technology with tunable chemistry and targeting capability that make it possible to specifically optimize the delivery of a wide variety of therapeutics. Applicant has been able to redirect nanocapsules to specifically target T lymphocytes or CD34+ hematopoietic stem cells by conjugating targeting/activating moieties to the nanocapsules to enable precise control of delivery. Applicant has shown that CRISPR/Cas9 ribonucleoprotein complexes formulated into these nanocapsules have less off-target effects and higher on-target efficiency. Applicant has also demonstrated that polymer nanocapsules can be formulated to include activating reagents that both enhance delivery and the gene editing process since it is believed that most T lymphocyte or CD34+ hematopoietic stem cells need to be activated to promote gene modification. Applicant further believes that it is possible to achieve high efficiency of gene modification and targeting capability simultaneously by incorporating CRISPR/Cas9 into the disclosed polymer nanocapsules.

### BRIEF DESCRIPTION OF THE FIGURES

For a general understanding of the features of the disclosure, reference is made to the drawings. In the drawings, like reference numerals have been used throughout to identify identical elements.

FIG. 1 illustrates the overall method of preparing targeted polymer nanocapsules bearing one or more stabilizing moieties.

FIG. 2 illustrates the synthesis of polymer nanocapsules including a ribonucleoprotein complex from different types of introduced monomers.

FIG. 3 illustrates the conjugation of targeting moieties to a polymer nanocapsule using copper-free click chemistry.

FIG. 4 provides GFP mRNA nanocapsules (unconjugated-unconjugated nanocapsules; TR1.1—nanocapsules conjugated with average 1.1 human transferrins; TR1.1PEG3.5—were nanocapsules conjugated with about 1.1 human transferrins and 3.5 PEGs) incubated with human serum protein solution (5 mg/mL) for 24 and 48 hours. Diameter of nanocapsules was determined by dynamic light scattering.

FIG. 5A illustrates GFP mRNA nanocapsules, each conjugated with a different average number of PEGs per particle, where each nanocapsule was tested in pre-stimulated PBMCs (nB-CD₃1.2—nanocapsules were conjugated with an average of about 1.2 anti-CD3 antibodies per particle; nB-CD₃1.2-PEG1.8—nanocapsules were conjugated with an average of about 1.2 CD3 antibodies per particle and an average of about 1.8 PEGs per particle; or nB-CD₃1.2-PEG3.4—nanocapsules were conjugated with an average of about 1.2 CD₃ antibodies per particle and an average of about 3.4 PEGs per particle). The nanocapsules were incubated with pre-stimulated PBMCs for 4 hours, then flow data was collected 3 days later. This represented a mock test with GFP mRNA for targeting CD3+ PBMC cells. The three samples each showed similar GFP expression levels, namely about 15% in a CD3+ population. This demonstrated that an average conjugation of about 1.8 and about 3.4 PEGs did not compromise the uptake of nanocapsules by the CD3+ subpopulation of cells. Both the nB-CD₃1.2-PEG3.4 and the nB-CD₃1.2-PEG1.8 nanocapsules showed a significant reduction of non-specific uptake by the CD3-subpopulation of PBMC cells (from 14% to about to about 4% and about 1%), demonstrating that an average conjugation of about 1.8 and about 3.4 PEGs per particle could reduce non-specific uptake by the CD3− population of PBMC cells.

FIG. 5B illustrates three GFP mRNA nanocapsules (nA-CD₃1.3, nB-CD₃1.2 and nC-CD₃0.9), each having a different surface zeta potential, tested in 293t cells. The positively charged nanocapsule nA-CD₃1.3 showed a higher GFP+ subpopulation (percentage) as compared with almost zero for the neutrally charged nB-CD₃1.2 nanocapsule, and the negatively charged nanocapsule nC-CD₃0.9. The nanocapsules were incubated with 293T cells for about 4 hours, and flow data was collected 3 days later. These results further demonstrate that gene editing was effective using nanocapsules of the present disclosure, such as those having a polymeric shell derived from a combination of positively charged and neutral monomers. Moreover, the results demonstrate that gene editing was effective using nanocapsules having a net positive charge.

FIG. 5C illustrates three GFP mRNA nanocapsules (nA-CD₃1.3, nB-CD31.2 and nC-CD₃0.9), each having a different surface zeta potential, tested in pre-stimulated PBMC cells. The positively charged nanocapsule nA-CD₃1.3 had a higher GFP+ subpopulation percentage (about 15%) as compared with almost zero for the neutrally charged nanocapsule nB-CD₃1.2 (about 13%). However, the negatively charged nanocapsule nC-CD₃0.9 showed a much lower GFP+ subpopulation percentage (about 6%). This demonstrated the impact of zeta potential on the uptake of nanocapsules in pre-stimulated PBMC cells. These results further demonstrate that gene editing was effective using nanocapsules of the present disclosure, such as those having a polymeric shell derived from a combination of positively charged and neutral monomers. Moreover, the results demonstrate that gene editing was effective using nanocapsules having a net positive charge.

FIG. 5D illustrates surface zeta potential of three GFP mRNA nanocapsules (nA-CD₃1.3, nB-CD₃1.2 and nC-CD₃0.9), which were measured to be about +9 mV, 0 mV and −5 mV, respectively, based on dynamic light scattering. Each GFP mRNA nanocapsule had a different formulation (i.e. the constituent parts, e.g. monomers, of each nanocapsule were different), but each nanocapsule formulation included a similar average number (1.3, 1.2 and 0.9) of conjugated CD3 targeting moieties per particle.

FIG. 5E illustrates GFP mRNA nanocapsules, each conjugated with a different average number of PEGs per particle, and which were tested in pre-stimulated PBMCs (nB-CD₃1.2—nanocapsules were conjugated with an average of about 1.2 anti-CD3 antibodies per particle; nB-CD₃1.2-PEG1.8—nanocapsules were conjugated with an average of about 1.2 CD3 antibodies per particle and an average of about 1.8 PEGs per particle; nB-CD₃1.2-PEG3.4—nanocapsules were conjugated with an average of about 1.2 CD3 antibodies per particle and an average of about 3.4 PEGs per particle). The nanocapsules were incubated with pre-stimulated PBMCs for about 4 hours, then flow data was collected 3 days later. This represented a mock test with GFP mRNA for confirmation of endocytosis through the CD3 receptor (see also FIGS. 5F and 5G). The three samples each showed similar GFP expression levels of about 15%, about 14% and about 12% in all PBMC populations.

FIG. 5F illustrates GFP mRNA nanocapsules conjugated with a different average number of PEGs per particle, which were tested in 293T cells as a model of CD3− cells (nB-CD₃1.2—nanocapsules were conjugated with an average 1.2 antiCD3 antibodies per particle; nB-CD₃1.2-PEG1.8—nanocapsules were conjugated with average of about 1.2 CD3 antibodies per particle and an average of about 1.8 PEGs per particle; nB-CD₃1.2-PEG3.4—nanocapsules were conjugated with an average of about 1.2 CD3 antibodies per particle and an average of about 3.4 PEGs per particle). The nanocapsules were incubated with 293T cells for about 4 hours, then flow data was collected 3 days later. The three samples each showed similar low GFP expression level of about 4%, about 2% and about 0% in CD3− 293T cells.

FIG. 5G illustrates GFP mRNA nanocapsules conjugated with a different average number of PEGs per particle, and which were tested in pre-stimulated PBMCs (nB-CD₃1.2-nanocapsules were conjugated with an average of about 1.2 antiCD3 antibodies per particle; nB-CD₃1.2-PEG1.8—nanocapsules were conjugated with an average of about 1.2 CD₃ antibodies per particle and an average of about 1.8 PEGs per particle; nB-CD₃1.2-PEG3.4—nanocapsules were conjugated with an average of about 1.2 CD₃ antibodies per particle and an average of about 3.4 PEG per particle). The nanocapsules were incubated with pre-stimulated PBMCs with 0.1 mg/mL of CD3 antibody in medium for about 4 hours, then flow data was collected 3 days later. This represented a mock test with GFP mRNA for confirmation of endocytosis through the CD3 receptor (see also FIGS. 5E and 5F). The GFP expression level of the three nanocapsules was reduced to about 5%, about 2% and about 0% in all PBMC populations as compared with FIG. 5F (without CD3 antibody in medium). This confirmed the three nanocapsules were taken up by PBMCs through a CD3-receptor-assisted endocytosis pathway.

FIG. 5H illustrates 5H GFP mRNA nanocapsules conjugated with a different average number of PEGs per particle, and which were tested by dynamic light scattering for solution stability over a time period of about 48 hours (unconjugated nanocapsules; nanocapsules conjugated with an average of about 1.1 human transferrin per particle; nanocapsules conjugated with an average of about 1.1 human transferrins per particle and an average of about 3.5 PEGs per particle). Unconjugated nanocapsules and nanocapsules conjugated with average of about 1.1 human transferrins per particle showed increased particle sizes and aggregation over a time period of about 48 hours. Nanocapsules conjugated with an average of about 1.1 human transferrins and about 3.5 PEGs per particle were stable in size and no significant formation of aggregates was observed.

FIGS. 6A (knockdown) and 6B (knock-out) illustrate the effect of positive selection with 6TG (ex vivo) on K562 cells. As shown in FIG. 6A, which did not include 6TG in the culture medium, K562 cells transduced with sh7-GFP vectors at MOI=1, 2 and 5 showed stable expression from day 3 to day 14. With about 300 nM of 6TG in the culture medium, GFP+ subpopulation of K562 cells transduced with sh7-GFP vectors at MOI=1, 2 and 5 showed a stable increase from about 19% to about 40%, about 40% to about 78%, and about 78% to about 94% from day 3 to day 14. As shown in FIG. 6B, without 6TG in the culture medium, K562 cells transfected with RNP targeting HPRT1 nanocapsules showed stable HPRT knockout INDEL of about 23% from day 3 to day 14. With about 300, about 600 and about 900 nM of 6TG present in the culture medium, the HPRT knockout population of K562 cells showed a stable increase to about 78%, about 95%, and about 97% at day 14. This confirmed positive selection from day 3 to day 14.

FIG. 7A (knockdown) and 7B (knock-out) illustrate the effect of positive selection with 6TG (ex vivo) at 0, 300, 600 and 900 nM on CEM cells transduced with sh7-GFP lentiviral vector or transfected with RNP targeting HPRT1 nanocapsules.

FIGS. 8A, 8B, and 8C illustrate the knock-out of HPRT and 6TG selection of PBMCs with CD3-conjugated CRISPR RNP targeting HPRT1 nanocapsules in accordance with some embodiments (see, for example, Example 11).

FIGS. 8D and 8E illustrate VCN and InDel data following selection of CD34+ transduced cells with sh734-rGbG/rGbG-sh734/sh734-GFP vectors or HPRT-KO CD34+ cells on 6-TG-treated methylcellulose plate.

FIGS. 9A and 9B illustrate the effect of negative selection with 300 nM of MTX on K562 cells transduced with sh7-GFP lentiviral vector or transfected with RNP targeting HPRT1 nanocapsules.

FIGS. 10A and 10B illustrate the effect of negative selection with 300 nM of MTX, 1 uM and 10 uM of MPA on CEM cells transduced with sh7-GFP lentiviral vector or transfected with RNP targeting HPRT1 nanocapsules.

FIG. 11A illustrates gene editing to knock-out CCR5 in PBMCs. 1×10⁵ PBMCs were incubated with CD3-conjugated Cas9-CCR5 RNP nanocapsule (150 ng Cas9 and 100 ng gRNA targeting the CCR5 locus) and C46 nanocapsule (100 ng or 200 ng short EF1a-driven-C46-expression DNA cassette) for 4 hours. Then, PHA/IL-2 was used to stimulate PBMC overnight. Five days later, flow results shown in FIG. 11A. The top left panel of FIG. 11A shows that unstained negative control have >99% of the CCR5 subpopulation; the top middle panel shows that CCR5 stained stimulated PBMC comprise about 41.2% of the CCR5-subpopulation; the top right panel shows PBMCs treated with CCR5-targeting RNP nanocapsules have 75.8% of the CCR5− subpopulation; the bottom left panel shows that PBMCs treated with CCR5-targeting RNP nanocapsules and C46 nanocapsules (100 ng) have 69.7% of the CCR5-subpopulation; the bottom middle panel shows PBMCs treated with CCR5-targeting RNP nanocapsules and C46 nanocapsule (200 ng) have 63.7% of CCR5-subpopulation; the bottom right panel shows PBMCs transduced with Cal-1 at MOI=5 have 92.9 of CCR5-subpopulation. T7E1 analysis was carried out (results of which are shown in FIG. 11B).

FIG. 11B illustrates T7E1 assay showing knock-out of CCR5. From left to right: CD3-conjugated CCR5-targeting RNP nanocapsule (150 ng Cas9 and 100 ng gRNA targeting the CCR5 locus); CD3-conjugated CCR5-targeting RNP nanocapsule (150 ng of Cas9 and 100 ng of gRNA targeting the CCR5 locus) and C46 nanocapsule (100 ng of short EF1a-driven-C46-expression DNA cassette); CD3-conjugated CCR5-targeting RNP nanocapsule (150 ng Cas9 and 100 ng gRNA targeting the CCR5 locus) and C46 nanocapsule (200 ng of short EF1a-driven-C46-expression DNA cassette); Control of unmodified cells; DNA ladder.

FIG. 12 provides data illustrating the delivery of a gCCR5-Cas9 RNP via a nanocapsule designed to target CD3+ PBMCs. 1×10⁵ of unstimulated PBMCs were incubated with antibody CD3-conjugated Cas9 RNP nanocapsules (1/2/4 pmol) for 4 hours. Then, the PBMCs were stimulated with PHA/IL-2 overnight and cultured with IL-2 for 4 days before staining. Overall, CCR5 expression of stimulated PBMC in CD3+ subpopulation was shown to decrease when the Cas9 RNP nanocapsule dosage decreased from 55.4% (control) to 34.6% (1 pmol of RNP nanocapsule targeting the CCR5 locus), 27.0% (2 pmol of RNP nanocapsule targeting the CCR5 locus) and 20.5% (5 pmol of RNP nanocapsules targeting the CCR5 locus).

FIGS. 13A and 13B illustrate the preparation of a nanocapsule including at least a Cas9 and a gRNA.

FIG. 14 illustrates T7E1 assay results for control, nanoRNP and nanohdr DNA for knockout 293T cells.

FIG. 15 provides flow cytometry data. Panels A and B show the CCR5 staining result from untreated Mocha (˜95% of CCR5+) cells and treated MOCHA. Panels C, D, and E show C46 staining results of untreated Mocha, CCR5− subpopulation of treated Mocha and C46-knock-in AW072 cell line.

FIG. 16 Panels A, B, and C showed unstained control, CCR5 staining result from CD34+ cells without 6TG treatment (44.9% of CCR5+) (FIG. 16, panel B) and with 6TG treatment (43.4% of CCR5+) (FIG. 16, panel C). FIG. 16 panel D showed CCR5+ stained results from nanocapsule-treated CD34+ cells without 6TG in culture media (23.8%) and with 6TG in culture media (10.2%).

FIG. 17 showed unstained control, CCR5 staining result from CD34+ cells without 6TG treatment (44.9% of CCR5+) with 6TG treatment (43.4% of CCR5+). FIG. 17 further showed CCR5+ stained results from nanocapsule-treated CD34+ cells without 6TG in culture media (23.8%) and with 6TG in culture media (10.2%). CCR5 staining data suggest 6-TG selection happens for sh734-KI mPB CD34+ cells on bulk culture.

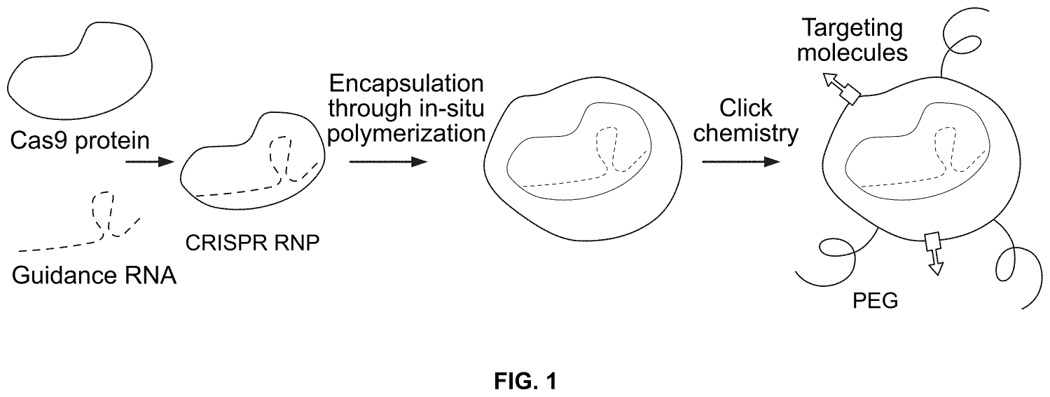

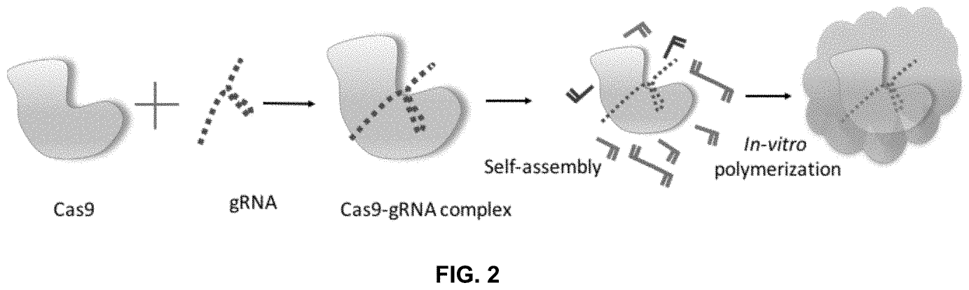

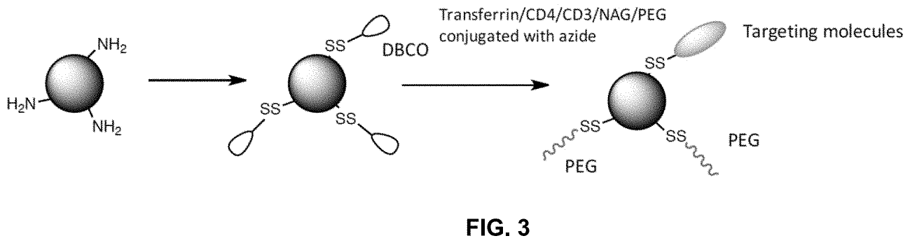

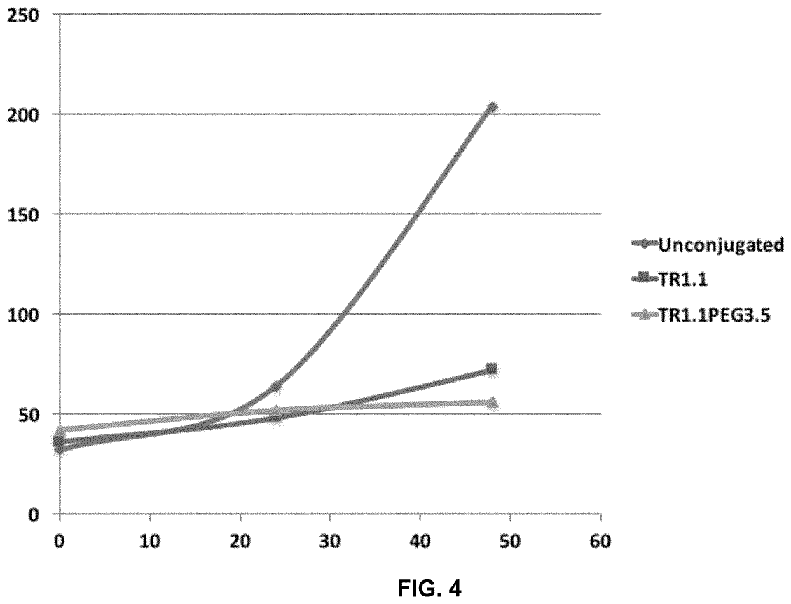

### SEQUENCE LISTING

The nucleic acid and amino acid sequences appended hereto are shown using standard letter abbreviations for nucleotide bases, and three letter code for amino acids, as defined in 37 C.F.R. 1.822. The sequence listing is submitted as an ASCII text file, named “Calimmune-042WO_ST25.txt” created on Dec. 21, 2019, 12 KB, which is incorporated by reference herein.

### DETAILED DESCRIPTION

It should also be understood that, unless clearly indicated to the contrary, in any methods claimed herein that include more than one step or act, the order of the steps or acts of the method is not necessarily limited to the order in which the steps or acts of the method are recited.

As used herein, the singular terms “a,” “an,” and “the” include plural referents unless context clearly indicates otherwise. Similarly, the word “or” is intended to include “and” unless the context clearly indicates otherwise. The term “includes” is defined inclusively, such that “includes A or B” means including A, B, or A and B.

As used herein, the term “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of” or “exactly one of.” “Consisting essentially of,” when used in the claims, shall have its ordinary meaning as used in the field of patent law.

As used herein, the terms “comprising,” “including,” “having,” and the like are used interchangeably and have the same meaning. Similarly, “comprises,” “includes,” “has,” and the like are used interchangeably and have the same meaning. Specifically, each of the terms is to be interpreted to be an open term meaning “at least the following,” and is also interpreted not to exclude additional features, limitations, aspects, etc. Thus, for example, “a device having components a, b, and c” means that the device includes at least components a, b and c. Similarly, the phrase: “a method involving steps a, b, and c” means that the method includes at least steps a, b, and c. Moreover, while the steps and processes may be outlined herein in a particular order, the skilled artisan will recognize that the ordering steps and processes may vary.

As used herein, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, the term “administering” refers to an administration that is oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

As used herein, the term “alkyl” refers to a straight or branched hydrocarbon chain that comprises a fully saturated (no double or triple bonds) hydrocarbon group. The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. The term “alkyl” further includes alkyl groups, wherein one or more heteroatoms, such as oxygen, nitrogen, sulfur or phosphorous, replaces one or more carbons of the hydrocarbon backbone. In certain embodiments, a straight chain or branched chain alkyl has 30 or fewer carbon atoms in its backbone (e.g., C₁-C₃₀ for straight chain, C₁-C₃₀ for branched chain). Moreover, the term “alkyl” includes both “unsubstituted alkyls” and “substituted alkyls”, the latter of which refers to alkyl moieties having substituents replacing a hydrogen on one or more carbons of the hydrocarbon backbone. Such substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used herein, the term “alkene” includes unsaturated aliphatic groups analogous in length and possible substitution to the alkyls described above, but that contain at least one double bond. For example, the term “alkene” includes straight-chain alkenyl groups (e.g., ethylenyl, propenyl, butenyl, pentenyl, hexenyl, heptenyl, octenyl, nonenyl, decenyl, etc.), branched-chain alkenyl groups, cycloalkenyl (alicyclic) groups (cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. The term alkenyl further includes alkenyl groups which include oxygen, nitrogen, sulfur or phosphorous atoms replacing one or more carbons of the hydrocarbon backbone. In certain embodiments, a straight chain or branched chain alkenyl group has 30 or fewer carbon atoms in its backbone (e.g., C₂-C₃₀ for straight chain, C₃-C₃₀ for branched chain). Moreover, the term alkenyl includes both “unsubstituted alkenyls” and “substituted alkenyls,” the latter of which refers to alkenyl moieties having substituents replacing a hydrogen on one or more carbons of the hydrocarbon backbone. Such substituents can include, for example, alkyl groups, alkenyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Other examples of alkenyl groups include, but are not limited to, ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl; 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl groups. Groups containing multiple double bonds may include but are not limited to buta-1,3-dienyl, penta-1,3-dienyl or penta-1,4-dienyl groups.

As used herein, the terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Cycloalkyls and heterocycloalkyl can be further substituted, e.g., with any of the substituents described herein.

By way of example only, an alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). As noted further herein, the alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from methyl, ethyl, propyl, iso-propyl, n-butyl, iso-butyl, sec-butyl, and t-butyl.

As used herein, the term “antibody,” refers to immunoglobulins or immunoglobulin-like molecules, including by way of example and without limitation, IgA, IgD, IgE, IgG and IgM, combinations thereof, and similar molecules produced during an immune response in any vertebrate, (e.g., in mammals such as humans, goats, rabbits and mice) and antibody fragments (such as F(ab′)2 fragments, Fab′ fragments, Fab′-SH fragments and Fab fragments as are known in the art, recombinant antibody fragments (such as sFv fragments, dsFv fragments, bispecific sFv fragments, bispecific dsFv fragments, F(ab)′2 fragments, single chain Fv proteins (“scFv”), disulfide stabilized Fv proteins (“dsFv”), diabodies, and triabodies (as are known in the art), and camelid antibodies) that specifically bind to a molecule of interest (or a group of highly similar molecules of interest) to the substantial exclusion of binding to other molecules. Antibody further refers to a polypeptide ligand comprising at least a light chain or heavy chain immunoglobulin variable region which specifically recognizes and binds an epitope of an antigen. Antibodies may be composed of a heavy and a light chain, each of which has a variable region, termed the variable heavy (VH) region and the variable light (VL) region. Together, the VH region and the VL region are responsible for binding the antigen recognized by the antibody. The term antibody also includes intact immunoglobulins and the variants and portions of them well known in the art.

As used herein, the terms “B-cell lymphoma/leukemia 11A” or “BCL11A” is encodes a C2H2 type zinc-finger protein by its similarity to the mouse Bcl11a/Evi9 protein. The corresponding mouse gene is a common site of retroviral integration in myeloid leukemia, and may function as a leukemia disease gene, in part, through its interaction with BCL6. During hematopoietic cell differentiation, this gene is down-regulated. It is possibly involved in lymphoma pathogenesis since translocations associated with B-cell malignancies also deregulates its expression. Multiple transcript variants encoding several different isoforms have been found for this gene.

As used herein, the term “C9orf72” refers to a gene which provides instructions for making a protein that is found in various tissues. The protein is abundant in nerve cells (neurons) in the outer layers of the brain (cerebral cortex) and in specialized neurons in the brain and spinal cord that control movement (motor neurons). The C9orf72 protein is thought to be located at the tip of the neuron in a region called the presynaptic terminal. This area is important for sending and receiving signals between neurons.

As used herein, “Cₐ to C_{b}” or “Cₐ-C_{b}” in which “a” and “b” are integers refer to the number of carbon atoms in an alkyl, alkenyl or alkynyl group, or the number of carbon atoms in the ring of a cycloalkyl, cycloalkenyl, cycloalkynyl or aryl group, or the total number of carbon atoms and heteroatoms in a heteroalkyl, heterocyclyl, heteroaryl or heteroalicyclyl group. That is, the alkyl, alkenyl, alkynyl, ring of the cycloalkyl, ring of the cycloalkenyl, ring of the cycloalkynyl, ring of the aryl, ring of the heteroaryl or ring of the heteroalicyclyl can contain from “a” to “b”, inclusive, carbon atoms. Thus, for example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)₃— and (CH₃)₃C—. If no “a” and “b” are designated with regard to an alkyl, alkenyl, alkynyl, cycloalkyl cycloalkenyl, cycloalkynyl, aryl, heteroaryl or heteroalicyclyl group, the broadest range described in these definitions is to be assumed.

As used herein, the term “Cas protein” refers to an RNA-guided nuclease comprising a Cas protein, or a fragment thereof. A Cas protein may also be referred to as a CRISPR (clustered regularly interspaced short palindromic repeat)-associated nuclease. CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). CRISPR-Cas systems may be further characterized as Class 1 or Class 2 systems. Class 1 systems are characterized by multi-subunit effector; that is, comprising multiple Cas proteins. Class 1 systems may be further characterized as Types I, III and IV. Class 2 systems are characterized by a single effector protein having multiple domains. Class 1 systems may be further characterized as Types II, V and VI. For example, Class 2 type II systems include Cas9 while Class 2 type V systems include Cpf1 (Cas 12a). Further examples of Cas proteins include, but are not limited to, Cas9 proteins, Cas9-like proteins encoded by Cas9 orthologs, Cas9-like synthetic proteins, Cpf1 proteins, proteins encoded by Cpf1 orthologs, Cpf1-like synthetic proteins, C2c1 proteins, C2c2 proteins, C2c3 proteins, and variants and modifications thereof. Further examples of Cas proteins include, but are not limited to, MAD7, MAD2, Cpf1, C2c1, C2c3, Cas12a, Cas12b, Cas12c, Cas12d, Cas12e, Cas13a, Cas13b, and Cas13c. Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, and Cas13c.

In some embodiments, the Cas protein is a Class 2 CRISPR-associated protein. As noted above, “Class 2 type CRISPR-Cas systems” as defined herein refer to CRISPR-Cas systems functioning with a single protein as effector complex (such as Cas9). As defined herein, “class 2 type II CRISPR-Cas system” refers to CRISPR-Cas systems comprising the cas9 gene among its Cas genes. A “class 2 type II-A CRISPR-Cas system” refers to CRISPR-Cas systems comprising Cas9 and Csn2 genes. A “class 2 type II-B CRISPR-Cas system” refers to CRISPR-Cas systems comprising the Cas9 and Cas4 genes. A “class 2 type II-C CRISPR-Cas system” refers to CRISPR-Cas systems comprising the Cas9 gene but neither the Csn2 nor the Cas4 gene. A “class 2 type V CRISPR-Cas system” refers to CRISPR-Cas systems comprising the Cas12 gene (Cas12a, Cas12b or Cas12c gene) in its Cas genes. A “class 2 type VI CRISPR-Cas system” refers to CRISPR-Cas systems comprising the cas13 gene (cas13a, 13b or 13c gene) in its Cas genes. Each wild-type CRISPR-Cas protein interacts with one or more cognate polynucleotide (most typically RNA) to form a nucleoprotein complex (most typically a ribonucleoprotein complex). Additional Cas proteins are described by Haft et. al., “A Guild of 45 CRISPR-Associated (Cas) Protein Families and Multiple CRISPR/Cas Subtypes Exist in Prokaryotic Genomes, PLoS Comput. Biol., 2005, November; 1(6): e60. In some embodiments, the Cas protein is a modified Cas protein, e.g. a modified variant of any of the Cas proteins identified herein.

As used herein, the terms “Cas9” or “Cas9 protein” refer to an enzyme (wild-type or recombinant) that can exhibit endonuclease activity (e.g. cleaving the phosphodiester bond within a polynucleotide) guided by a CRISPR RNA (crRNA) bearing complementary sequence to a target polynucleotide. Cas9 polypeptides are known in the art and include Cas9 polypeptides from any of a variety of biological sources, including, e.g., prokaryotic sources such as bacteria and archaea. Bacterial Cas9 includes, Actinobacteria (e.g., Actinomyces naeslundii) Cas9, Aquificae Cas9, Bacteroidetes Cas 9, Chlamydiae Cas9, Chloroflexi Cas9, Cyanobacteria Cas9, Elusimicrobia Cas9, Fibrobacteres Cas9, Firmicutes Cas9 (e.g., Streptococcus pyogenes Cas9, Streptococcus thermophilus Cas9, Listeria innocua Cas9, Streptococcus agalactiae Cas9, Streptococcus mutans Cas9, and Enterococcus faecium Cas9), Fusobacteria Cas9, Proteobacteria (e.g., Neisseria meningitides, Campylobacter jejuni and lari) Cas9, Spirochaetes (e.g., Treponema denticola) Cas9, and the like. Archaea Cas 9 includes Euryarchaeota Cas9 (e.g., Methanococcus maripaludis Cas9) and the like. A variety of Cas9 and related polypeptides are known, and are reviewed in, e.g., Makarova et al. (2011) Nature Reviews Microbiology 9:467-477, Makarova et al. (2011) Biology Direct 6:38, Haft et al. (2005) PLOS Computational Biology I:e60 and Chylinski et al. (2013) RNA Biology 10:726-737; K. Makarova et al., An updated evolutionary classification of CRISPR-Cas systems. (2015) Nat. Rev. Microbio. 13:722-736; and B. Zetsche et al. Cpf1 is a single RNA-guided endonuclease of a class 2 CRISPR-Cas system. (2015) Cell. 163(3):759-771. Other Cas9 polypeptides can be Francisella tularensis subsp. novicida Cas9, Pasteurella multocida Cas9, Mycoplasma gallisepticum str. F Cas9, Nitratifractor salsuginis str DSM 16511 Cas9, Parvibaculum lavamentivorans Cas9, Roseburia intestinalis Cas9, Neisseria cinera Cas9, Gluconacetobacter diazotrophicus Cas9, Azospirillum B510 Cas9, Spaerochaeta globus str. Buddy cas9, Flavobacterium columnare Cas9, Fluviicola taffensis Cas9, Bacteroides coprophilus Cas9, mycoplasma mobile Cas9, Lactobacillus farciminis Cas9, Streptococcus pasteurianus Cas9, Lactobacillus johnsonii Cas9, Staphylococcus pseudintermedius Cas9, filifactor alocis Cas9, Treponema denticola Cas9, Legionella pneumophila str. Paris Cas9, Sutterella wadsworthensis Cas9, and Corynebacter diptheriae Cas9. The term “Cas9” includes a Cas9 polypeptide of any Cas9 family, including any isoform of Cas9. Amino acid sequences of various Cas9 homologs, orthologs, and variants beyond those specifically stated or provided herein are known in the art and are publicly available, within the purview of those skill in the art, and thus within the spirit and scope of this disclosure.

As used herein, the terms “Cas12” or “Cas12 protein” refer to any Cas12 protein including, but not limited to, Cas12 protein such as Cas12a, Cas12b, Cas12c, Cas12d, Cas12e. In some embodiments, a Cas12 protein has an amino acid sequence which is at least 85% (or at least 90%, or at least 95%, or at least 96%, or at least 97%, or at least 98%, or at least 99%) identical to the amino acid sequence of a functional Cas12 protein, particularly the Cas12a/Cpf1 protein from Acidaminococcus sp. strain BV3L6 (Uniprot Entry: U2UMQ6; Uniprot Entry Name: CS12A_ACISB) or the Cas12a/Cpf1 protein from Francisella tularensis (Uniprot Entry: AOQ7Q2; Uniprot Entry Name: CS12A_FRATN). In some embodiments, the Cas12 protein may be a Cas12 polypeptide substantially identical to the protein found in nature, or a Cas12 polypeptide having at least 85% sequence identity (or at least 90% sequence identity, or at least 95% sequence identity, or at least 96% sequence identity, or at least 97% sequence identity, or at least 98% sequence identity, or at least 99% sequence identity) to the Cas12 protein found in nature and having substantially the same biological activity. Examples of Cas12a proteins include, but are not limited to, FnCas12a, AsCas12a, LbCas12a, Lb5Cas12a, HkCas12a, OsCas12a, TsCas12a, BbCas12a, BoCas12a or Lb4Cas12a; the Cas12a is preferably LbCas12a. Examples of Cas12b proteins include, but are not limited to, AacCas12b, Aac2Cas12b, AkCas12b, AmCas12b, AhCas12b, AcCas12b.

As used herein, the term “CCR5” refers to C-C chemokine receptor type 5, which is a protein on the surface of white blood cells that is involved in the immune system as it acts as a receptor for chemokines. This is the process by which T cells are attracted to specific tissue and organ targets. Many forms of HIV, the virus that causes AIDS, initially use CCR5 to enter and infect host cells. Certain individuals carry a mutation known as CCR5-A32 in the CCR5 gene, protecting them against these strains of HIV.

As used herein, the term “conjugate” refers to two or more molecules or moieties (including macromolecules or supra-molecular molecules) that are covalently linked, bonded, or otherwise coupled together into a larger construct.

As used herein, the term “crosslinker” refers to a bond or moiety which provides an link (e.g. an intramolecular link or intermolecular link) between two or more molecular chains, domains, or other moieties. In some embodiments, a crosslinker is a molecule which forms links between molecular chains to form a connected molecule.

As used herein, the term “endocytosis” as used herein, refers to a form of active transport in which a cell transports molecules (such as proteins) into the cell by engulfing them in an energy-using process. Endocytosis includes pinocytosis and phagocytosis. Pinocytosis is a mode of endocytosis in which small particles are brought into the cell, forming an invagination, and then suspended within small vesicles. These pinocytotic vesicles subsequently fuse with lysosomes to hydrolyze (break down) the particles. Phagocytosis is the process by which a cell engulfs a solid particle to form an internal compartment known as a phagosome.

As used herein, the phrase “effective amount” refers to the amount of a composition or formulation described herein that will elicit the diagnostic, biological or medical response of a tissue, system, animal, or human that is being sought by the researcher, veterinarian, medical doctor or other clinician.

As used herein, the term “gene” refers broadly to any segment of DNA associated with a biological function. A gene encompasses sequences including but not limited to a coding sequence, a promoter region, a cis-regulatory sequence, a non-expressed DNA segment is a specific recognition sequence for regulatory proteins, a non-expressed DNA segment that contributes to gene expression, a DNA segment designed to have desired parameters, or combinations thereof.

As used herein, the terms “guide polynucleotide,” “guide RNA,” or “gRNA” may refer to any polynucleotide sequence having sufficient complementarity with a target polynucleotide sequence to hybridize with the target sequence and direct sequence-specific binding of a CRISPR complex to the target sequence. The degree of complementarity between a guide polynucleotide and its corresponding target sequence, when optimally aligned using a suitable alignment algorithm, can be about or more than about 50%, 60%, 75%, 80%, 85%, 90%, 95%, 97.5%, 99%, or more. In some embodiments, the degree of complementarity between a guide polynucleotide and its corresponding target sequence, when optimally aligned using a suitable alignment algorithm can be less than about 50%, such as 40%, 30%, 20% or less. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (lllumina, San Diego, Calif.), SOAP (available at soap.genomics.org.cn), and Maq (available at maq.sourceforge.net). A guide polynucleotide (also referred to herein as a guide sequence and includes single guide sequences (sgRNA)) can be about or more than about 5, 10, 11, 12, 13, 14, 15, 16, 17, 18, 19, 20, 21, 22, 23, 24, 25, 26, 27, 28, 29, 30, 35, 40, 45, 50, 75, 90, 100, 110, 112, 115, 120, 130, 140, or more nucleotides in length.

The guide polynucleotide can include a nucleotide sequence that is complementary to a target DNA sequence. This portion of the guide sequence can be referred to as the complementary region of the guide RNA. In some contexts, the two are distinguished from one another by calling one the complementary region or target region and the rest of the polynucleotide the guide sequence or tracrRNA. The guide sequence can also include one or more miRNA target sequences coupled to the 3′ end of the guide sequence. The guide sequence can include one or more MS2 RNA aptamers incorporated within the portion of the guide strand that is not the complementary portion. As used herein the term guide sequence can include any specially modified guide sequences, including but not limited to those configured for use in synergistic activation mediator (SAM) implemented CRISPR (Nature 517, 583-588 (29 Jan. 2015)). A guide polynucleotide can be less than about 150, about 125, about 75, about 50, about 45, about 40, about 35, about 30, about 25, about 20, about 15, about 12, or fewer nucleotides in length. The ability of a guide polynucleotide to direct sequence-specific binding of a CRISPR complex to a target sequence may be assessed by any suitable assay. For example, the components of a CRISPR system sufficient to form a CRISPR complex, including the guide polynucleotide to be tested, may be provided to a host cell having the corresponding target sequence, such as by transfection with vectors encoding the components of the CRISPR sequence, followed by an assessment of preferential cleavage within the target sequence or by any of the delivery systems provided elsewhere herein. Similarly, cleavage of a target polynucleotide sequence may be evaluated in a test tube by providing the target sequence, components of a CRISPR complex, including the guide polynucleotide to be tested and a control guide polynucleotide different from the test guide polynucleotide, and comparing binding or rate of cleavage at the target sequence between the test and control guide polynucleotide reactions. Other assays are possible, and will occur to those skilled in the art.

As used herein, the terms “hemoglobin subunit beta” or “HBB” refer to a gene that provides instructions for making a protein called beta-globin. Beta-globin is a component (subunit) of a larger protein called hemoglobin, which is located inside red blood cells. In adults, hemoglobin normally consists of four protein subunits: two subunits of beta-globin and two subunits of another protein called alpha-globin, which is produced from another gene called HBA. Each of these protein subunits is attached (bound) to an iron-containing molecule called heme; each heme contains an iron molecule in its center that can bind to one oxygen molecule. Hemoglobin within red blood cells binds to oxygen molecules in the lungs. These cells then travel through the bloodstream and deliver oxygen to tissues throughout the body.

As used herein, the term “heteroalkyl,” by itself or in combination with another term, means, unless otherwise stated, a stable straight or branched chain, or combinations thereof, consisting of at least one carbon atom and at least one heteroatom selected from the group consisting of O, N, P, Si, and S, and wherein the nitrogen, phosphorus, and sulfur atoms may optionally be oxidized, and the nitrogen heteroatom may optionally be quaternate. The heteroatom(s) O, N, P, S, and Si may be placed at any interior position of the heteroalkyl group or at the position at which the alkyl group is attached to the remainder of the molecule. A heteroalkyl is not cyclized. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—O—CH₃, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two heteroatoms may be consecutive, such as, for example, —CH₂—NH—OCH₃.

The term “heteroatom” as used herein, refers to an atom other tan carbon or hydrogen. Examples include, nitrogen, oxygen, sulfur, phosphorus, chlorine, bromine, and iodine.

As used herein, the terms “host cell” or “target cell” refer to cells that is to be modified using the methods of the present disclosure. Suitable mammalian host cells include, but are not limited to, human cells, murine cells, non-human primate cells (e.g. rhesus monkey cells), human progenitor cells or stem cells, 293 cells, HeLa cells, D17 cells, MDCK cells, BHK cells, and Cf2Th cells. In some embodiments, the host cells are hematopoietic cells, such as hematopoietic progenitor/stem cells (e.g. CD34-positive hematopoietic progenitor/stem cell), a monocyte, a macrophage, a peripheral blood mononuclear cell, a CD4+ T lymphocyte, a CD8+ T lymphocyte, or a dendritic cell. In some embodiments, the hematopoietic cells (e.g. CD4+ T lymphocytes, CD8+ T lymphocytes, and/or monocyte/macrophages) to be transfected with the polymer nanocapsules or polymer nanocapsule conjugates of the disclosure may be allogeneic, autologous, or from a matched sibling. The hematopoietic progenitor/stem cells are, in some embodiments, CD34-positive and can be isolated from the patient's bone marrow or peripheral blood. The isolated CD34-positive hematopoietic progenitor/stem cell (and/or other hematopoietic cell described herein) is, in some embodiments, transfected with a polymer nanocapsule or a polymer nanocapsule conjugate as described herein.

As used herein, the term “hyaluronic acid” refers to a polymer capable of binding cell surface receptors for active targeting. Hyaluronic acid is a polysaccharide and one the main components of the extracellular matrix along with collagen

As used herein, the terms “hypoxanthine-guanine phosphoribosyl transferase” or “HPRT” refer to an enzyme involved in purine metabolism encoded by the HPRT1 gene (see, for example, SEQ ID NO: 12). HPRT1 is located on the X chromosome, and thus is present in single copy in males. HPRT1 encodes the transferase that catalyzes the conversion of hypoxanthine to inosine monophosphate and guanine to guanosine monophosphate by transferring the 5-phosphorobosyl group from 5-phosphoribosyl 1-pyrophosphate to the purine. The enzyme functions primarily to salvage purines from degraded DNA for use in renewed purine synthesis.

As used herein, the terms “knock down” or “knockdown” when used in reference to an effect of RNAi on gene expression, means that the level of gene expression is inhibited, or is reduced to a level below that generally observed when examined under substantially the same conditions, but in the absence of RNAi.

As used herein, the terms “knock-out” or “knockout” refer to partial or complete suppression of the expression of an endogenous gene. This is generally accomplished by deleting a portion of the gene or by replacing a portion with a second sequence, but may also be caused by other modifications to the gene such as the introduction of stop codons, the mutation of critical amino acids, the removal of an intron junction, etc. Accordingly, a “knock-out” construct is a nucleic acid sequence, such as a DNA construct, which, when introduced into a cell, results in suppression (partial or complete) of expression of a polypeptide or protein encoded by endogenous DNA in the cell. In some embodiments, a “knockout” includes mutations such as, a point mutation, an insertion, a deletion, a frameshift, or a missense mutation.

As used herein, the terms “multiplicity of infection” or “MOI” means the ratio of agents (e.g. phage or more generally virus, bacteria) to infection targets (e.g. cell). For example, when referring to a group of cells inoculated with virus particles, the multiplicity of infection or MOI is the ratio of the number of virus particles to the number of target cells present in a defined space.

As used herein, the terms “pharmaceutically acceptable carrier or excipient” refers to a carrier or excipient that is useful in preparing a pharmaceutical composition or formulation that is generally safe, non-toxic, and is neither biologically or otherwise undesirable, and includes a carrier or excipient that is acceptable for veterinary use as well as human pharmaceutical use.

As used herein, the terms “positively charged monomer” or “cationic monomer” refer to monomers having a net positive charge, i.e. +1, +2, +3. In some embodiments, the positively charged monomer is a monomer including positively-charged groups. As used herein, the terms “negatively charged monomer” or “anionic monomer” refer to monomers having a net negative charge, i.e. −1, −2, −3. In some embodiments, the negatively charged monomer is a monomer including negatively-charged groups. As used herein, the term “neutral monomer” refers to monomers having a net neutral charge.

As used herein, the term “polymer” is defined as being inclusive of homopolymers, copolymers, interpenetrating networks, and oligomers. Thus, the term polymer may be used interchangeably herein with the term homopolymers, copolymers, interpenetrating polymer networks, etc. The term “homopolymer” is defined as a polymer derived from a single species of monomer. The term “copolymer” is defined as a polymer derived from more than one species of monomer, including copolymers that are obtained by copolymerization of two monomer species, those obtained from three monomers species (“terpolymers”), those obtained from four monomers species (“quaterpolymers”), etc. The term “copolymer” is further defined as being inclusive of random copolymers, alternating copolymers, graft copolymers, and block copolymers. Copolymers, as that term is used generally, include interpenetrating polymer networks. The term “random copolymer” is defined as a copolymer comprising macromolecules in which the probability of finding a given monomeric unit at any given site in the chain is independent of the nature of the adjacent units. In a random copolymer, the sequence distribution of monomeric units follows Bernoullian statistics. The term “alternating copolymer” is defined as a copolymer comprising macromolecules that include two species of monomeric units in alternating sequence.

As used herein, the terms “targeting moiety” or “targeting moieties” and derivatives thereof, refer to a moiety that localizes to or away from a specific locale (e.g., in a subject). For example, in some embodiments, the targeting moiety aids in directing a nanocapsule or polymer nanocapsule conjugate to a specific tissue or location. In some embodiments, the targeting moiety aids in delivering the payloads of the nanocapsule or polymer nanocapsule conjugate, e.g. a ribonucleoprotein complex, to specific tissue sites in vivo or to specific cell types either in vivo or ex vivo. Non-limiting examples include an antibody, an antibody fragment, a peptide, a protein, a polysaccharide, a carbohydrate, a nucleic acid, a vitamin, an aptamer, or a small molecule.

As used herein, the terms “stabilizing moiety” or “stabilizing moieties” and derivatives thereof, refer to a moiety that aids in enhancing the stability of the nanocapsule or the polymer nanocapsule conjugate. For example, in some embodiments, the stabilizing moiety aids in enhancing the stability of the nanocapsule or the polymer nanocapsule conjugate by decreasing aggregation, decreasing opsonization, decreasing phagocytosis, prolonging systemic circulation time, or combinations thereof. Without wishing to be bound by theory, in some embodiments, the stabilizing moiety may also aid in enhancing delivery of the payloads of the nanocapsule or polymer nanocapsule conjugate, e.g. ribonucleoprotein complexes.

As used herein, the phrase “Programmed cell death protein 1” or “PD-1” refers to a cell surface receptor that plays an important role in down-regulating the immune system and promoting self-tolerance by suppressing T cell inflammatory activity. PD-1 is an immune checkpoint and guards against autoimmunity through a dual mechanism of promoting apoptosis (programmed cell death) in antigen-specific T-cells in lymph nodes while simultaneously reducing apoptosis in regulatory T cells (anti-inflammatory, suppressive T cells).

As used herein, the term “reactive group” refers to a functional group that are capable of chemically associating with, interacting with, hybridizing with, hydrogen bonding with, or coupling with a functional group of a different moiety. In some embodiments, a “reaction” between two reactive groups or two reactive functional groups may mean that a covalent linkage is formed between two reactive groups or two reactive functional groups; or may mean that the two reactive groups or two reactive functional groups associate with each other, interact with each other, hybridize to each other, hydrogen bond with each other, etc.

As used herein, the term “subject” refers to a mammal such as a human, mouse or primate. Typically, the mammal is a human (Homo sapiens).

Whenever a group or moiety is described as being “substituted” or “optionally substituted” (or “optionally having” or “optionally comprising”) that group may be unsubstituted or substituted with one or more of the indicated substituents. Likewise, when a group is described as being “substituted or unsubstituted” if substituted, the substituent(s) may be selected from one or more the indicated substituents. If no substituents are indicated, it is meant that the indicated “optionally substituted” or “substituted” group may be substituted with one or more group(s) individually and independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, mercapto, alkylthio, arylthio, cyano, cyanate, halogen, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, an amino, ether, amino (e.g. a mono-substituted amino group or a di-substituted amino group), and protected derivatives thereof.

As used herein, “TCR” refers to a T-cell receptor which is a molecule found on the surface of T cells, or T lymphocytes, that is responsible for recognizing fragments of antigen as peptides bound to major histocompatibility complex (MHC) molecules. The binding between TCR and antigen peptides is of relatively low affinity and is degenerate: that is, many TCRs recognize the same antigen peptide and many antigen peptides are recognized by the same TCR.

As used herein, the term “transferrin” or “transferrins” refers to an iron-binding glycoprotein that is believed to be responsible for iron transport in the body. It is believed that transferring receptors are highly expressed in specific tissues and cells.

As used herein, the terms “treatment,” “treating,” or “treat,” with respect to a specific condition, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment”, as used herein, covers any treatment of a disease or disorder in a subject, particularly in a human, and includes: (a) preventing the disease or disorder from occurring in a subject which may be predisposed to the disease but has not yet been diagnosed as having it; (b) inhibiting the disease or disorder, i.e., arresting its development; and (c) relieving or alleviating the disease or disorder, i.e., causing regression of the disease or disorder and/or relieving one or more disease or disorder symptoms. “Treatment” can also encompass delivery of an agent or administration of a therapy in order to provide for a pharmacologic effect, even in the absence of a disease, disorder or condition. The term “treatment” is used in some embodiments to refer to administration of a compound of the present disclosure to mitigate a disease or a disorder in a host, preferably in a mammalian subject, more preferably in humans. Thus, the term “treatment” can include includes: preventing a disorder from occurring in a host, particularly when the host is predisposed to acquiring the disease but has not yet been diagnosed with the disease; inhibiting the disorder; and/or alleviating or reversing the disorder. As far as the methods of the present disclosure are directed to preventing disorders, it is understood that the term “prevent” does not require that the disease state be completely thwarted. Rather, as used herein, the term preventing refers to the ability of the skilled artisan to identify a population that is susceptible to disorders, such that administration of the compounds of the present disclosure can occur prior to onset of a disease. The term does not mean that the disease state must be completely avoided.

As used herein, the term “zeta potential” refers to the potential difference existing between the surface of a solid particle immersed in a conducting liquid

Polymer Nanocapsules

Provided herein are polymer nanocapsules, such as polymer nanocapsules including a payload. In some embodiments, the “polymer nanocapsules” are compositions of matter which include a polymeric shell and a payload. In some embodiments, the payload is present within a core of the polymer shell. In some embodiments, the payload is encapsulated within the polymeric shell.

Payloads

Any payload may be included within the polymer nanocapsules of the present disclosure. Non-limiting examples of payloads include, but are not limited to, ribonucleoproteins or ribonucleoprotein complexes, siRNA molecules, shRNA molecules, expression vectors, polynucleotides, such as polynucleotides having between about 50 and about 500 base pairs, peptides, enzymes, antibodies, antibody fragments, vectors (e.g. AAV vectors, adeno-associated vectors), etc. While the present disclosure may exemplify ribonucleoprotein complexes as payloads, the polymer nanocapsules (or the conjugates of polymer nanocapsules) disclosed herein are not limited to those including ribonucleoproteins or ribonucleoprotein complexes.

Also provided herein are methods of delivering payloads carried and/or encapsulated by polymer nanocapsules. In some embodiments, methods are provided of delivering payloads including ribonucleoprotein complexes (e.g., Cas9/gRNA) into cells, e.g. host cells, at high efficiencies using the disclosed polymer nanocapsules and/or polymer nanocapsule conjugates. Applicant has discovered that the disclosed polymer nanocapsules and/or polymer nanocapsule conjugates are surprisingly effective in delivering ribonucleoprotein complexes into host cells, such as pluripotent stem cells. In some embodiments, the present disclosure also sets forth a novel strategy of self-assembly and in situ polymerization to imprint ribonucleoprotein complexes into polymer nanocapsules.

A “ribonucleoprotein complex” as provided herein refers to a complex or particle including a nucleoprotein and a ribonucleic acid. A “nucleoprotein” as provided herein refers to a protein capable of binding a nucleic acid (e.g., RNA, DNA). Where the nucleoprotein binds a ribonucleic acid, it is referred to as “ribonucleoprotein.” The interaction between the ribonucleoprotein and the ribonucleic acid is believed to be direct, e.g., by covalent bond, or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In some embodiments, the ribonucleoprotein includes an RNA-binding motif non-covalently bound to the ribonucleic acid. For example, positively charged aromatic amino acid residues (e.g., lysine residues) in the RNA-binding motif may form electrostatic interactions with the negative nucleic acid phosphate backbones of the RNA, thereby forming a ribonucleoprotein complex. Non-limiting examples of ribonucleoproteins include ribosomes, telomerase, RNAseP, hnRNP, CRISPR associated protein 9 (Cas9) and small nuclear RNPs (snRNPs). The ribonucleoprotein may be an enzyme. In embodiments, the ribonucleoprotein is an endonuclease. In some embodiments, the endonuclease is a Cas protein. In some embodiments, the Cas protein is Cas9. In other embodiments, the Cas protein is Cas12. In some embodiments, the Cas12 protein is Cas12a. In other embodiments, the Cas12 protein is Cas12b.

In some embodiments, the CRISPR associated protein is believed to be bound to a ribonucleic acid thereby forming a ribonucleoprotein complex. In some embodiments, the ribonucleic acid is a guide RNA. In some embodiments, the guide RNA includes one or more RNA molecules. In some embodiments, the endonuclease is Cas9 and the ribonucleic acid is a guide RNA. In some embodiments, the endonuclease is Cas12a and the ribonucleic acid is a guide RNA. In some embodiments, the endonuclease is Cas12b and the ribonucleic acid is a guide RNA.

In some embodiments, the gRNA includes a nucleotide sequence complementary to a target site. The complementary nucleotide sequence may mediate binding of the ribonucleoprotein complex to the target site thereby providing the sequence specificity of the ribonucleoprotein complex. In some embodiments, the guide RNA is complementary to a target nucleic acid. In some embodiments, the guide RNA binds a target nucleic acid sequence. In some embodiments, the guide RNA is complementary to a CRISPR nucleic acid sequence. In some embodiments, the complement of the guide RNA has a sequence identity of about 50%, about 55%, about 60%, about 65%, about 70%, about 75%, about 80%, about 85%, about 90%, about 95%, about 96%, about 97%, about 98%, or about 99% to a target nucleic acid. A target nucleic acid sequence as provided herein is a nucleic acid sequence expressed by a cell. In some embodiments, the target nucleic acid sequence is an exogenous nucleic acid sequence. In some embodiments, the target nucleic acid sequence is an endogenous nucleic acid sequence. In some embodiments, the target nucleic acid sequence forms part of a cellular gene. Thus, in some embodiments, the guide RNA is complementary to a cellular gene or fragment thereof. In some embodiments, the guide RNA is about 50%, about 55%, about 60%, about 65%, about 70%, about 75%, about 80%, about 85%, about 90%, about 95%, about 96%, about 97%, about 98% or about 99% to the target nucleic acid sequence. In some embodiments, the guide RNA is about 50%, about 55%, about 60%, about 65%, about 70%, about 75%, about 80%, about 85%, about 90%, about 95%, about 96%, about 97%, about 98% or about 99% complementary to the sequence of a cellular gene. In some embodiments, the guide RNA binds a cellular gene sequence.

In some embodiments, two different versions of Cas9 RNP complexes can be employed: (1) a combination of sgRNA and Cas9 protein, and (2) a combination of crRNA, tracrRNA (two separate strands to form a complete guide RNA) and Cas9 protein. In some embodiments, Cas9, the common component of the two versions, is used as a recombinant protein. In some embodiments, the RNA components in the first version (sgRNA) are typically synthesized using in vitro transcription; while the RNA components in the second version (crRNA and tracrRNA) are chemically synthesized.

In some embodiments, the Cas9 protein is a recombinant S. pyogenes Cas9 nuclease, such as purified from an E. coli strain expressing codon optimized Cas9 and which contains 1 N-terminal nuclear localization sequence (NLS), 2 C-terminal NLSs, and a C-terminal 6-His tag. In some embodiments, the guide RNA is annealed from two parts, tracrRNA (67 nt) from IDT (Cat #1072532) and crRNA (36 nt). Both are chemically modified to increase stability.

Without wishing to be bound by theory, with respect to gene therapies it is generally recognized that targeted integration (in contrast to random integration) of expression cassettes, transgenes, gene fragments or point mutations may offer one or more advantages. Specifically, it is believed that targeted integration may provide improved therapeutic outcomes, may reduce risk of insertional mutagenesis and combinations thereof.

In view of this, the potential advantages of targeting of “safe harbor loci” as integration sites has been described, i.e. Papasavva, P. et al. (2019). Mol Diagn Ther. 23(2): 201-222, the disclosure of which is hereby incorporated by reference herein in its entirety. Safe harbor loci are understood to refer to genomic loci or sites where transgenes or other genetic elements can be safely inserted and/or expressed.

Examples of suitable safe harbor loci include but are not limited to AAV integration site 1 (AAVS1), HPRT locus, albumin locus, hROSA26 locus and chemokine (CC motif) receptor 5 (CCR5) locus. It is recognized that some safe harbor loci may preferably enable insertion of autonomous expression cassettes, for example AAtVSJ and HPRT. Other safe harbor loci may preferably enable expression of transgenes from endogenous control elements, for example hROSA26 and CCR5.

In some embodiments, the payload present in or encapsulated by the nanocapsule or polymer nanocapsule conjugates targets a safe harbor loci. In some embodiments, the payload present in or encapsulated by the nanocapsule or polymer nanocapsule conjugates includes an autonomous expression cassette for insertion in a safe harbor loci. In other embodiments, the payload present in or encapsulated by the nanocapsule or polymer nanocapsule conjugates includes one or more transgenes for insertion in a safe harbor loci. In some embodiments, the safe harbor loci is selected from the group consisting of AAV integration site 1 (AAYVS1), HPRT locus, albumin locus, hROSA26 locus and CCR5 locus. In some preferred embodiments, the safe harbor loci is HPRT. In some embodiments, the nanocapsule or polymer nanocapsule conjugates comprises a payload which targets a safe harbor loci. In some embodiments, the nanocapsule or polymer nanocapsule conjugates comprises a payload which targets the HPRT locus.

In some embodiments, the payload includes a ribonucleoprotein complex. In some embodiments, the ribonucleoprotein complex comprises a guide RNA targeting the CCR5 locus. In some embodiments, the ribonucleoprotein complex comprises a guide RNA targeting the CCR5 locus and an endonuclease. In some embodiments, the ribonucleoprotein complex comprises a guide RNA targeting the CCR5 locus and a Cas protein. In some embodiments, the ribonucleoprotein complex comprises a guide RNA targeting the CCR5 locus and Cas9. In some embodiments, the ribonucleoprotein complex comprises a guide RNA targeting the CCR5 locus and Cas12a. In some embodiments, the ribonucleoprotein complex comprises a guide RNA targeting the CCR5 locus and Cas12b. In some embodiments, the ribonucleoprotein complex comprises a guide RNA having at least 90% sequence identity to that of SEQ ID NO: 1. In some embodiments, the ribonucleoprotein complex comprises a guide RNA having SEQ ID NO: 1. In some embodiments, the ribonucleoprotein complex comprises a guide RNA having at least 90% sequence identity to that of SEQ ID NO: 1 and a Cas protein. In some embodiments, the ribonucleoprotein complex comprises a guide RNA having SEQ ID NO: 1 and a Cas protein.

In some embodiments, the ribonucleoprotein complex comprises a guide RNA targeting the HPRT locus. In some embodiments, the ribonucleoprotein complex comprises a guide RNA targeting the HPRT locus and an endonuclease. In some embodiments, the ribonucleoprotein complex comprises a guide RNA targeting the HPRT locus and a Cas protein. In some embodiments, the ribonucleoprotein complex comprises a guide RNA targeting the HPRT locus and Cas9. In some embodiments, the ribonucleoprotein complex comprises a guide RNA targeting the HPRT locus and Cas12a. In some embodiments, the ribonucleoprotein complex comprises a guide RNA targeting the HPRT locus and Cas12b. In some embodiments, the ribonucleoprotein complex comprises a guide RNA having at least 90% sequence identity to that of SEQ ID NO: 2. In some embodiments, the ribonucleoprotein complex comprises a guide RNA having SEQ ID NO: 2. In some embodiments, the ribonucleoprotein complex comprises a guide RNA having at least 90% sequence identity to that of SEQ ID NO: 2 and a Cas protein. In some embodiments, the ribonucleoprotein complex comprises a guide RNA having SEQ ID NO: 2 and a Cas protein.

In some embodiments, the ribonucleoprotein complex comprises a guide RNA targeting the HBB locus. In some embodiments, the ribonucleoprotein complex comprises a guide RNA targeting the HBB locus and an endonuclease. In some embodiments, the ribonucleoprotein complex comprises a guide RNA targeting the HBB locus and a Cas protein. In some embodiments, the ribonucleoprotein complex comprises a guide RNA targeting the HBB locus and Cas9. In some embodiments, the ribonucleoprotein complex comprises a guide RNA targeting HBB and Cas12a. In some embodiments, the ribonucleoprotein complex comprises a guide RNA targeting the HBB locus and Cas12b. In some embodiments, the ribonucleoprotein complex comprises a guide RNA having at least 90% sequence identity to that of SEQ ID NO: 2. In some embodiments, the ribonucleoprotein complex comprises a guide RNA having SEQ ID NO: 3. In some embodiments, the ribonucleoprotein complex comprises a guide RNA having at least 90% sequence identity to that of SEQ ID NO: 3 and a Cas protein. In some embodiments, the ribonucleoprotein complex comprises a guide RNA having SEQ ID NO: 3 and a Cas protein.

In some embodiments, the ribonucleoprotein complex comprises a guide RNA having at least 90% sequence identity to any one of SEQ ID NOS: 39-54. In some embodiments, the ribonucleoprotein complex comprises a guide RNA having at least 95% sequence identity to any one of SEQ ID NOS: 39-54. In some embodiments, the ribonucleoprotein complex comprises a guide RNA having any one of SEQ ID NOS: 39-54. In some embodiments, the ribonucleoprotein complex comprises a guide RNA having at least 90% sequence identity to any one of SEQ ID NOS: 39-54 and a Cas protein. In some embodiments, the ribonucleoprotein complex comprises a guide RNA having at least 95% sequence identity to any one of SEQ ID NOS: 39-54 and a Cas protein. In some embodiments, the ribonucleoprotein complex comprises a guide RNA having any one of SEQ ID NOS: 39-54 and a Cas protein.

In some embodiments, the ribonucleoprotein complex comprises a guide RNA targeting TCR. In some embodiments, the ribonucleoprotein complex comprises a guide RNA targeting TCR and an endonuclease. In some embodiments, the ribonucleoprotein complex comprises a guide RNA targeting TCR and a Cas protein. In some embodiments, the ribonucleoprotein complex comprises a guide RNA targeting TCR and Cas9. In some embodiments, the ribonucleoprotein complex comprises a guide RNA targeting TCR and Cas12a. In some embodiments, the ribonucleoprotein complex comprises a guide RNA targeting TCR and Cas12b. In some embodiments, the ribonucleoprotein complex comprises a guide RNA having at least 90% sequence identity to that of SEQ ID NO: 4. In some embodiments, the ribonucleoprotein complex comprises a guide RNA having SEQ ID NO: 4. In some embodiments, the ribonucleoprotein complex comprises a guide RNA having at least 90% sequence identity to that of SEQ ID NO: 4 and a Cas protein. In some embodiments, the ribonucleoprotein complex comprises a guide RNA having SEQ ID NO: 4 and a Cas protein.

In some embodiments, the ribonucleoprotein complex comprises a guide RNA targeting PD-1. In some embodiments, the ribonucleoprotein complex comprises a guide RNA targeting PD-1 and an endonuclease. In some embodiments, the ribonucleoprotein complex comprises a guide RNA targeting PD-1 and a Cas protein. In some embodiments, the ribonucleoprotein complex comprises a guide RNA targeting PD-1 and Cas9. In some embodiments, the ribonucleoprotein complex comprises a guide RNA targeting PD-1 and Cas12a. In some embodiments, the ribonucleoprotein complex comprises a guide RNA targeting PD-1 and Cas12b. In some embodiments, the ribonucleoprotein complex comprises a guide RNA having at least 90% sequence identity to that of SEQ ID NO: 5. In some embodiments, the ribonucleoprotein complex comprises a guide RNA having SEQ ID NO: 5. In some embodiments, the ribonucleoprotein complex comprises a guide RNA having at least 90% sequence identity to that of SEQ ID NO: 5 and a Cas protein. In some embodiments, the ribonucleoprotein complex comprises a guide RNA having SEQ ID NO: 5 and a Cas protein.

In some embodiments, the ribonucleoprotein complex comprises a guide RNA targeting BCL11a. In some embodiments, the ribonucleoprotein complex comprises a guide RNA targeting BCL11a and an endonuclease. In some embodiments, the ribonucleoprotein complex comprises a guide RNA targeting BCL11a and a Cas protein. In some embodiments, the ribonucleoprotein complex comprises a guide RNA targeting BCL11a and Cas9. In some embodiments, the ribonucleoprotein complex comprises a guide RNA targeting BCL11a and Cas12a. In some embodiments, the ribonucleoprotein complex comprises a guide RNA targeting BCL11a and Cas12b. In some embodiments, the ribonucleoprotein complex comprises a guide RNA having at least 90% sequence identity to that of SEQ ID NO: 6. In some embodiments, the ribonucleoprotein complex comprises a guide RNA having SEQ ID NO: 6. In some embodiments, the ribonucleoprotein complex comprises a guide RNA having at least 90% sequence identity to that of SEQ ID NO: 6 and a Cas protein. In some embodiments, the ribonucleoprotein complex comprises a guide RNA having SEQ ID NO: 6 and a Cas protein.

In some embodiments, the ribonucleoprotein complex comprises a guide RNA targeting C9orf72. In some embodiments, the ribonucleoprotein complex comprises a guide RNA targeting C9orf72 and an endonuclease. In some embodiments, the ribonucleoprotein complex comprises a guide RNA targeting C9orf72 and a Cas protein. In some embodiments, the ribonucleoprotein complex comprises a guide RNA targeting C9orf72 and Cas9. In some embodiments, the ribonucleoprotein complex comprises a guide RNA targeting C9orf72 and Cas12a. In some embodiments, the ribonucleoprotein complex comprises a guide RNA targeting C9orf72 and Cas12b. In some embodiments, the ribonucleoprotein complex comprises a guide RNA having at least 90% sequence identity to that of SEQ ID NO: 7. In some embodiments, the ribonucleoprotein complex comprises a guide RNA having SEQ ID NO: 7. In some embodiments, the ribonucleoprotein complex comprises a guide RNA having at least 90% sequence identity to that of SEQ ID NO: 7 and a Cas protein. In some embodiments, the ribonucleoprotein complex comprises a guide RNA having SEQ ID NO: 7 and a Cas protein.

In some embodiments, the ribonucleoprotein complex comprises a guide RNA targeting gDrosha1-full. In some embodiments, the ribonucleoprotein complex comprises a guide RNA targeting gDrosha1-full and an endonuclease. In some embodiments, the ribonucleoprotein complex comprises a guide RNA targeting gDrosha1-full and a Cas protein. In some embodiments, the ribonucleoprotein complex comprises a guide RNA targeting gDrosha1-full and Cas9. In some embodiments, the ribonucleoprotein complex comprises a guide RNA targeting gDrosha1-full and Cas12a. In some embodiments, the ribonucleoprotein complex comprises a guide RNA targeting gDrosha1-full and Cas12b. In some embodiments, the ribonucleoprotein complex comprises a guide RNA having at least 90% sequence identity to that of SEQ ID NO: 8. In some embodiments, the ribonucleoprotein complex comprises a guide RNA having SEQ ID NO: 8. In some embodiments, the ribonucleoprotein complex comprises a guide RNA having at least 90% sequence identity to that of SEQ ID NO: 8 and a Cas protein. In some embodiments, the ribonucleoprotein complex comprises a guide RNA having SEQ ID NO: 8 and a Cas protein.

In some embodiments, the payload includes a ribonucleoprotein complex. In some embodiments, the ribonucleoprotein complex comprises a guide RNA targeting gDrosha2-full. In some embodiments, the ribonucleoprotein complex comprises a guide RNA targeting gDrosha2-full and an endonuclease. In some embodiments, the ribonucleoprotein complex comprises a guide RNA targeting gDrosha2-full and a Cas protein. In some embodiments, the ribonucleoprotein complex comprises a guide RNA targeting gDrosha2-full and Cas9. In some embodiments, the ribonucleoprotein complex comprises a guide RNA targeting gDrosha2-full and Cas12a. In some embodiments, the ribonucleoprotein complex comprises a guide RNA targeting gDrosha2-full and Cas12b. In some embodiments, the ribonucleoprotein complex comprises a guide RNA having at least 90% sequence identity to that of SEQ ID NO: 9. In some embodiments, the ribonucleoprotein complex comprises a guide RNA having SEQ ID NO: 9. In some embodiments, the ribonucleoprotein complex comprises a guide RNA having at least 90% sequence identity to that of SEQ ID NO: 9 and a Cas protein. In some embodiments, the ribonucleoprotein complex comprises a guide RNA having SEQ ID NO: 9 and a Cas protein.

In some embodiments, the ribonucleoprotein complex comprises a guide RNA targeting gHPRT4-full. In some embodiments, the ribonucleoprotein complex comprises a guide RNA targeting gHPRT4-full and an endonuclease. In some embodiments, the ribonucleoprotein complex comprises a guide RNA targeting TCR and a Cas protein. In some embodiments, the ribonucleoprotein complex comprises a guide RNA targeting gHPRT4-full and Cas9. In some embodiments, the ribonucleoprotein complex comprises a guide RNA targeting gHPRT4-full and Cas12a. In some embodiments, the ribonucleoprotein complex comprises a guide RNA targeting gHPRT4-full and Cas12b. In some embodiments, the ribonucleoprotein complex comprises a guide RNA having at least 90% sequence identity to that of SEQ ID NO: 10. In some embodiments, the ribonucleoprotein complex comprises a guide RNA having SEQ ID NO: 10. In some embodiments, the ribonucleoprotein complex comprises a guide RNA having at least 90% sequence identity to that of SEQ ID NO: 10 and a Cas protein. In some embodiments, the ribonucleoprotein complex comprises a guide RNA having SEQ ID NO: 10 and a Cas protein.

In some embodiments, the payload includes a ribonucleoprotein complex. In some embodiments, the ribonucleoprotein complex comprises a guide RNA targeting gHPRT3-full. In some embodiments, the ribonucleoprotein complex comprises a guide RNA targeting gHPRT3-full and an endonuclease. In some embodiments, the ribonucleoprotein complex comprises a guide RNA targeting gHPRT3-full and a Cas protein. In some embodiments, the ribonucleoprotein complex comprises a guide RNA targeting gHPRT3-full and Cas9. In some embodiments, the ribonucleoprotein complex comprises a guide RNA targeting gHPRT3-full and Cas12a. In some embodiments, the ribonucleoprotein complex comprises a guide RNA targeting gHPRT3-full and Cas12b. In some embodiments, the ribonucleoprotein complex comprises a guide RNA having at least 90% sequence identity to that of SEQ ID NO: 11. In some embodiments, the ribonucleoprotein complex comprises a guide RNA having SEQ ID NO: 11. In some embodiments, the ribonucleoprotein complex comprises a guide RNA having at least 90% sequence identity to that of SEQ ID NO: 11 and a Cas protein. In some embodiments, the ribonucleoprotein complex comprises a guide RNA having SEQ ID NO: 11 and a Cas protein.

In some embodiments, the ribonucleoprotein complex comprises a guide RNA targeting gWas1. In some embodiments, the ribonucleoprotein complex comprises a guide RNA targeting gWas1 and an endonuclease. In some embodiments, the ribonucleoprotein complex comprises a guide RNA targeting gWas1 and a Cas protein. In some embodiments, the ribonucleoprotein complex comprises a guide RNA targeting gWas1 and Cas9. In some embodiments, the ribonucleoprotein complex comprises a guide RNA targeting gWas1 and Cas12a. In some embodiments, the ribonucleoprotein complex comprises a guide RNA targeting gWas1 and Cas12b. In some embodiments, the ribonucleoprotein complex comprises a guide RNA having at least 90% sequence identity to that of SEQ ID NO: 12. In some embodiments, the ribonucleoprotein complex comprises a guide RNA having SEQ ID NO: 12. In some embodiments, the ribonucleoprotein complex comprises a guide RNA having at least 90% sequence identity to that of SEQ ID NO: 12 and a Cas protein. In some embodiments, the ribonucleoprotein complex comprises a guide RNA having SEQ ID NO: 12 and a Cas protein.

In some embodiments, the ribonucleoprotein complex comprises a guide RNA targeting gHBG1-117. In some embodiments, the ribonucleoprotein complex comprises a guide RNA targeting gHBG1-117 and an endonuclease. In some embodiments, the ribonucleoprotein complex comprises a guide RNA targeting gHBG1-117 and a Cas protein. In some embodiments, the ribonucleoprotein complex comprises a guide RNA targeting gHBG1-117 and Cas9. In some embodiments, the ribonucleoprotein complex comprises a guide RNA targeting gHBG1-117 and Cas12a. In some embodiments, the ribonucleoprotein complex comprises a guide RNA targeting gHBG-117 and Cas12b. In some embodiments, the ribonucleoprotein complex comprises a guide RNA having at least 90% sequence identity to that of SEQ ID NO: 13. In some embodiments, the ribonucleoprotein complex comprises a guide RNA having SEQ ID NO: 13. In some embodiments, the ribonucleoprotein complex comprises a guide RNA having at least 90% sequence identity to that of SEQ ID NO: 13 and a Cas protein. In some embodiments, the ribonucleoprotein complex comprises a guide RNA having SEQ ID NO: 13 and a Cas protein.

In some embodiments, the ribonucleoprotein complex comprises a guide RNA targeting gHBG2-114. In some embodiments, the ribonucleoprotein complex comprises a guide RNA targeting gHBG2-114 and an endonuclease. In some embodiments, the ribonucleoprotein complex comprises a guide RNA targeting gHBG2-114 and a Cas protein. In some embodiments, the ribonucleoprotein complex comprises a guide RNA targeting gHBG2-114 and Cas9. In some embodiments, the ribonucleoprotein complex comprises a guide RNA targeting gHBG2-114 and Cas12a. In some embodiments, the ribonucleoprotein complex comprises a guide RNA targeting gHBG2-114 and Cas12b. In some embodiments, the ribonucleoprotein complex comprises a guide RNA having at least 90% sequence identity to that of SEQ ID NO: 14. In some embodiments, the ribonucleoprotein complex comprises a guide RNA having SEQ ID NO: 14. In some embodiments, the ribonucleoprotein complex comprises a guide RNA having at least 90% sequence identity to that of SEQ ID NO: 14 and a Cas protein. In some embodiments, the ribonucleoprotein complex comprises a guide RNA having SEQ ID NO: 14 and a Cas protein.

In some embodiments, the ribonucleoprotein complex comprises a guide RNA targeting gHBB2. In some embodiments, the ribonucleoprotein complex comprises a guide RNA targeting gHBB2 and an endonuclease. In some embodiments, the ribonucleoprotein complex comprises a guide RNA targeting gHBB2 and a Cas protein. In some embodiments, the ribonucleoprotein complex comprises a guide RNA targeting gHBB2 and Cas9. In some embodiments, the ribonucleoprotein complex comprises a guide RNA targeting gHBB2 and Cas12a. In some embodiments, the ribonucleoprotein complex comprises a guide RNA targeting gHBB2 and Cas12b. In some embodiments, the ribonucleoprotein complex comprises a guide RNA having at least 90% sequence identity to that of SEQ ID NO: 15. In some embodiments, the ribonucleoprotein complex comprises a guide RNA having SEQ ID NO: 15. In some embodiments, the ribonucleoprotein complex comprises a guide RNA having at least 90% sequence identity to that of SEQ ID NO: 15 and a Cas protein. In some embodiments, the ribonucleoprotein complex comprises a guide RNA having SEQ ID NO: 15 and a Cas protein.

In some embodiments, the ribonucleoprotein complex comprises a guide RNA targeting gHBG12a1. In some embodiments, the ribonucleoprotein complex comprises a guide RNA targeting gHBG12a1 and an endonuclease. In some embodiments, the ribonucleoprotein complex comprises a guide RNA targeting gHBG12a1 and a Cas protein. In some embodiments, the ribonucleoprotein complex comprises a guide RNA targeting gHBG12a1 and Cas9. In some embodiments, the ribonucleoprotein complex comprises a guide RNA targeting gHBG12a1 and Cas12a. In some embodiments, the ribonucleoprotein complex comprises a guide RNA targeting gHBG12a1 and Cas12b. In some embodiments, the ribonucleoprotein complex comprises a guide RNA having at least 90% sequence identity to that of SEQ ID NO: 16. In some embodiments, the ribonucleoprotein complex comprises a guide RNA having SEQ ID NO: 16. In some embodiments, the ribonucleoprotein complex comprises a guide RNA having at least 90% sequence identity to that of SEQ ID NO: 16 and a Cas protein. In some embodiments, the ribonucleoprotein complex comprises a guide RNA having SEQ ID NO: 16 and a Cas protein.

In some embodiments, the ribonucleoprotein complex comprises a guide RNA targeting gHBG12a2. In some embodiments, the ribonucleoprotein complex comprises a guide RNA targeting gHBG12a2 and an endonuclease. In some embodiments, the ribonucleoprotein complex comprises a guide RNA targeting gHBG12a2 and a Cas protein. In some embodiments, the ribonucleoprotein complex comprises a guide RNA targeting gHBG12a2 and Cas9. In some embodiments, the ribonucleoprotein complex comprises a guide RNA targeting gHBG12a2 and Cas12a. In some embodiments, the ribonucleoprotein complex comprises a guide RNA targeting gHBG12a2 and Cas12b. In some embodiments, the ribonucleoprotein complex comprises a guide RNA having at least 90% sequence identity to that of SEQ ID NO: 17. In some embodiments, the ribonucleoprotein complex comprises a guide RNA having SEQ ID NO: 17. In some embodiments, the ribonucleoprotein complex comprises a guide RNA having at least 90% sequence identity to that of SEQ ID NO: 17 and a Cas protein. In some embodiments, the ribonucleoprotein complex comprises a guide RNA having SEQ ID NO: 17 and a Cas protein.

In some embodiments, the payload includes a ribonucleoprotein complex. In some embodiments, the ribonucleoprotein complex comprises a guide RNA targeting gHBG12a3. In some embodiments, the ribonucleoprotein complex comprises a guide RNA targeting gHBG12a3 and an endonuclease. In some embodiments, the ribonucleoprotein complex comprises a guide RNA targeting gHBG12a3 and a Cas protein. In some embodiments, the ribonucleoprotein complex comprises a guide RNA targeting gHBG12a3 and Cas9. In some embodiments, the ribonucleoprotein complex comprises a guide RNA targeting gHBG12a3 and Cas12a. In some embodiments, the ribonucleoprotein complex comprises a guide RNA targeting gHBG12a3 and Cas12b. In some embodiments, the ribonucleoprotein complex comprises a guide RNA having at least 90% sequence identity to that of SEQ ID NO: 18. In some embodiments, the ribonucleoprotein complex comprises a guide RNA having SEQ ID NO: 18. In some embodiments, the ribonucleoprotein complex comprises a guide RNA having at least 90% sequence identity to that of SEQ ID NO: 18 and a Cas protein. In some embodiments, the ribonucleoprotein complex comprises a guide RNA having SEQ ID NO: 18 and a Cas protein.

In some embodiments, the ribonucleoprotein complex comprises a guide RNA targeting gHBG12a4. In some embodiments, the ribonucleoprotein complex comprises a guide RNA targeting gHBG12a4 and an endonuclease. In some embodiments, the ribonucleoprotein complex comprises a guide RNA targeting gHBG12a4 and a Cas protein. In some embodiments, the ribonucleoprotein complex comprises a guide RNA targeting gHBG12a4 and Cas9. In some embodiments, the ribonucleoprotein complex comprises a guide RNA targeting gHBG12a4 and Cas12a. In some embodiments, the ribonucleoprotein complex comprises a guide RNA targeting gHBG12a4 and Cas12b. In some embodiments, the ribonucleoprotein complex comprises a guide RNA having at least 90% sequence identity to that of SEQ ID NO: 19. In some embodiments, the ribonucleoprotein complex comprises a guide RNA having SEQ ID NO: 19. In some embodiments, the ribonucleoprotein complex comprises a guide RNA having at least 90% sequence identity to that of SEQ ID NO: 19 and a Cas protein. In some embodiments, the ribonucleoprotein complex comprises a guide RNA having SEQ ID NO: 19 and a Cas protein.

In some embodiments, the payload includes a ribonucleoprotein complex. In some embodiments, the ribonucleoprotein complex comprises a guide RNA targeting gHBB12a1. In some embodiments, the ribonucleoprotein complex comprises a guide RNA targeting gHBB12a1 and an endonuclease. In some embodiments, the ribonucleoprotein complex comprises a guide RNA targeting gHBB12a1 and a Cas protein. In some embodiments, the ribonucleoprotein complex comprises a guide RNA targeting gHBB12a1 and Cas9. In some embodiments, the ribonucleoprotein complex comprises a guide RNA targeting gHBB12a1 and Cas12a. In some embodiments, the ribonucleoprotein complex comprises a guide RNA targeting gHBB12a1 and Cas12b. In some embodiments, the ribonucleoprotein complex comprises a guide RNA having at least 90% sequence identity to that of SEQ ID NO: 20. In some embodiments, the ribonucleoprotein complex comprises a guide RNA having SEQ ID NO: 20. In some embodiments, the ribonucleoprotein complex comprises a guide RNA having at least 90% sequence identity to that of SEQ ID NO: 20 and a Cas protein. In some embodiments, the ribonucleoprotein complex comprises a guide RNA having SEQ ID NO: 20 and a Cas protein.

In some embodiments, the ribonucleoprotein complex comprises a guide RNA targeting gHBB12a2. In some embodiments, the ribonucleoprotein complex comprises a guide RNA targeting gHBB12a2 and an endonuclease. In some embodiments, the ribonucleoprotein complex comprises a guide RNA targeting gHBB12a2 and a Cas protein. In some embodiments, the ribonucleoprotein complex comprises a guide RNA targeting gHBB12a2 and Cas9. In some embodiments, the ribonucleoprotein complex comprises a guide RNA targeting gHBB12a2 and Cas12a. In some embodiments, the ribonucleoprotein complex comprises a guide RNA targeting gHBB12a2 and Cas12b. In some embodiments, the ribonucleoprotein complex comprises a guide RNA having at least 90% sequence identity to that of SEQ ID NO: 21. In some embodiments, the ribonucleoprotein complex comprises a guide RNA having SEQ ID NO: 21. In some embodiments, the ribonucleoprotein complex comprises a guide RNA having at least 90% sequence identity to that of SEQ ID NO: 21 and a Cas protein. In some embodiments, the ribonucleoprotein complex comprises a guide RNA having SEQ ID NO: 21 and a Cas protein.

In some embodiments, the ribonucleoprotein complex comprises a guide RNA targeting gHPRT12a1. In some embodiments, the ribonucleoprotein complex comprises a guide RNA targeting gHPRT12a1 and an endonuclease. In some embodiments, the ribonucleoprotein complex comprises a guide RNA targeting gHPRT12a1 and a Cas protein. In some embodiments, the ribonucleoprotein complex comprises a guide RNA targeting gHPRT12a1 and Cas9. In some embodiments, the ribonucleoprotein complex comprises a guide RNA targeting gHPRT12a1 and Cas12a. In some embodiments, the ribonucleoprotein complex comprises a guide RNA targeting gHPRT12a1 and Cas12b. In some embodiments, the ribonucleoprotein complex comprises a guide RNA having at least 90% sequence identity to that of SEQ ID NO: 22. In some embodiments, the ribonucleoprotein complex comprises a guide RNA having SEQ ID NO: 22. In some embodiments, the ribonucleoprotein complex comprises a guide RNA having at least 90% sequence identity to that of SEQ ID NO: 22 and a Cas protein. In some embodiments, the ribonucleoprotein complex comprises a guide RNA having SEQ ID NO: 22 and a Cas protein.

In some embodiments, the ribonucleoprotein complex comprises a guide RNA targeting gHPRT12a2. In some embodiments, the ribonucleoprotein complex comprises a guide RNA targeting gHPRT12a2 and an endonuclease. In some embodiments, the ribonucleoprotein complex comprises a guide RNA targeting gHPRT12a2 and a Cas protein. In some embodiments, the ribonucleoprotein complex comprises a guide RNA targeting gHPRT12a2 and Cas9. In some embodiments, the ribonucleoprotein complex comprises a guide RNA targeting gHPRT12a2 and Cas12a. In some embodiments, the ribonucleoprotein complex comprises a guide RNA targeting gHPRT12a2 and Cas12b. In some embodiments, the ribonucleoprotein complex comprises a guide RNA having at least 90% sequence identity to that of SEQ ID NO: 23. In some embodiments, the ribonucleoprotein complex comprises a guide RNA having SEQ ID NO: 23. In some embodiments, the ribonucleoprotein complex comprises a guide RNA having at least 90% sequence identity to that of SEQ ID NO: 23 and a Cas protein. In some embodiments, the ribonucleoprotein complex comprises a guide RNA having SEQ ID NO: 23 and a Cas protein.

In some embodiments, the ribonucleoprotein complex comprises a guide RNA targeting Gamma-globin promoter. In some embodiments, the ribonucleoprotein complex comprises a guide RNA targeting Gamma-globin promoter and an endonuclease. In some embodiments, the ribonucleoprotein complex comprises a guide RNA targeting Gamma-globin promoter and a Cas protein. In some embodiments, the ribonucleoprotein complex comprises a guide RNA targeting Gamma-globin promoter and Cas9. In some embodiments, the ribonucleoprotein complex comprises a guide RNA targeting Gamma-globin promoter and Cas12a. In some embodiments, the ribonucleoprotein complex comprises a guide RNA targeting Gamma-globin promoter and Cas12b.

Non-limiting examples of guide RNAs which may be incorporated within any ribonucleoprotein complex are set forth below:

| Target gene | Sequence for chemically modified RNA | SEQ ID NO: |
|---|---|---|
| CCR5 | /AltR1/rGrC rUrGrU rGrUrU rUrGrC rGrUrC | 1 |
| | rUrCrU rCrCrC rGrUrU rUrUrA rGrArG | |
| | rCrUrA rUrGrC rU/AltR2/ | |
| HPRT | /AltR1/rGrCrU rGrUrG rUrUrU rGrCrG | 2 |
| | rUrCrU rCrUrC rCrCrG rUrUrU rUrArG | |
| | rArGrC rUrArU rGrCrU /AltR2/ | |
| HBB | /AltR1/rCrUrU rGrCrC rCrCrA rCrArG rGrGrC | 3 |
| | rArGrU rArArG rUrUrU rUrArG rArGrC | |
| | rUrArU rGrCrU /AltR2/ | |
| TCR | /AltR1/rGrArG rArArU rCrArA rArArU | 4 |
| | rCrGrG rUrGrA rArUrG rUrUrU rUrArG | |
| | rArGrC rUrArU rGrCrU /AltR2/ | |
| PD-1 | /AltR1/rUrG rArCrG rUrUrA rCrCrU rCrGrU | 5 |
| | rGrCrG rGrCrC rGrUrU rUrUrA rGrArG | |
| | rCrUrA rUrGrC rU/AltR2/ | |
| BCL11a | /AltR1/rGrA rUrArA rArCrA rArUrC rGrUrC | 6 |
| | rArUrC rCrUrC rGrUrU rUrUrA rGrArG | |
| | rCrUrA rUrGrC rU/AltR2/ | |
| C9orf72 | /AltR1/rUrA rCrUrA rCrArA rCrGrG rArArC | 7 |
| | rArGrC rCrArC rGrUrU rUrUrA rGrArG | |
| | rCrUrA rUrGrC rU/AltR2/ | |
| gDrosha1-full | mU*mG*mC* rUrGrC rGrGrC rArUrG rArCrU | 8 |
| | rGrGrA rGrGrG rUrUrU rUrArG rArGrC | |
| | rUrArG rArArA rUrArG rCrArA rGrUrU | |
| | rArArA rArUrA rArGrG rCrUrA rGrUrC | |
| | rCrGrU rUrArU rCrArA rCrUrU rGrArA | |
| | rArArA rGrUrG rGrCrA rCrCrG rArGrU | |
| | rCrGrG rUrGrC mU*mU*mU* rU | |
| gDrosha2-full | mU*mG*mA* rGrUrG rCrArU rCrGrA rArUrC | 9 |
| | rCrArC rArCrG rUrUrU rUrArG rArGrC | |
| | rUrArG rArArA rUrArG rCrArA rGrUrU | |
| | rArArA rArUrA rArGrG rCrUrA rGrUrC | |
| | rCrGrU rUrArU rCrArA rCrUrU rGrArA | |
| | rArArA rGrUrG rGrCrA rCrCrG rArGrU | |
| | rCrGrG rUrGrC mU*mU*mU* rU | |
| gHPRT4-full | mG*mC*mG* rGrGrU rCrGrC rCrArU rArArC | 10 |
| | rGrGrA rGrCrG rUrUrU rUrArG rArGrC | |
| | rUrArG rArArA rUrArG rCrArA rGrUrU | |
| | rArArA rArUrA rArGrG rCrUrA rGrUrC | |
| | rCrGrU rUrArU rCrArA rCrUrU rGrArA | |
| | rArArA rGrUrG rGrCrA rCrCrG rArGrU | |
| | rCrGrG rUrGrC mU*mU*mU* rU | |
| gHPRT3-full | mG*mG*mC* rUrUrA rUrArU rCrCrA rArCrA | 11 |
| | rCrUrU rCrGrG rUrUrU rUrArG rArGrC | |
| | rUrArG rArArA rUrArG rCrArA rGrUrU | |
| | rArArA rArUrA rArGrG rCrUrA rGrUrC | |
| | rCrGrU rUrArU rCrArA rCrUrU rGrArA | |
| | rArArA rGrUrG rGrCrA rCrCrG rArGrU | |
| | rCrGrG rUrGrC mU*mU*mU* rU | |
| gWas1 | mC*mG*mC* rCrArU rGrGrG rUrCrA rUrUrU | 12 |
| | rCrArC rArGrG rUrUrU rUrArG rArGrC | |
| | rUrArG rArArA rUrArG rCrArA rGrUrU | |
| | rArArA rArUrA rArGrG rCrUrA rGrUrC | |
| | rCrGrU rUrArU rCrArA rCrUrU rGrArA | |
| | rArArA rGrUrG rGrCrA rCrCrG rArGrU | |
| | rCrGrG rUrGrC mU*mU*mU* rU | |
| gHBG1-117 | mC*mU*mU* rGrUrC rArArG rGrCrU rArUrU | 13 |
| | rGrGrU rCrArG rUrUrU rUrArG rArGrC | |
| | rUrArG rArArA rUrArG rCrArA rGrUrU | |
| | rArArA rArUrA rArGrG rCrUrA rGrUrC | |
| | rCrGrU rUrArU rCrArA rCrUrU rGrArA | |
| | rArArA rGrUrG rGrCrA rCrCrG rArGrU | |
| | rCrGrG rUrGrC mU*mU*mU* rU | |
| gHBG2-114 | mG*mU*mU* rUrGrC rCrUrU rGrUrC rArArG | 14 |
| | rGrCrU rArUrG rUrUrU rUrArG rArGrC | |
| | rUrArG rArArA rUrArG rCrArA rGrUrU | |
| | rArArA rArUrA rArGrG rCrUrA rGrUrC | |
| | rCrGrU rUrArU rCrArA rCrUrU rGrArA | |
| | rArArA rGrUrG rGrCrA rCrCrG rArGrU | |
| | rCrGrG rUrGrC mU*mU*mU* rU | |
| gHBB2 | mG*mU*mA* rArCrG rGrCrA rGrArC rUrUrC | 15 |
| | rUrCrC rUrCrG rUrUrU rUrArG rArGrC | |
| | rUrArG rArArA rUrArG rCrArA rGrUrU | |
| | rArArA rArUrA rArGrG rCrUrA rGrUrC | |
| | rCrGrU rUrArU rCrArA rCrUrU rGrArA | |
| | rArArA rGrUrG rGrCrA rCrCrG rArGrU | |
| | rCrGrG rUrGrC mU*mU*mU* rU | |

*= phosphorothioate linkage

Additional non-limiting examples of guide RNAs which may be incorporated within any ribonucleoprotein complex, such as those including a Cas12a protein, are set forth below:

| Target gene | Sequence for chemically modified RNA | SEQ ID NO: |
|---|---|---|
| gHBG12a1 | rUrA rArUrU rUrCrU rArCrU rCrUrU rGrUrA | 16 |
| | rGrArU rCrCrU rUrGrU rCrArA rGrGrC | |
| | rUrArU rUrGrG rUrCrA rArGrG | |
| gHBG12a2 | rUrA rArUrU rUrCrU rArCrU rCrUrU rGrUrA | 17 |
| | rGrArU rGrCrC rArGrG rGrArC rCrGrU | |
| | rUrUrC rArGrA rCrArG rArUrA | |
| gHBG12a3 | rUrA rArUrU rUrCrU rArCrU rCrUrU rGrUrA | 18 |
| | rGrArU rArGrA rCrArG rArUrA rUrUrU | |
| | rGrCrA rUrUrG rArGrA rUrArG | |
| gHBG12a4 | rUrA rArUrU rUrCrU rArCrU rCrUrU rGrUrA | 19 |
| | rGrArU rCrArU rUrGrA rGrArU rArGrU | |
| | rGrUrG rGrGrG rArArG rGrGrG | |
| gHBB12a1 | rUrA rArUrU rUrCrU rArCrU rCrUrU rGrUrA | 20 |
| | rGrArU rCrUrU rCrUrG rArCrA rCrArA | |
| | rCrUrG rUrGrU rUrCrA rCrUrA | |
| gHBB12a2 | rUrA rArUrU rUrCrU rArCrU rCrUrU rGrUrA | 21 |
| | rGrArU rArGrG rUrUrG rCrUrA rGrUrG | |
| | rArArC rArCrA rGrUrU rGrUrG | |
| gHPRT12a1 | rUrA rArUrU rUrCrU rArCrU rCrUrU rGrUrA | 22 |
| | rGrArU rArGrA rUrCrU rUrArC rUrUrA | |
| | rCrCrU rGrUrC rCrArU rArArU | |
| gHPRT12a2 | rUrA rArUrU rUrCrU rArCrU rCrUrU rGrUrA | 23 |
| | rGrArU rGrCrA rUrArC rCrUrA rArUrC | |
| | rArUrU rArUrG rCrUrG rArGrG | |

Yet other non-limiting examples of target sequences for guide RNAs are set forth below:

| | | SEQ ID |
|---|---|---|
| Target gene | Sequence | NO: |
| Gamma-globin | CTTGTCAAGGCTATTGGTCAAGG | 24 |
| promoter | | |
| Gamma-globin | CAAGGCTATTGGTCAAGGCAAGG | 25 |
| promoter | | |
| Gamma-globin | GCTATTGGTCAAGGCAAGGCCTG | 26 |
| promoter | | |
| Gamma-globin | TGGTCAAGTTTGCCTTGTCAAGG | 27 |
| promoter | | |
| Gamma-globin | CTTGACCAATAGCCTTGACAAGG | 28 |
| promoter | | |
| Gamma-globin | TTTGCCTTGTCAAGGCTATTGGTCAAGG | 29 |
| promoter | | |
| HPRT | GTTATGGCGACCCGCAGCCC | 30 |
| HPRT | GCGGGTCGCCATAACGGAGC | 31 |
| HPRT | CGTGACGTAAAGCCGAACCC | 32 |
| HPRT | GGCACGGAAAGCGACCACCT | 33 |
| HPRT | CATGCGTATTTGACACACGA | 34 |
| HPRT | GCTAAGTGCTAGAGTTACGG | 35 |
| HPRT | GAGGCGCGGGATCCGCAGTG | 36 |
| HPRT | TTATTACTGTTCCCCGCCAG | 37 |

Additional target genes for gRNAs could include BCL11a for sickle cell disease, PD-1 for cancer therapy, and/or C9orf72 for amyotrophic lateral sclerosis (ALS).

In some embodiments, the polymer nanocapsules (or conjugates described herein) are utilized to knockout HPRT. For example, isolated cells may be treated with a HPRT-targeted CRISPR/Cas9 RNP, such as those included within the polymer nanocapsules disclosed herein. In some embodiments, the nanocapsule includes a ribonucleoprotein complex which includes a gRNA molecule targeting a sequence within the human hypoxanthine phosphoribosyl transferase (HPRT) gene. In some embodiments, the nanocapsule includes a ribonucleoprotein complex which includes a gRNA molecule targeting a sequence within Chromosome X of a human at a location ranging from about 134460145 to about 134500668. In some embodiments, the targeted sequence within the location ranging from about 134460145 to about 134500668 of Chromosome X ranges from about 12 to about 28 consecutive base pairs in length. In some embodiments, the targeted sequence within the location ranging from about 134460145 to about 134500668 of Chromosome X ranges from about 14 to about 26 consecutive base pairs in length. In some embodiments, the targeted sequence within the location ranging from about 134460145 to about 134500668 of Chromosome X ranges from about 16 to about 24 consecutive base pairs in length. In some embodiments, the targeted sequence within the location ranging from about 134460145 to about 134500668 of Chromosome X ranges from about 18 to about 22 consecutive base pairs in length.

In some embodiments, suitable targets within the HPRT gene include those having 90% identity to any of SEQ ID NOS: 30-37. In other embodiments, suitable targets within the HPRT gene include those having 95% identity to any of SEQ ID NOS: 30-37. In other embodiments, suitable targets within the HPRT gene include those having 96% identity to any of SEQ ID NOS: 30-37. In other embodiments, suitable targets within the HPRT gene include those having 97% identity to any of SEQ ID NOS: 30-37. In other embodiments, suitable targets within the HPRT gene include those having 98% identity to any of SEQ ID NOS: 30-37. In other embodiments, suitable targets within the HPRT gene include those having 99% identity to any of SEQ ID NOS: 30-37. In other embodiments, suitable targets within the HPRT gene include those having any of SEQ ID NOS: 30-37.

Polymeric Shell

Different combinations of monomers and crosslinkers may be used to form a polymeric shell by in situ polymerization, and thus encapsulate the payloads, e.g. ribonucleoprotein complexes. In some embodiments, the polymer nanocapsules of the present disclosure include at least one positively charged monomer, a neutral monomer, and a crosslinker (e.g. a degradable or erodible crosslinker). In other embodiments, the polymer nanocapsules of the present disclosure include two positively charged monomers, a neutral monomer, and a crosslinker. Non-limiting examples of suitable positive monomers, neutral monomers, and crosslinkers are disclosed below.

In some embodiments, suitable positively charged monomers for forming the polymeric shell of the polymer nanocapsules of the present disclosure have the structures of any of Formulas (IA) and (IB):

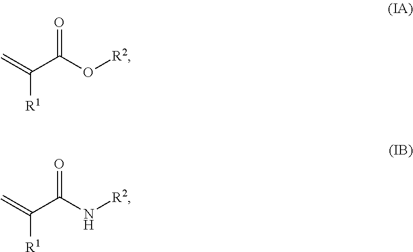

wherein

R¹ is H or a substituted or unsubstituted C₁-C₆ alkyl group;

R² is —(CH₂)ₘ—NR³R⁴R⁵, where m is an integer from 1 to 5;

R³ is H, an unsubstituted C₁-C₆ alkyl group, or a C₁-C₆ alkyl group substituted with NR⁶R⁷, where R⁶ and R⁷ are independently selected from H, or an unsubstituted C₁-C₆ alkyl group, or a C₁-C₆ alkyl group substituted with amino or a C₁-C₆ alkyl substituted with NR⁸R⁹, wherein R⁸ and R⁹ are independently selected from H, an unsubstituted C₁-C₆ alkyl group, or a C₁-C₆ alkyl substituted with amino;

R⁴ is H, an unsubstituted C₁-C₆ alkyl group, or a C₁-C₆ alkyl group substituted with amino, or a C₁-C₆alkyl group substituted with NR¹⁰R¹¹, where R¹⁰ and R¹¹ are independently selected from H, an unsubstituted C₁-C₆ alkyl group, a C₁-C₆ alkyl group substituted with amino, or a C₁-C₆ alkyl group substituted with NR¹²R¹³, where R¹² and R¹³ are independently selected from H, an unsubstituted C₁-C₆ alkyl group, or a C₁-C₆ alkyl group substituted with amino;

or where R¹ and R⁴, may be taken together to form a 5- to 7-membered heterocycloalkyl ring; and

R⁵ is a lone pair of electrons or an unsubstituted C₁-C₆ alkyl group.

In other embodiments, suitable positively charged monomers for forming the polymer nanocapsules of the present disclosure have the structures of any of Formulas (IC) or (ID):

where R² is as defined above.

In some embodiments, the positively charged monomer is selected from the group consisting of:

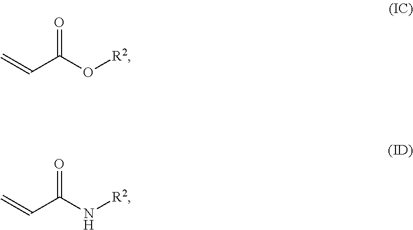

N-(3-((4-((3-aminopropyl)amino)butyl)amino) propyl)acrylamide,
N-(3-((4-((3-aminopropyl)amino)butyl)amino) propyl)methacrylamide,
N-(3-((4-aminobutyl)amino)propyl)acrylamide,
N-(3-((4-aminobutyl)amino)propyl)methacrylamide,
N-(2-((2-aminoethyl)(methyl)amino)ethyl)acrylamide,
N-(2-((2-aminoethyl)(methyl)amino)ethyl)methacrylamide,
N-(piperazin-1-ylmethyl)acrylamide,
N-(piperazin-1-ylmethyl)methacrylamide,
N-(2-(bis(2-aminoethyl)amino)ethyl)acrylamide,
N-(2-(bis(2-aminoethyl)amino)ethyl)methacrylamide,
N-(3-Aminopropyl) methacrylamide hydrochloride,
2-(dimethylamino)ethyl acrylate,
Dimethylamino ethyl methacrylate,
(3-Acrylamidopropyl) trimethylammonium hydrochloride,
2-aminoethyl methacrylate,
3-(Dimethylamino)propyl acrylate, and
N-[3-(Dimethylamino)propyl]methacrylamid.

In some embodiments, the positive monomer is selected from the group consisting of N-(3-((4-((3-aminopropyl)amino)butyl)amino)propyl)acrylamide, 2-(dimethylamino)ethyl acrylate, and any combination thereof.

In some embodiments, suitable crosslinkers for forming the polymer nanocapsules of the present disclosure have the structure of Formula (IE):

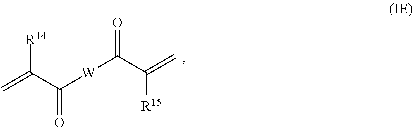

wherein R¹⁴ and R¹¹ are independently H, or a substituted or unsubstituted C₁-C₆ alkyl group, and

W is —N(H)—R¹⁶—N(H)— or —[O—CH₂—C(H)(OH)—CH₂]ₙ—O—, where R¹⁶ is a substituted or unsubstituted C₁-C₆ alkylene.

In some embodiments, the crosslinkers are selected from 1,3-glycerol dimethacrylate, N,N′-Methylenebisacrylamide, and/or Glycerol 1,3-diglycerolate diacrylate.

In some embodiments, suitable neutral monomers for forming the polymer nanocapsules of the present disclosure have the structure of Formula (IF):

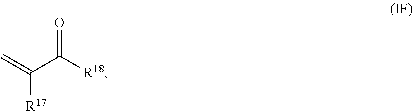

wherein R¹⁷ is H or an unsubstituted C₁-C₆ alkyl group, and

R¹⁸ is amino or amino substituted with a hydroxy substituted alkyl, or OR¹⁹, where R¹⁹ is hydroxy alkyl substituent.

In some embodiments, the neutral monomers are selected from N-(1,3-dihydroxy-2-(hydroxymethyl)propan-2-yl)acrylamide, acrylamide, N-(hydroxymethyl)acrylamide, 2-hydroxyethyl acrylate, and/or 2-hydroxyethyl methacrylate.

In some embodiments, the polymer nanocapsules may synthesized through an in-situ polymerization technique. In some embodiments, the polymerization may start with at least one positively charged monomer (e.g. N-(3-((4-((3-aminopropyl)amino)butyl)amino)propyl)acrylamide and/or 2-(dimethylamino)ethyl acrylate), a cross-linker (e.g. 1,3-glycerol dimethacrylate), and a neutral monomer (e.g. acrylamide). The monomers may then be enriched around the surface of the payload, e.g. negatively charged ribonucleoprotein complexes, through electrostatic interactions and/or hydrogen bonding. Without wishing to be bound by any particular theory, it is believed that different crosslinkers may be used to form copolymer coatings having tunable compositions, structure, surface properties, and functionalities. It is also believed that the crosslinked polymer shell provided by the aforementioned polymerization provides protection to the payloads, e.g. ribonucleoprotein complexes, from, for example, enzymatic degradation, temperature dissociation and serum inactivation.

By way of another example, synthesis of the polymer nanocapsules utilizes electrostatic interactions present around the surface of a payload, e.g. the negatively charged ribonucleoprotein complexes. For example, the monomers may self-assemble along the surface of the negatively charged ribonucleoprotein complexes through electrostatic interactions and/or hydrogen bonding. After the initial interactions, subsequent room-temperature polymerization in an aqueous solution may take place with a crosslinker and positive and neutral monomers. During the room temperature polymerization, it is believed that each ribonucleoprotein complex is “wrapped” in a thin shell of a polymer network. It is believed that such a crosslinked shell (i.e. the polymer shell) serves to protect ribonucleoprotein complexes, now present within a core of the polymer nanocapsule, from hydrolysis, etc. Additional moieties may be added to the formed polymeric shell for targeting and/or for increasing water solubility and/or charge, as described further herein.

In some embodiments, the polymer nanocapsules are synthesized using an encapsulation buffer, such as one having a pH ranging from between about 6 and about 7.5. In other embodiments, the pH of the encapsulation buffer ranges from between about 6 and about 7. In yet other embodiments, the pH of the encapsulation buffer is about 6.7. In some embodiments, the encapsulation buffer comprises 4-(2-hydroxyethyl)-1-piperazineethanesulfonic acid (HEPES), NaCl, and/or MgCl₂. In some embodiments, HEPES is present in amount ranging from between about 10 to about 50 mM. In other embodiments, HEPES is present in an amount of about 20 mM. In some embodiments, NaCl is present in amount ranging from between about 50 to about 150 mM. In other embodiments, NaCl is present in an amount of about 100 mM. In some embodiments, MgCl₂ is present in amount ranging from between about 1 to about 10 mM. In other embodiments, MgCl₂ is present in an amount of about 5 mM.

In some embodiments, an amount of positive monomer ranges from between about 20% to about 65% by total weight of the polymer shell of the polymer nanocapsule. In other embodiments, an amount of positive monomer ranges from between about 25% to about 60% by total weight of the polymer shell of the polymer nanocapsule. In yet other embodiments, an amount of positive monomer ranges from between about 25% to about 55% by total weight of the polymer shell of the polymer nanocapsule. In further embodiments, an amount of positive monomer ranges from between about 30% to about 50% by total weight of the polymer shell of the polymer nanocapsule.

In some embodiments, an amount of neutral monomer ranges from between about 40% to about 70% by total weight of the polymer shell of the polymer nanocapsule. In other embodiments, an amount of neutral monomer ranges from between about 40% to about 65% by total weight of the total weight of the polymer shell of the polymer nanocapsule. In yet other embodiments, an amount of neutral monomer ranges from between about 45% to about 65% by total weight of the total weight of the polymer shell of the polymer nanocapsule. In further embodiments, an amount of neutral monomer ranges from between about 45% to about 60% by total weight of the total weight of the polymer shell of the polymer nanocapsule.

In some embodiments, an amount of crosslinker (e.g. an erodible or degradable crosslinker) ranges from between about 5% to about 20% by total weight of the polymer shell of the polymer nanocapsule. In other embodiments, an amount of crosslinker ranges from between about 5% to about 15% by total weight of the total weight of the polymer shell of the polymer nanocapsule. In yet other embodiments, an amount of crosslinker ranges from between about 5% to about 12.5% by total weight of the total weight of the polymer shell of the polymer nanocapsule. In other embodiments, an amount of crosslinker ranges from between about 5% to about 10% by total weight of the total weight of the polymer shell of the polymer nanocapsule.

In some embodiments, a ribonucleoprotein or a ribonucleoprotein complex may comprise between about 10% to about 50% by total weight of the polymer nanocapsule including its payload. In other embodiments, a ribonucleoprotein or a ribonucleoprotein complex may comprise between about 15% to about 45% by total weight of the polymer nanocapsule including its payload. In yet other embodiments, a ribonucleoprotein or a ribonucleoprotein complex may comprise between about 20% to about 40% by total weight of the polymer nanocapsule including its payload.

In some embodiments, a ratio of positively charged monomer to neutrally charged monomer ranges from about 6:1 to about 1:6. In other embodiments, the ratio of positively charged monomer to neutrally charged monomer ranges from about 5:1 to about 1:5. In other embodiments, the ratio of positively charged monomer to neutrally charged monomer ranges from about 4:1 to about 1:4. In other embodiments, the ratio of positively charged monomer to neutrally charged monomer ranges from about 3:1 to about 1:3. In yet other embodiments, the ratio of positively charged monomer to neutrally charged monomer ranges from about 1:1 to about 1:5. In yet other embodiments, the ratio of positively charged monomer to neutrally charged monomer ranges from about 1:1 to about 1:4. In yet other embodiments, the ratio of positively charged monomer to neutrally charged monomer ranges from about 1:1 to about 1:3. In yet other embodiments, the ratio of positively charged monomer to neutrally charged monomer ranges from about 1:1 to about 1:2. The skilled artisan will appreciate that as the amount of positively charged monomer relative to the amount of neutral monomer increases, the amount of positive surface charge of the polymer nanocapsule formed therefrom increases (i.e. the resulting polymer nanocapsules have a net positive charge). Likewise, if the amount of neutral monomer increases with respect to the amount of positively charged monomer, then the polymer nanocapsule formed therefrom may have a neutral charge or a slightly negative charge.

In some embodiments, a ratio of monomers (positively charged and neutrally charged monomers) to crosslinkers ranges from about 1:2 to about 1:10. In other embodiments, a ratio of monomers (positively charged and neutrally charged monomers) to crosslinkers ranges from about 1:2 to about 1:9. In other embodiments, a ratio of monomers (positively charged and neutrally charged monomers) to crosslinkers ranges from about 1:2 to about 1:8. In some embodiments, a ratio of monomers (positively charged and neutrally charged) to crosslinkers ranges from about 1:3 to about 1:8.

| Component | Formulation | Formulation | Formulation | Formulation |
|---|---|---|---|---|
| Name | 1 | 2 | 3 | 4 |
| Positive | 10% | 5% | 10% | 20% |
| Monomer 1 | | | | |
| Positive | 30% | 20% | 20% | 30% |
| Monomer 2 | | | | |
| Crosslinker | 10% | 10% | 10% | 5% |
| Neutral | 50% | 65% | 60% | 45% |
| Monomer | | | | |

In some embodiments, and with reference to the Table which follows, the molar ratio of positively (“monomer 1”), neutral hydrophilic monomer (“monomer 2”) and crosslinker to different payloads, such as horseradish peroxidase, siRNA duplex, sh5 DNA cassette, antibody, Cas9 protein, RNP (Cas9 and gRNA complex) and adeno virus, can be estimated based on the molecular weight and net charge of the cargo to be included within the formed nanocapsule.

| | Molecular | | | | Degradable |
|---|---|---|---|---|---|
| | weight | Net | Monomer 1 | Monomer 2 | Crosslinker |
| Payload | (kDa) | Charge | (−1/+1/+2/+3) | (0) | (0) |
| Horseradish peroxidase | 44 | −3 | Condensation | 3 to 10 × | 10 to 30% of |
| siRNA duplex | 13.3 | −42 | factor × Net | Monomer 1 | (monomer 1 + |
| Sh5 DNA cassette | 264 | −800 | charge of | | monomer 2) |
| (400 bps) | | | payload/net | | |
| Antibody | 160 | −10 | charge of | | |
| Cas9 | 160 | 22 | Monomer 1 | | |
| (Streptococcus pyogenes) | | | | | |
| RNP (Cas9 sp + gRNA) | 194 | −81 | | | |
| Adeno type 5 | 157000 | −12000 | | | |

In some embodiments, the overall net charge of the surface of the nanocapsule is positive. For example, in some embodiments, the surface of the nanocapsule can have a charge of between about 1 to about 15 millivolts (mV) (such as measured in a standard phosphate solution). In other embodiments, the surface of the nanocapsule can have a charge of between about 1 to about 10 mV. In yet other embodiments, the surface of the nanocapsule can have a charge of between about 5 to about 10 mV. In further embodiments, the surface of the nanocapsule can have a charge between about 1 to about 5 mV. In even further embodiments, the surface of the nanocapsule can have a charge ranging from between about 3 to about 5 mV. All of the recited charges are as measured using a Zetasizer-NanoZS (available from Malvern Panalytical Ltd). at a pH of about 7.4. The net positive surface charge of the nanocapsules of the present disclosure is believed to be important for obtaining an interaction between the nanocapsule and a biological surface, particularly between the nanocapsule and a cell membrane.

In some embodiments, the polymer nanocapsules have an average diameter of less than or equal to about 200 nanometers (nm), for example between about 1 to 200 nm, or between about 5 to about 200 nm, or between about 10 to about 150 nm, or 15 to 100 nm, or between about 15 to about 150 nm, or between about 20 to about 125 nm, or between about 50 to about 100 nm, or between about 50 to about 75 nm. In other embodiments, the polymer nanocapsules have an average diameter of between about 10 nm to about 20 nm, about 20 to about 25 nm, about 25 nm to about 30 nm, about 30 nm to about 35 nm, about 35 nm to about 40 nm, about 40 nm to about 45 nm, about 45 nm to about 50 nm, about 50 nm to about 55 nm, about 55 nm to about 60 nm, about 60 nm to about 65 nm, about 70 to about 75 nm, about 75 nm to about 80 nm, about 80 nm to about 85 nm, about 85 nm to about 90 nm, about 90 nm to about 95 nm, about 95 nm to about 100 nm, or about 100 nm to about 110 nm. In yet other embodiments the polymer nanocapsules have an average diameter of between about 120 nm to about 130 nm, about 130 nm to about 140 nm, about 140 nm to about 150 nm, about 150 nm to about 160 nm, about 160 nm to about 170 nm, about 170 nm to about 180 nm, 180 nm to about 190 nm, about 190 nm to about 200 nm, about 200 nm to about 210 nm, about 220 nm to about 230 nm, about 230 nm to about 240 nm, about 240 nm to about 250 nm, or larger than about 250 nm in diameter. It is believed that the size of the polymer nanocapsules described herein can be determined based on the polymer crosslinker ratio as described herein.

In some embodiments, the polymer nanocapsules are designed to degrade in about 1 hour, or about 2 hours, or about 3 hours, or about 4 hours, or about 5 hours, or about 6 or about 12 hours, or about 1 day, or about 2 days, or about 1 week, or about 1 month. In other embodiments, the polymer nanocapsules are designed to degrade at any of the rates specified above at a physiological pH. In specific embodiments, the polymer nanocapsules are designed to degrade at any of the rates specified above post-administration to a subject in need thereof.

In some embodiments, the ribonucleoprotein complexes include a Cas protein including one or more the nuclear localization signal fusion peptides (NLSs). In some embodiments, each Cas endonuclease, e.g. a Cas9 endonuclease, includes, 1, 2, 3 or more NLSs. In some embodiments, the presence of the one or more NLSs facilitate nuclear transport of the ribonucleoprotein complexes.

Polymer Nanocapsule Conjugates

Also disclosed herein are conjugates of a polymer nanocapsule (including any payload) and another molecular entity (hereinafter referred to as “conjugates” or “polymer nanocapsule conjugates.”). In some embodiments, the molecular entity conjugated to the polymer nanocapsule comprises a targeting moiety and/or a stabilizing moiety. In some embodiments, the targeting moieties and/or the stabilizing moieties are coupled to the surface of the polymer shell of the polymer nanocapsule. It is believed that the conjugation of the one or more targeting moieties and/or one or more stabilizing moieties to the polymer nanocapsules provides for one or more of (i) specific targeting of the polymer nanocapsules to the surface of a specific cell type thereby providing directed delivery of the ribonucleoprotein complexes to the specific cell type, (ii) enhancement of the water solubility of the conjugate, (iii) enhancement of the stability of the polymer nanocapsules/conjugates against serum protein, and/or (iv) reduction of non-specificity for targeted delivery.

In some embodiments, the polymer nanocapsule conjugates comprise one or more targeting moieties. In other embodiments, the polymer nanocapsule conjugates comprise one or more stabilizing moieties. In yet other embodiments, the polymer nanocapsule conjugates comprise one or more targeting moieties and one or more stabilizing moieties. In some embodiments, the polymer nanocapsule conjugates comprise at least one targeting moiety and at least two stabilizing moieties. In other embodiments the polymer nanocapsule conjugates comprise at least two targeting moieties and at least one stabilizing moiety.

In some embodiments, of the polymer nanocapsule conjugates comprise one or more targeting and/or one or more stabilizing moieties, and wherein a total number of the one or more targeting and/or stabilizing moieties coupled to the polymer nanocapsule ranges from between about 1 and about 24. In other embodiments, of the polymer nanocapsule conjugates comprise one or more targeting and/or one or more stabilizing moieties, and wherein a total number of the one or more targeting and/or stabilizing moieties coupled to the polymer nanocapsule ranges from between about 1 and about 16. In some embodiments, of the polymer nanocapsule conjugates comprise one or more targeting and/or one or more stabilizing moieties, and wherein a total number of the one or more targeting and/or stabilizing moieties coupled to the polymer nanocapsule ranges from between about 1 and about 12. In some embodiments, of the polymer nanocapsule conjugates comprise one or more targeting and/or one or more stabilizing moieties, and wherein a total number of the one or more targeting and/or stabilizing moieties coupled to the polymer nanocapsule ranges from between about 2 and about 12. In some embodiments, of the polymer nanocapsule conjugates comprise one or more targeting and/or one or more stabilizing moieties, and wherein a total number of the one or more targeting and/or stabilizing moieties coupled to the polymer nanocapsule ranges from between about 2 and about 9. In some embodiments, of the polymer nanocapsule conjugates comprise one or more targeting and/or one or more stabilizing moieties, and wherein a total number of the one or more targeting and/or stabilizing moieties coupled to the polymer nanocapsule ranges from between about 2 and about 8. In some embodiments, of the polymer nanocapsule conjugates comprise one or more targeting and/or one or more stabilizing moieties, and wherein a total number of the one or more targeting and/or stabilizing moieties coupled to the polymer nanocapsule ranges from between about 2 and about 6. In some embodiments, of the polymer nanocapsule conjugates comprise one or more targeting and/or one or more stabilizing moieties, and wherein a total number of the one or more targeting and/or stabilizing moieties coupled to the polymer nanocapsule ranges from between about 2 and about 4. In some embodiments, of the polymer nanocapsule conjugates comprise one or more targeting and/or one or more stabilizing moieties, and wherein a total number of the one or more targeting and/or stabilizing moieties coupled to the polymer nanocapsule ranges from between about 3 and about 6. The skilled artisan will appreciate that larger polymer nanocapsules (e.g. those having a greater total surface area) will be able to facilitate the incorporation of more targeting moieties and/or stabilizing moieties as compared with respectively smaller polymer nanocapsules.

In some embodiments, the polymer nanocapsule conjugates have the general structure of Formula (II):

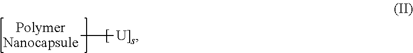

wherein

“Polymer Nanocapsule” is a polymer nanocapsule including a payload (such as any of those polymer nanocapsules and/or payloads described herein);

U is a targeting moiety or stabilizing moiety, and

s is an integer ranging from between 1 and 24.

In some embodiments, s is an integer ranging from between 1 and 16. In some embodiments, s is an integer ranging from between 1 and 12. In some embodiments, s is an integer ranging from between 2 and 12. In some embodiments, s is an integer ranging from between 2 and 9. In some embodiments, s is an integer ranging from between 2 and 8. In some embodiments, s is an integer ranging from between 2 and 6. In some embodiments, s is an integer ranging from between 2 and 4. In some embodiments, s is an integer ranging from between 1 and 12. In some embodiments, s is an integer ranging from between 3 and 9. In some embodiments, s is an integer ranging from between 3 and 6. In some embodiments, the conjugates of Formula (II) include at least one targeting moiety and at least one stabilizing moiety. By way of example only, the polymer nanocapsule conjugates of Formula (II) may include one or more targeting moieties including one or more of an anti-CD4 antibody, an anti-CD8 antibody, and an anti-CD45 antibody, such that the polymer nanocapsule conjugate targets a cell, e.g. a host cell, having a cluster of differentiation marker selected from the group consisting of CD4, CD8, =CD45, and any combination thereof.

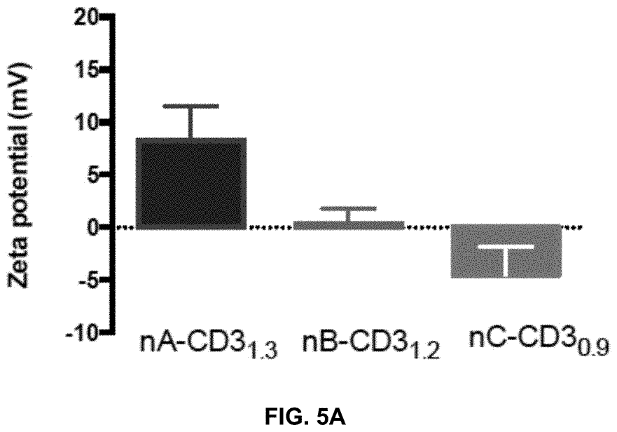

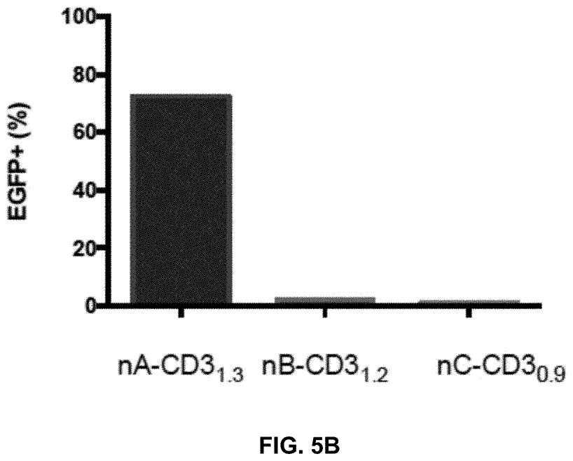

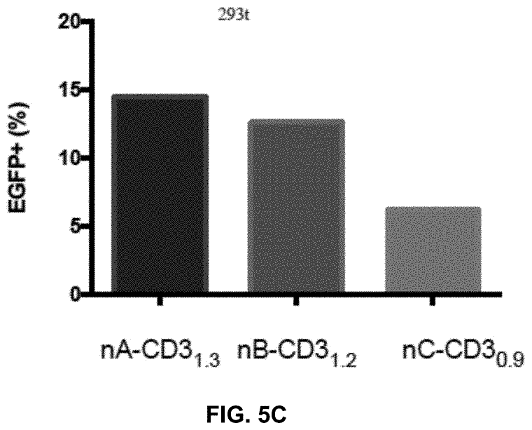

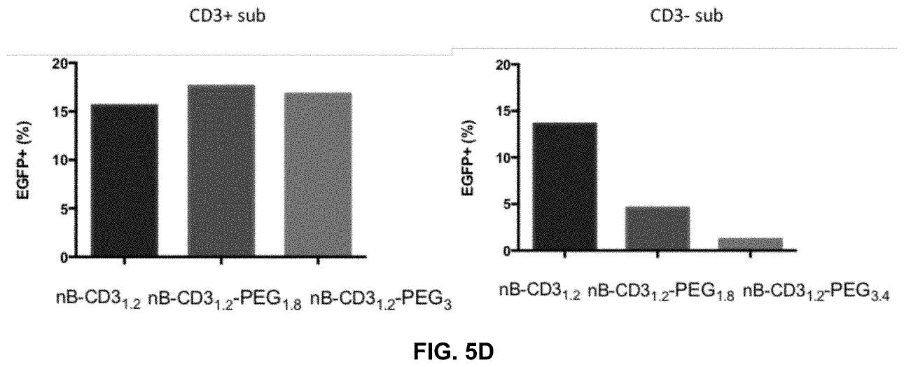

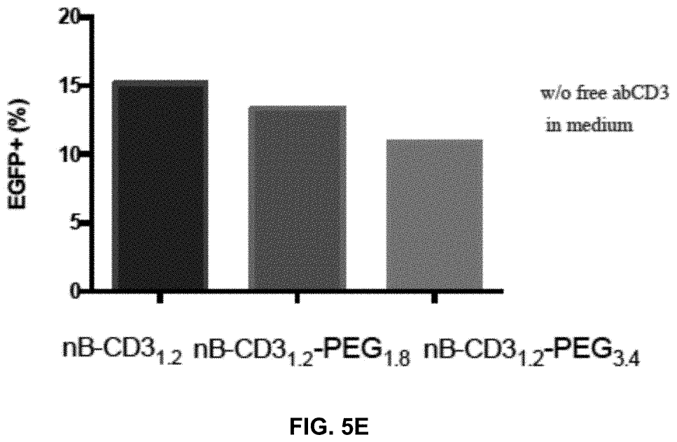

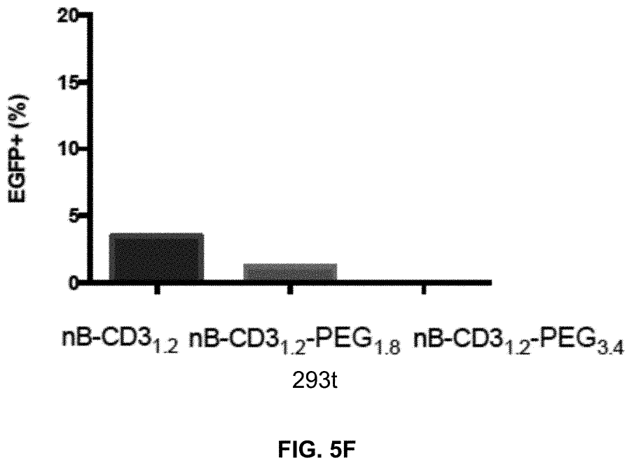

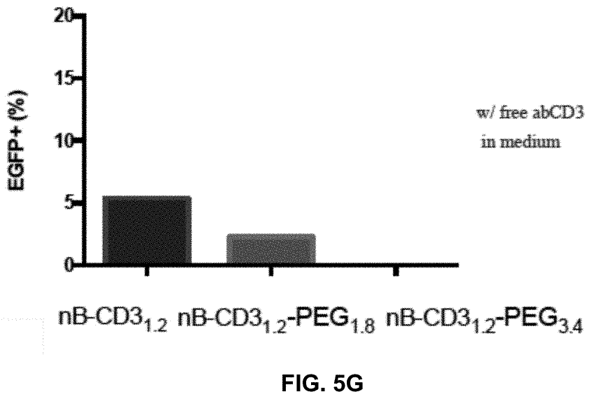

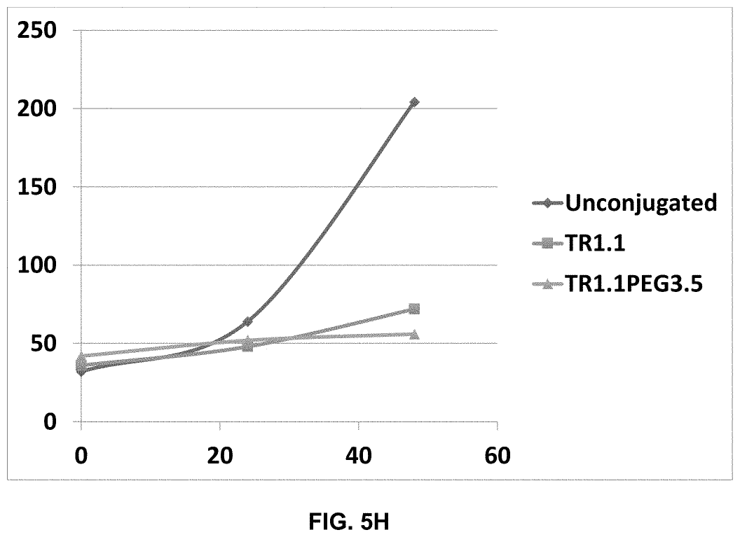

In some embodiments, the polymer nanocapsule conjugates are internalized within a cell via an endocytosis process. In some embodiments, the polymer nanocapsule conjugates do not comprise a targeting moiety (e.g. include one or more stabilizing moieties) and are internalized via a caveolae-mediated endocytosis pathway (see also Yan et. al. “A Novel Intracellular Protein Delivery Platform Based on Single-Protein Nanocapsules, Nature Nano 2010, 5, 48-53, and Yan et. al. “Single siRNA nanocapsules for enhanced RNAi delivery, JACS, 2012, 134, 33, 13542-5, the disclosures of which are hereby incorporated by reference herein in their entireties). In some embodiments, and for nanocapsules not including one or more targeting moieties, the endocytosis process is initiated depending on the charge of the nanocapsule. In some embodiments, a positive charge is required on the polymer nanocapsule to initiate the caveolae-mediated endocytosis pathway. In other embodiments, the polymer nanocapsule conjugates comprise one or more targeting moieties and are internalized via receptor-mediated endocytosis or integrin-mediated endocytosis as described further herein. As demonstrated in FIGS. 5E-5G, endocytosis of the disclosed polymer nanocapsule conjugates is able to occur, even without the presence of imidazolyl groups within the polymer shell of the polymer nanocapsule conjugates.

In some embodiments, it is believed that the endocytosis process proceeds according the following process: the polymer nanocapsule (or polymer nanocapsule conjugate) attaches to the surface of a cell membrane through electrostatic interaction, antibody-receptor interaction, and/or RGD-integrin interaction, where the nanocapsule is then endocytosised, i.e. internalized within the cell within an endosome, namely a membrane-bound compartment. Once within the endosome, protons are pumped into the endosome and the pH within the endosome is lowered, i.e. made more acidic. It is believed that the nanocapsule will then swell and degrade gradually, and the ribonucleoprotein complex will “escape” from the swollen endosome. In some embodiments, it is believed that nuclear localization signal fusion peptides, if present on a Cas protein, will assist nuclear transport of the RNP, i.e. will assist in transporting the RNP payload across the nuclear membrane.

In some embodiments, the polymer nanocapsule conjugates are stable at neutral pHs (e.g. a pH ranging from between 7 and 7.6). In some embodiments, the polymer nanocapsule conjugates degrade at acidic pHs (e.g. a pH ranging from between about 5 to about 6), enabling release of the RNP complex payload to the cytoplasmic.

Tareetine Moieties and Polymer Nanocapsule Conjugates Comprising Targeting Moieties

The present disclosure provides for conjugates comprising one or more targeting moieties and any of the polymer nanocapsules described herein. In some embodiments, conjugation of the one or more targeting moieties to the polymer nanocapsules allows for the payloads of the polymer nanocapsule conjugates, e.g. ribonucleoprotein complexes, to be delivered to specific tissue sites in vivo or to specific cell types either in vivo or ex vivo. In some embodiments, the targeting moiety of the polymer nanocapsule conjugates may enhance accumulation of the polymer nanocapsule in a cell or tissue of interest. For example, the targeting moiety portion of the polymer nanocapsule conjugate may bind to or otherwise associate with a cell-specific cell surface receptor, thereby bringing the polymer nanocapsules into immediate proximity to the target cell.

In some embodiments, the targeting moiety is selected from one which enables delivery of the polymer nanocapsule conjugates to a specific cell type having a specific type of receptor or marker. In some embodiments, the targeting moiety comprises an antibody (e.g. an anti-CD3 antibody, an anti-CD4 antibody, an anti-CD8 antibody, an anti-CD45 antibody), an antibody fragment, a peptide (e.g. a cell penetrating peptide, arginylglycylaspartic acid (RGD), IL4RPep-1), a protein (e.g. lectins, transferrins), a polysaccharide (e.g. hyaluronic acid), carbohydrate, nucleic acids, vitamins (e.g. vitamin D), aptamers (e.g. AS-1411, GBI-10), or small molecules (folate, anisamide, phenylboronic acid), etc. In other embodiments, the targeting moiety comprises cyclodextrin, adamantine, a HLA, or N-Acetylgalactosamine (GalNac). In some embodiments, the targeting moiety comprises an anti-CD34 antibody. In some embodiments, the targeting moiety comprises an anti-CD49f antibody. In some embodiments, the targeting moiety comprises an anti-CD133 antibody. In some embodiments, the targeting moiety comprises an arginine-glycine-aspartic acid (RGD)-containing peptide. In some embodiments, the targeting moiety comprises transferrin or a cyclic variant thereof. In some embodiments, the targeting moiety comprises IL-3.

In some embodiments, the targeting moiety is an antibody and wherein between about 1 and about 5 antibodies are coupled to the polymer nanocapsule. In some embodiments, the targeting moiety is an antibody and wherein between about 1 and about 4 antibodies are coupled to the polymer nanocapsule. In some embodiments, the targeting moiety is an antibody and wherein between about 1 and about 3 antibodies are coupled to the polymer nanocapsule. In some embodiments, the targeting moiety is an antibody and wherein between about 1 and about 2 antibodies are coupled to the polymer nanocapsule.

In some embodiments, the targeting moiety may target a specific cell-surface report specific to immune cells, blood cells, cardiac cells, lung cells, optic cells, liver cells, kidney cells, brain cells, cells of the central nervous system, cells of the peripheral nervous system, cancer cells, cells infected with viruses, stem cells, skin cells, intestinal cells, and/or auditory cells. In some embodiments, the cancer cells are cells selected from the group comprising lymphoma cells, solid tumor cells, leukemia cells, bladder cancer cells, breast cancer cells, colon cancer cells, rectal cancer cells, endometrial cancer cells, kidney cancer cells, lung cancer cells, melanoma cells, pancreatic cancer cells, prostate cancer cells, and thyroid cancer cells. By way of example, a T-cell expressing a CD3 marker may be targeted by an anti-CD3 antibody coupled to a polymer nanocapsule. Following this example, the same polymer nanocapsule coupled to an anti-CD3 antibody would not target a B-cell expressing a CD19 marker.

In some embodiments, the targeting moieties target a cluster of differentiation marker (“CD marker”). In some embodiments, the targeting moieties are anti-CD marker antibodies. The cluster of differentiation (CD) designation refers to cell surface proteins. Each unique molecule is assigned a different number designation, which allows identification of cell phenotypes. Surface expression of a particular CD molecule may not be specific for just one cell or even a cell lineage; however, many are useful for characterization of cell phenotypes. In some embodiments, the targeting moieties target CD markers expressed by stem cells. In some embodiments, the targeting moieties target any one of the CD117 (e.g. an anti-CD117 antibody), CD10 (e.g. an anti-CD10 antibody), CD34 (e.g. an anti-CD34 antibody), CD38 (e.g. an anti-CD38 antibody), CD45 (e.g. an anti-CD45 antibody), CD123 (e.g. an anti-CD123 antibody), CD127 (e.g. an anti-CD127 antibody), CD135 (e.g. an anti-CD135 antibody), CD44 (e.g. an anti-CD44antibody), CD47 (e.g. an anti-CD47 antibody), CD96 (e.g. an anti-CD96antibody), CD2 (e.g. an anti-CD2 antibody), CD4 (e.g. an anti-CD4 antibody), CD3 (e.g. an anti-CD3antibody), and CD9 (e.g. an anti-CD9 antibody), markers. In some embodiments, the targeting moiety targets any one of a human mesenchymal stem cell CD marker, including the CD29 (e.g. an anti-CD29 antibody), CD44 (e.g. an anti-CD44antibody), CD90 (e.g. an anti-CD90 antibody), CD49a-f (e.g. an anti-CD49a-f antibody), CD51 (e.g. an anti-CD117 antibody), CD73 (SH3), CD105 (SH2), CD106 (e.g. an anti-CD106 antibody), CD166 (e.g. an anti-CD166 antibody), and Stro-1 markers. In some embodiments, the targeting moiety targets any one of a human hematopoietic stem cell CD marker including CD34 (e.g. an anti-CD34antibody), CD38 (e.g. an anti-CD38 antibody), CD45RA (e.g. an anti-CD45A antibody), CD90 (e.g. an anti-CD90 antibody), and CD49 (e.g. an anti-CD49 antibody).

In other embodiments, the conjugates used to achieve specific targeting of the polymer nanocapsules include any one or more of AFP, beta-Catenin, BMI-1, BMP-4, c-kit, CXCL12, SDF-1, CXCR4, decorin, E-Cadherin, Cadherin 1, EGFR, ErbB1, Endoglin, EpCAM, TROP-1, Fc epsilon RI A, FCER1A, LiCAM, LMO2, Nodal, Notch-1, PDGFRB, Podoplanin, PTEN, Sonic Hedgehog, STAT3, Syndecan-1, Tranferrin Receptor, and Vimentin.

In other embodiments, the conjugates used to achieve specific targeting of the polymer nanocapsules include any one or more of ALK, AFP, B2M, Beta-hCG, BCR-ABL, BRAF, CA15-3, CA19-9, CA-125, Calcitonin, CEA (Carcinoembryonic antigen), CD20, Chromagranin A, Cytokeratin or fragments thereof, EGFR, Estrogen Receptor, Progesterone Receptor, Fibrin, Fibrinogen, HE4, HER2/neu, IgG variants, KIT, lactate dehydrogenase, Nuclear matrix protein 22, PSA, thyroglobulin, uPA, PAI-1, and Oval. In some embodiments, the targeting moieties may be coupled to the polymer nanocapsules using “click chemistry.” “Click chemistry” is a chemical philosophy, independently defined by the groups of Sharpless and Meldal, that describes chemistry tailored to generate substances quickly and reliably by joining small units together. “Click chemistry” has been applied to a collection of reliable and self-directed organic reactions (Kolb, H. C.; Finn, M. G.; Sharpless, K. B. Angew). Chem. Int. Ed. 2001, 40, 2004-2021). For example, the identification of the copper catalyzed azide-alkyne [3+2] cycloaddition as a highly reliable molecular connection in water (Rostovtsev, V. V.; et al. Angew. Chem. Int. Ed. 2002, 41, 2596-2599) has been used to augment several types of investigations of biomolecular interactions (Wang, Q.; et al. J. Am. Chem. Soc. 2003, 125, 3192-3193; Speers, A. E.; et al. J. Am. Chem. Soc. 2003, 125, 4686-4687; Link, A. J.; Tirrell, D. A. J. Am. Chem. Soc. 2003, 125, 11164-11165; Deiters, A.; et al. J. Am. Chem. Soc. 2003, 125, 11782-11783). In addition, applications to organic synthesis (Lee, L. V.; et al. J. Am. Chem. Soc. 2003, 125, 9588-9589), drug discovery (Kolb, H. C.; Sharpless, K. B. Drug Disc. Today 2003, 8, 1128-1137; Lewis, W. G.; et al. Angew. Chem. Int. Ed. 2002, 41, 1053-1057), and the functionalization of surfaces (Meng, J.-C.; et al. Angew. Chem. Int. Ed. 2004, 43, 1255-1260; Fazio, F.; et al. J. Am. Chem. Soc. 2002, 124, 14397-14402; Collman, J. P.; et al. Langmuir 2004, ASAP, in press; Lummerstorfer, T.; Hoffmann, H. J. Phys. Chem. B 2004, in press) have also appeared.

In some embodiments, following the association of a cell-specific surface receptor with a targeting moiety, the entire nanocapsule, or just the RNP payload, may be internalized through an endocytosis process. In some embodiments, the targeting moiety may bind to a receptor of the cell or tissue of interest, and the targeting moiety may enhance receptor-mediated or integrin-assisted endocytosis of the nanocapsule by the cell or tissue of interest.

In some embodiments, the targeting moiety is coupled to the polymer nanocapsule through a linker and/or spacer that includes at least one cleavable group, such as a group which may be cleaved during an endocytosis process. In some embodiments, the cleavable group is a disulfide group, as described herein. In some embodiments, the cleavable group is a photocleavable group (e.g. DBCO-PHC-NHS, as described herein). In other embodiments, the photocleavable group may be a nitrobenzyl-based group capable of undergoing a Norish Type II reaction. In yet other embodiments, the photocleavable group is a phenacyl group.

A skilled artisan would appreciate that the targeting moiety may be coupled to the polymer nanocapsule by suitable methods in chemical synthesis. For example, in one or more embodiments, it is envisaged that coupling reactions, including homocouplings or cross-couplings, alkylation, condensation, esterification, etherification, or amide formation may be used to couple one or more targeting moieties to the polymer nanocapsules.

In some embodiments, “click chemistry” is utilized to couple one or more targeting moieties to the polymer nanocapsules. Generally, click chemistry encourages reactions that have modular applications that are wide in scope, that have a high chemical yield, that generate inoffensive by-products, that are chemospecific, that require simple reaction conditions, that use readily available starting materials and reagents, that are solvent free or use benign solvents (such as water), that lead to easy product isolation, that have a large thermodynamic driving force to favor a reaction with a single reaction product, and/or that have a high atom economy. While certain of the general criteria can be subjective in nature, and not all criteria need to be met.

The skilled artisan will appreciate that a polymer nanocapsule comprising an appropriate reactive group (e.g. a DBCO group) may form a click adduct with an appropriately functionalized targeting moiety (e.g. one functionalized with an azide group) to undergo the “click” reaction. Indeed, the skilled artisan will recognize that for one member of a pair of click conjugates to react with another member of the pair of click conjugates, and thus form a covalent bond, the two members of the pair of click conjugates must have reactive functional groups capable of reacting with each other. For example, a targeting moiety must comprise a first reactive functional group (e.g. an azide group) capable of participating in a click chemistry reaction with a polymer nanocapsule which has an appropriate second reactive functional group (e.g. a DBCO group). While the aforementioned examples exemplify a reaction between a first member of a pair of click conjugates comprising a DBCO group and a second member of a pair of click conjugates comprising an azide group, the table below sets forth different pairs of reactive functional groups that will react with each other to form a covalent bond by means of “click chemistry.”

| Reactive Functional | Reactive Functional |
|---|---|
| Group on a First | Group on a Second |
| Member of a Pair | Member of a Pair |
| of Click Conjugates | of Click Conjugates |
| DBCO | Azide |
| Alkene | Tetrazine |
| TCO | Tetrazine |
| Maleimide | Thiol |
| DBCO | 1,3-Nitrone |
| Aldehyde or ketone | Hydrazine |
| Aldehyde or ketone | Hydroxylamine |
| Azide | DBCO |
| Tetrazine | TCO |
| Thiol | Maleimide |
| 1,3-Nitrone | DBCO |
| Hydrazine | Aldehyde or ketone |
| Hydroxylamine | Aldehyde or ketone |
| Tetrazine | Alkene |

In some embodiments, a targeting moiety must first be derivatized before it can be conjugated to a polymer nanocapsule. In some embodiments, a targeting moiety (e.g. an antibody having a free reactive group) may be reacted with a compound of Formula (IIIA) to provide a derivatized targeting moiety, i.e. a targeting moiety having a reactive functional group capable of participating in a click chemistry reaction with an appropriately functionalized polymer nanocapsule.

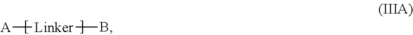

wherein A is maleimide-C(O)—,

“Linker” is a branched or unbranched, linear or cyclic, substituted or unsubstituted, saturated or unsaturated, group having between 2 and 40 carbon atoms, and optionally having one or more heteroatoms selected from O, N, or S, and

B is selected from the group consisting of dibenzocyclooctyne (“DBCO”), trans-cyclooctene (“TCO”), azide, tetrazine, maleimide, thiol, 1,3-nitrone, aldehyde, ketone, hydrazine, and hydroxylamine.

In some embodiments, the moiety A couples to a lysine group or an amine group on a target molecule, such as on a N-terminal group on a protein, such as a Cas9 protein.

In some embodiments, the ‘Linker’ has the structure depicted in Formula (IIIB):

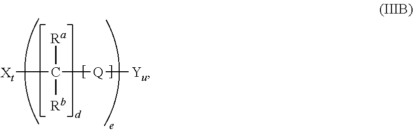

wherein d and e are integers each independently ranging from 2 to 10; t and u are independently 0 or 1; Q is a bond, O, S, or N(R^{c})(R^{d}); R^{a} and R^{b} are independently H, a C₁-C₄ alkyl group, or a halogen; R^{c} and R^{d} are independently CH₃ or H; and X and Y are independently a branched or unbranched, saturated or unsaturated group having between 1 and 4 carbon atoms and optionally having one or more O, N, or S heteroatoms. In some embodiments, X and Y include carbonyl groups, amide groups, ester groups, ester groups, substituted or unsubstituted aryl groups, or any combination thereof. In other embodiments, d and e are integers ranging from 2 to 6.

In some embodiments, the “Linker” has the structure depicted in Formula (IIIC):

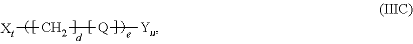

wherein

d and e are integers each independently ranging from 2 to 10;

t and u are independently either 0 or 1;

Q is a bond, O, S, or N(R^{c})(R^{d});

R^{c} and R^{d} are independently CH₃ or H; and

X and Y are independently a branched or unbranched, saturated or unsaturated group having between 1 and 4 carbon atoms and optionally having one or more O, N, or S heteroatoms.

In other embodiments, d and e are integers ranging from 2 to 6.

In some embodiments, the “Linker” has the structure depicted in Formula (HID):

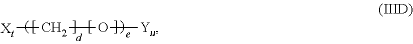

wherein

d and e are integers each independently ranging from 2 to 10;

t and u are independently either 0 or 1; and

X and Y are independently a branched or unbranched, saturated or unsaturated group having between 1 and 4 carbon atoms and optionally having one or more O, N, or S heteroatoms.

In other embodiments, d and e are integers ranging from 2 to 6.

Specific non-limiting examples of compounds of Formula (IIIA) include:

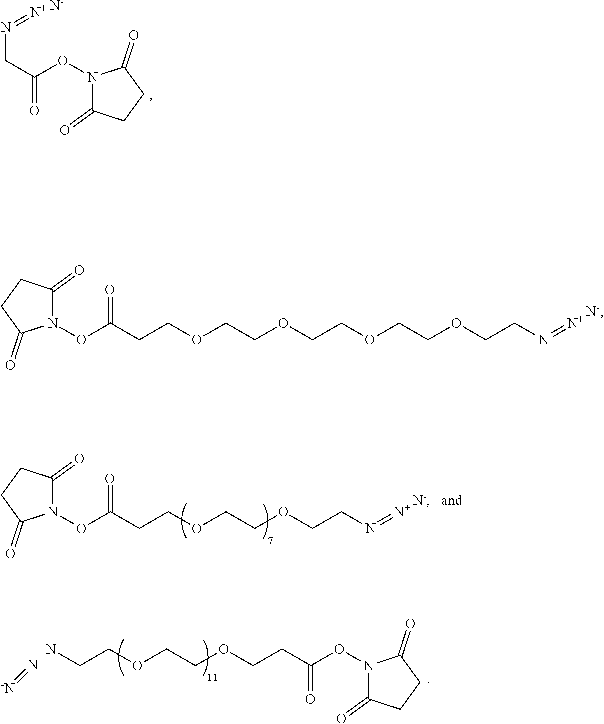

While the above-identified compounds include 4, 8, or 12 polyethylene glycol (“PEG”) groups, respectively, the skilled artisan will appreciate that the compounds of Formula (IIIA) may comprise any number of PEG groups, such as between about 2 and about 20 PEG groups, or between about 2 and about 16 PEG groups, or between about 4 and about 12 PEG groups. The skilled artisan will also appreciate that a polypropylene glycol (“PPG”) group may be substituted for any PEG group, and that the compounds of Formula (IIIA) may comprise any number of PPG groups, such as between about 2 and about 20 PPG groups, or between about 2 and about 16 PPG groups, or between about 4 and about 12 PPG groups.

In embodiments where the targeting moiety is an antibody, functional groups present on an antibody may first be reduced in the presence of dithiothreitol (“DTT”) so as to provide an antibody having one or more thiol groups, and which are reactive the NHS-ester group of the compounds of Formula (IIIA).

The polymer nanocapsules of the present disclosure may likewise be derivatized to include a functional group capable of participating in a click chemistry reaction. In some embodiments, the polymer nanocapsule may be reacted with a compound of Formula (IVA) to provide a polymer nanocapsule having a reactive functional group capable of participating in a click chemistry reaction with an appropriately functionalized targeting moiety. In some embodiments, the polymer nanocapsule includes an amine group which may be reacted with the compounds of Formula (IVA) such that a polymer nanocapsule is functionalized with a functional group capable of participating in a click chemistry reaction.

A-Spacer-B (IVA),

wherein A is maleimide-C(O)—,

“Spacer” is a branched or unbranched, linear or cyclic, substituted or unsubstituted, saturated or unsaturated, group having between 2 and 20 carbon atoms, and having a cleavable group or bond; and

B is selected from the group consisting of dibenzocyclooctyne (“DBCO”), trans-cyclooctene (“TCO”), azide, tetrazine, maleimide, thiol, 1,3-nitrone, aldehyde, ketone, hydrazine, and hydroxylamine.

In some embodiments, the cleavable group comprises a disulfide group.

In some embodiments, the “Spacer” group has the structure of Formula (IVB):

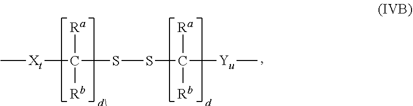

wherein

d is an integer ranging from 2 to 10;

t and u are independently 0 or 1;

R^{a} and R^{b} are independently H, a C₁-C₄ alkyl group, or a halogen; and

X and Y are independently a branched or unbranched, saturated or unsaturated group having between 1 and 8 carbon atoms and optionally having one or more O, N, or S heteroatoms.

In some embodiments, X and Y independently include one or more carbonyl groups, amide groups, ester groups, ester groups, substituted or unsubstituted aryl groups, or any combination thereof. In other embodiments, d and e are integers ranging from 2 to 6.

In some embodiments, the polymer nanocapsules (such as those having between 1 and 10 free amine groups) may be reacted with dibenzocyclooctyne-S—S—N-hydroxysuccinimidyl ester (illustrated below) to provide a polymer nanocapsule bearing a reactive DBCO group, thereby providing a derivatized polymer nanocapsule.

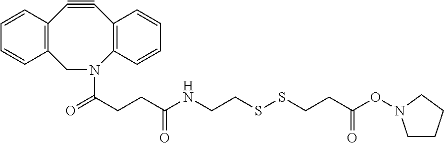 [SMILES: O=C(CCC(=O)N1Cc2ccccc2C#Cc2ccccc21)NCCSSCCC(=O)ON1CCCC1]

In other embodiments, polymer nanocapsules may be reacted with one or more of DBCO-NHS, DBCO-Sulfo-NHS, DBCO-PEG4-NHS, DBCO-PEG5-NHS, DBCO-C6-NHS, or DBCO-PHC-NHS.

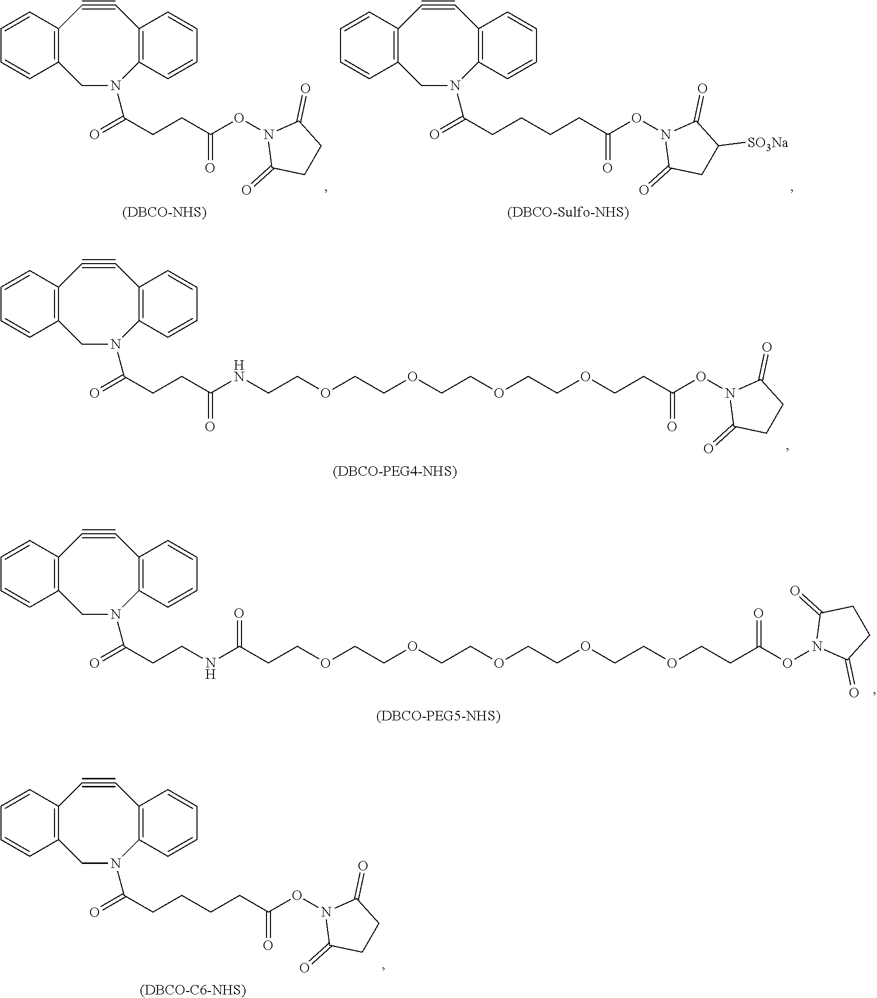

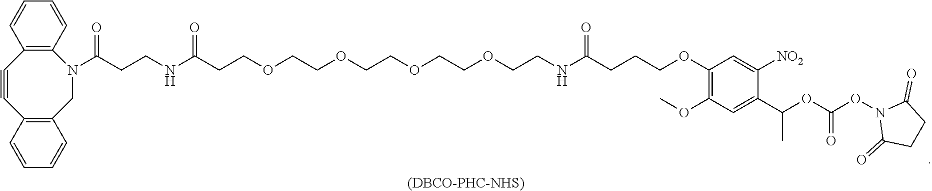 [SMILES: COc1cc(C(C)OC(=O)ON2C(=O)CCC2=O)c([N+](=O)[O-])cc1OCCCC(=O)NCCOCCOCCOCCOCCC(=O)NCCC(=O)N1Cc2ccccc2C#Cc2ccccc21 ; "(DBCO-PHC-NHS)"]

Without wishing to be bound by any particular theory, it is believed that by utilizing a spacer which includes a disulfide bond, the disulfide bond may be cleaved during endocytosis, allowing the polymer nanocapsule, or the contents thereof, to enter the cell without the targeting moiety.

Stabilizine Moieties and Conjugates Comprising Stabilizing Moieties

The present disclosure provides for conjugates comprising one or more stabilizing moieties and any of the polymer nanocapsules described herein. In some embodiments, between about 1 and about 16 stabilizing moieties are coupled to a polymer nanocapsule. In other embodiments, between about 12 and about 12 stabilizing moieties are coupled to a polymer nanocapsule. In yet other embodiments, between about 1 and about 9 stabilizing moieties are coupled to a polymer nanocapsule. In further embodiments, between about 2 and about 8 stabilizing moieties are coupled to a polymer nanocapsule. Of course, the conjugates may further comprise one or more targeting moieties.

A skilled artisan would appreciate that the stabilizing moiety may be coupled to the polymer nanocapsule by suitable methods in chemical synthesis. For example, in one or more embodiments, it is envisaged that coupling reactions, including homocouplings or cross-couplings, alkylation, condensation, esterification, etherification, or amide formation may be used to couple one or more stabilizing moieties to the polymer nanocapsules.

In some embodiments, the stabilizing moieties may be coupled to the polymer nanocapsules using “click chemistry,” such as described above. In some embodiments, and as described above, the polymer nanocapsules may be functionalized with a reactive functional group capable of participating in a click chemistry reaction. For example, and as noted above, the polymer nanocapsules may be functionalized with a DBCO group by reacting the polymer nanocapsule with dibenzocyclooctyne-S—S—N-hydroxysuccinimidyl ester.

A stabilizing moiety bearing a functional group capable of participating in a click chemistry reaction may then be reacted with the functionalized polymer nanocapsule (i.e. a derivatized polymer nanocapsule). In some embodiments, the stabilizing moiety includes one or more polyethylene glycol (PEG) groups and/or polypropylene glycol)PPG) groups. In some embodiments, a PEG-based stabilizing moiety or a PPG-based stabilizing moiety has an average molecular weight ranging from between about 150 Da to about 3000 Da. In some embodiments, a PEG-based stabilizing moiety or a PPG-based stabilizing moiety has an average molecular weight ranging from between about 200 Da to about 2500 Da. In some embodiments, a PEG-based stabilizing moiety or a PPG-based stabilizing moiety has an average molecular weight ranging from between about 250 Da to about 2000 Da. In some embodiments, a PEG-based stabilizing moiety or a PPG-based stabilizing moiety has an average molecular weight ranging from between about 250 Da to about 1500 Da. In some embodiments, a PEG-based stabilizing moiety or a PPG-based stabilizing moiety has an average molecular weight ranging from between about 250 Da to about 1250 Da. In some embodiments, a PEG-based stabilizing moiety or a PPG-based stabilizing moiety has an average molecular weight ranging from between about 250 Da to about 1000 Da. In some embodiments, a PEG-based stabilizing moiety or a PPG-based stabilizing moiety has an average molecular weight ranging from between about 250 Da to about 750 Da. In some embodiments, a PEG-based stabilizing moiety or a PPG-based stabilizing moiety has an average molecular weight ranging from between about 250 Da to about 500 Da.

Other suitable stabilizing moieties include those having Formula (V):

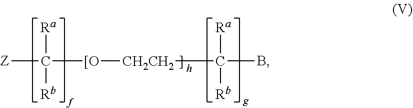

wherein B is selected from the group consisting of dibenzocyclooctyne (“DBCO”), trans-cyclooctene (“TCO”), azide, tetrazine, maleimide, thiol, 1,3-nitrone, aldehyde, ketone, hydrazine, and hydroxylamine;

Z is a hydroxyl group, a branched or unbranched C₁-C₄ alkyl group, —O-alkyl, —NH₂;

f and g are independently 0 or an integer ranging from 1 to 4; and

h is an integer ranging from 1 to 24.

Particular non-limiting examples of stabilizing moieties having Formula (V) include, but are not limited to:
O-(2-Aminoethyl)-O′-(2-azidoethyl)heptaethylene glycol,
O-(2-Aminoethyl)-O′-(2-azidoethyl)nonaethylene glycol,
O-(2-Aminoethyl)-O′-(2-azidoethyl)pentaethylene glycol,
2-[2-(2-Azidoethoxy)ethoxy]ethanol,
O-(2-Azidoethyl)heptaethylene glycol,
O-(2-Azidoethyl)-O′-methyl-triethylene glycol,
O-(2-Azidoethyl)-O′-methyl-undecaethylene glycol,
11-Azido-3,6,9-trioxaundecan-1-amine, and
Methoxypolyethylene glycol azide.

In some embodiments, the stabilizing moieties having Formula (V) comprise (or are derived from) Azido-PEG-amines, where the number of PEG groups ranges from 1 to 24. Suitable non-limiting examples of Azido-PEG-amines include: Azido-PEG1-amine, Azido-PEG2-amine, Azido-PEG3-amine, Azido-PEG4-amine, Azido-PEG5-amine, Azido-PEG6-amine, Azido-PEG7-amine, Azido-PEG8-amine, Azido-PEG10-amine, Azido-PEG11-amine, Azido-PEG20-amine, and Azido-PEG24-amine.

In some embodiments, the stabilizing moieties having Formula (V) comprise (or are derived from) Azido-PEG-alcohols, where the number of PEG groups ranges from 1 to 24. Suitable non-limiting examples of Azido-PEG-alcohols include: Azido-PEG2-alcohol, Azido-PEG3-alcohol, Azido-PEG4-alcohol, Azido-PEG5-alcohol, Azido-PEG6-alcohol, Azido-PEG7-alcohol, Azido-PEG8-alcohol, Azido-PEG9-alcohol, Azido-PEG10-alcohol, Azido-PEG11-alcohol, Azido-PEG12-alcohol, Azido-PEG16-alcohol, Azido-PEG20-alcohol, and Azido-PEG24-alcohol.

In some embodiments, the stabilizing moieties having Formula (V) comprise Azido-PEG-methyl, where the number of PEG groups ranges from 1 to 24. Suitable non-limiting examples of Azido-PEG-methyl include: Azido-PEG1-methyl, Azido-PEG2-methyl, Azido-PEG3-methyl, Azido-PEG4-methyl, Azido-PEG5-methyl, Azido-PEG6-methyl, Azido-PEG7-methyl, Azido-PEG8-methyl, Azido-PEG9-methyl, Azido-PEG10-methyl, Azido-PEG11-methyl, Azido-PEG12-methyl, Azido-PEG16-methyl, Azido-PEG20-methyl, and Azido-PEG24-methyl.

In some embodiments, the stabilizing moieties having Formula (V) comprise Azido-PEG-(CH₂)₃OH, where the number of PEG groups ranges from 1 to 24. Suitable examples of such Azido-PEG-(CH₂)₃OH compounds include Azido-PEG3-(CH₂)₃OH and Azido-PEG4-(CH₂)₃OH.

Method of Delivering a Ribonucleoprotein Complex

Another aspect of the present disclosure is a method of delivering a payload, e.g. a ribonucleoprotein complex, to a cell. In some embodiments, the method comprises contacting any of the polymer nanocapsules or polymer nanocapsule conjugates described herein (including those having ribonucleoprotein-containing core) with a cell, e.g. a host cell. In some embodiments, the method facilitates the delivery of the payload, e.g. a ribonucleoprotein complex, to the nucleus of the cell. In some embodiments, the cell can be a mammalian cell, for example, a human cell. In some embodiments, the cell can be a neuronal cell, a T-cell, a fibroblast, an epithelial cell, a tumor cell, a muscle cell, a skin cell, or an immune system cell.

In some embodiments, the payload, e.g. a ribonucleoprotein complex, is released from the polymer nanocapsule or polymer nanocapsule conjugate upon contacting the polymer nanocapsule with the cell. In some embodiments, the polymer nanocapsule is transported across a cell membrane upon contact with the cell. In some embodiments, the polymer nanocapsule is internalized into a cell through an endocytosis process. In some embodiments, the polymer nanocapsule is internalized through a receptor-mediated endocytosis process. In some embodiments, the polymer nanocapsule includes one or more targeting moieties that bind to surface membrane protein receptors of the cell resulting in endocytosis. In some embodiments, the polymer nanocapsule includes an antibody targeting a specific type of receptor (e.g. a CD marker) and, following the association of the nanocapsule bound targeting antibody with a cellular receptor, the nanocapsule (or its payload) are internalized through an endocytosis process. In some embodiments, some or all of targeting antibodies and/or solubilizing moieties conjugated to the polymer nanocapsule are cleaved during the endocytosis process.

In some embodiments, a ribonucleoprotein complex is released from the polymer nanocapsule after being transported across a cell membrane. In some embodiments, at least 50% of the ribonucleoprotein complex is released from the nanocapsule (based on the total amount of the ribonucleoprotein complex in the polymer nanocapsule). In other embodiments, at least 60% of the ribonucleoprotein complex is released from the nanocapsule (based on the total amount of the ribonucleoprotein complex in the polymer nanocapsule). In yet other embodiments, at least 75% of the ribonucleoprotein complex is released from the nanocapsule (based on the total amount of the ribonucleoprotein complex in the polymer nanocapsule). In further embodiments, at least 90% of the ribonucleoprotein complex is released from the nanocapsule (based on the total amount of the ribonucleoprotein complex in the polymer nanocapsule). In even further embodiments, at least 95% of the ribonucleoprotein complex is released from the nanocapsule (based on the total amount of the ribonucleoprotein complex in the polymer nanocapsule).

In some embodiments, the contacting of the cell with the polymer nanocapsule or the polymer nanocapsule conjugate is performed in vitro. In some embodiments, the contacting of the cell with the polymer nanocapsule or the polymer nanocapsule conjugate is performed in vivo, for example, in the body of a subject or patient, for example, a human (Homo sapiens) or other animal. In some embodiments, the polymer nanocapsule or polymer nanocapsule conjugate is present in the cell in an amount effective to provide a detectable effect in the subject during or after release of the ribonucleoprotein complex, e.g., a therapeutic effect. In some embodiments, the observed or detectable effect arises from cell penetration of the polymer nanocapsule and release of the ribonucleoprotein complex from the nanocapsule. In some embodiments, for in vivo preparation of stem cells, due to low frequency of CD34+ cells in bone marrow (less than about 1 to about 2%), the polymer nanocapsule conjugates including one or more anti-CD34 antibodies are utilized to target CD34+ stem cells. In some embodiments, the CD34+ cells are pre-stimulated with cytokines prior to formulation with the nanocapsules, i.e. a patient is first treatment with cytokines prior to receiving a transfusion of the polymer nanocapsule conjugates.

On the other hand, for ex vivo preparation of stem cells, collected stem cells (e.g. CD34+ stem cells) may, in some embodiments, be pre-stimulated with FLT3-L (Fms-related tyrosine kinase 3 ligand), thrombopoietin (TPO) and/or stem cell factor (SCF), such as overnight. The pre-stimulated stem cells may then be incubated with the polymer nanocapsules or the polymer nanocapsule conjugates of the present disclosure for a time period ranging from between about 3 to about 10 hours, or from about 4 hours to about 8 hours, or from about 4 hours to about 6 hours. After this initial incubation, the stem cells may then be incubated in fresh culture media for between about 24 to about 48 hours prior to cryopreservation. A therapeutically effective amount of the cryopreserved gene-edited stem cells may be transfused to a patient in need of a stem cell treatment.

Methods of Treatment Utilizing Nanocapsules

The nanocapsules and pharmaceutical formulations thereof described herein can be used for the diagnosis, treatment, or prevention of a disease, disorder, syndrome, or a symptom thereof. An amount of the polymer nanocapsules and pharmaceutical formulations thereof described herein can be administered to a subject in need thereof one or more times per day, week, month, or year.

In some embodiments the disease or disorder is a monogenic disease or disorder. Non-limiting examples of monogenic diseases or disorders include Thalassaemia, Sickle cell anemia, Haemophilia, Cystic Fibrosis, Tay sachs disease, Fragile X syndrome and Huntington's disease.

In some embodiments, the amount administered can be the effective amount of the polymer nanocapsules or pharmaceutical formulations thereof. For example, the nanocapsules or pharmaceutical formulations thereof, can be administered in a daily dose. This amount may be given in a single dose per day. In other embodiments, the daily dose may be administered over multiple doses per day, in which each containing a fraction of the total daily dose to be administered (sub-doses). In some embodiments, the number of doses delivered per day is 2, 3, 4, 5, or 6. In further embodiments, the compounds, formulations, or salts thereof are administered one or more times per week, such as 1, 2, 3, 4, 5, or 6 times per week. In other embodiments, the nanocapsules or pharmaceutical formulations thereof are administered one or more times per month, such as 1 to 5 times per month. In still further embodiments, the nanocapsules or pharmaceutical formulations thereof are administered one or more times per year, such as 1 to 11 times per year.

In embodiments where more than one of the polymer nanocapsules, pharmaceutical formulations thereof, and/or auxiliary agent(s) are administered sequentially; the sequential administration may be close in time or remote in time. For example, administration of the second nanocapsules, pharmaceutical formulations thereof, and/or auxiliary agent(s) can occur within seconds or minutes (up to about 1 hour) after administration of the first agent (close in time). In other embodiments, administration of the second nanocapsules, pharmaceutical formulations thereof, and/or auxiliary agent(s) occurs at some other time that is more than an hour after administration of the first the nanocapsules or pharmaceutical formulations thereof.

The amount of the polymer nanocapsules, pharmaceutical formulations thereof, and/or auxiliary agent(s) described herein can be administered in an amount ranging from about 0.01 mg to about 1 g per day, as calculated as the free or unsalted pharmaceutical formulations. The amount of nanocapsules, pharmaceutical formulations thereof, and/or auxiliary agent(s) described herein can be administered in an amount ranging from about 0.01 μM to about 100 μM per day.

In some embodiments, the polymer nanocapsules are used to facilitate delivery of genome editing molecules. In further embodiments, a cell or population of cells can be incubated for period of time with one or more polymer nanocapsules or formulations thereof described herein. In some embodiment, the nanocapsule contains a Cas9:sgRNA complex. The incubation time can range from about 1 h to about 10 days or more. Subsequent to incubation, the cell or cells can be cultured using techniques known in the art and/or described herein. The cell or cells can also be analyzed for genome modification using techniques and/or methods known in the art or as described herein.

Polymer Nanocapsule Formulations

The polymer nanocapsules described herein can be provided to a subject alone or contacted with a cell (in vivo or in vitro) or as an ingredient, such as an active ingredient, in a pharmaceutical formulation or other composition. As such, also described herein are pharmaceutical formulations containing one or more of the nanocapsules described herein. In some embodiments, the pharmaceutical formulations contain an effective amount of polymer nanocapsules described herein. The pharmaceutical formulations can be administered to a subject in need thereof.

The pharmaceutical formulation may include a homogenous population of polymer nanocapsules. In these embodiments, all of the polymer nanocapsules contained in the pharmaceutical formulation are the same. In other embodiments, the pharmaceutical formulation can contain a heterogeneous population of polymer nanocapsules. In these embodiments, the population of polymer nanocapsules contains at least two polymer nanocapsules that are different from one another, e.g. each population including a different ribonucleoprotein complex, such as different ribonucleoprotein complexes including different gRNAs. The two different polymer nanocapsules can vary from one another in the type of targeting moiety, the quantities of coupled targeting moieties, the type and/or quantity of stabilizing molecules, of and/or the components (or ratio of components) constituting the polymer nanocapsule shell.

Another aspect of the present disclosure includes a composition comprising a plurality of nanocapsules. As used herein, “plurality of nanocapsules” refers to a composition comprising more than one nanocapsule, for example more than 10 nanocapsules. The polymer nanocapsules can include polymer nanocapsules having the above-described structure and components. In some embodiments, the composition comprises the plurality of nanocapsules dispersed in an aqueous solution.

The aqueous solution can comprise water, deionized water, a buffer (e.g., phosphate buffered saline, phosphate buffer, and the like), and the like, or a combination thereof. In some embodiments, the composition comprises 1 to 50 volume percent (vol. %) of the nanocapsules, for example 1 to 20 vol. %, for example 5 to 15 vol. %, based on the total volume of the composition. Accordingly, in some embodiments, the composition comprises 50 to 99 vol. %, or 80 to 99 vol. %, or 85 to 95 vol. % of the aqueous solution, based on the total volume of the composition.

Pharmaceutically Acceptable Carriers and Auxiliary Ingredients and Agents

The pharmaceutical formulations containing an effective amount of the polymer nanocapsules described herein can further include a pharmaceutically acceptable carrier. Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxyl methylcellulose, and polyvinyl pyrrolidone, which do not deleteriously react with the active composition.

The pharmaceutical formulations can be sterilized, and if desired, mixed with auxiliary agents, such as lubricants, preservatives, stabilizers, wetting agents, emulsifiers, salts for influencing osmotic pressure, buffers, coloring, flavoring and/or aromatic substances, and the like which do not deleteriously react with the active composition. In addition to the effective amount of the nanocapsules, the pharmaceutical formulation can also include an effective amount of auxiliary active agents, including but not limited to, DNA, RNA, amino acids, peptides, polypeptides, antibodies, aptamers, ribozymes, guide polynucleotides for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, and chemotherapeutics. Suitable compounds for the auxiliary active agents have been previously described herein in relation to the nanocapsules.

Effective Amounts of the Nanocapsules and Auxiliary Agents

The pharmaceutical formulations can contain an effective amount of the polymer nanocapsules and/or an effective amount of an auxiliary agent. In some embodiments, the effective amount of polymer nanocapsules ranges from between about 0.001 pg to about 10,000 μg. In some embodiments, the effective amount of polymer nanocapsules ranges from between about 0.1 pg to about 5,000 μg. In some embodiments, the effective amount of polymer nanocapsules ranges from between about 1 pg to about 1,000 μg. In some embodiments, the effective amount of polymer nanocapsules ranges from between about 1 pg to about 500 μg. In some embodiments, the effective amount of polymer nanocapsules ranges from between about 100 pg to about 500 μg.

In embodiments where there is an auxiliary active agent contained in the pharmaceutical formulation in addition to the polymer nanocapsules, the effective amount of the auxiliary active agent will vary depending on the auxiliary active agent. In some embodiments, the effective amount of the auxiliary active agent ranges from 0.001 micrograms to about 1 milligram. In other embodiments, the effective amount of the auxiliary active agent ranges from about 0.01 IU to about 1000 IU. In further embodiments, the effective amount of the auxiliary active agent ranges from 0.001 mL to about 1 mL. In yet other embodiments, the effective amount of the auxiliary active agent ranges from about 1% w/w to about 50% w/w of the total pharmaceutical formulation. In other embodiments, the effective amount of the auxiliary active agent ranges from about 1% v/v to about 50% v/v of the total pharmaceutical formulation. In still other embodiments, the effective amount of the auxiliary active agent ranges from about 1% w/v to about 50% w/v of the total pharmaceutical formulation.

Dosage Forms

In some embodiments, the pharmaceutical formulations (such as those including any of the polymer nanocapsules and/or polymer nanocapsule conjugates) described herein may be provided in a dosage form suitable for administration. In some embodiments, the dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, and intradermal. Such formulations may be prepared by any method known in the art.

Dosage forms adapted for oral administration can discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. In some embodiments, this is a subject having cancer. In some embodiments, the cancer is folate positive cancer.

Where appropriate, the dosage forms described herein can be microencapsulated. The dosage form can also be prepared to prolong or sustain the release of any ingredient. In some embodiments, the nanocapsule can be the ingredient whose release is delayed. In other embodiments, the release of an auxiliary ingredient is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, the nanocapsule, auxiliary active ingredient, and/or pharmaceutically acceptable salt thereof can be formulated with a paraffinic or water-miscible ointment base. In other embodiments, the active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, the nanocapsules, derivative thereof, auxiliary active ingredient, and/or pharmaceutically acceptable salt thereof in a dosage form adapted for inhalation is in a particle-size-reduced form that is obtained or obtainable by micronization. In some embodiments, the particle size of the size reduced (e.g. micronized) compound or salt or solvate thereof, is defined by a D50 value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active ingredient, which may be generated by various types of metered dose pressurized aerosols, nebulizers, or insufflators.

In some embodiments, the dosage forms are aerosol formulations suitable for administration by inhalation. In some of these embodiments, the aerosol formulation contains a solution or fine suspension of a nanocapsules, auxiliary active ingredient, and/or pharmaceutically acceptable salt thereof a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal, or an aerosol dispenser fitted with a metering valve (e.g. metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a nanocapsule, derivative thereof, auxiliary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once, once daily, or several times daily, for example 2, 3, 4, or 8 times daily, in which 1, 2, or 3 doses are delivered each time.

For some dosage forms suitable and/or adapted for inhaled administration, the pharmaceutical formulation is a dry powder inhalable formulation. In addition to the nanocapsule, auxiliary active ingredient, and/or pharmaceutically acceptable salt thereof, such a dosage form can contain a powder base such as lactose, glucose, trehalose, mannitol, and/or starch. In some of these embodiments, the conjugate compound, derivative thereof, auxiliary active ingredient, and/or pharmaceutically acceptable salt thereof is in a particle-size reduced form. In further embodiments, a performance modifier, such as L-leucine or another amino acid, cellulose octaacetate, and/or metals salts of stearic acid, such as magnesium or calcium stearate.

In some embodiments, the aerosol formulations are arranged so that each metered dose of aerosol contains a predetermined amount of an active ingredient, such as the one or more of the compounds described herein.

Dosage forms adapted for vaginal administration can be presented as pessaries, tampons, creams, gels, pastes, foams, or spray formulations. Dosage forms adapted for rectal administration include suppositories or enemas.

Dosage forms adapted for parenteral administration and/or adapted for injection (i.v., s.q., i.c.v., i.m. etc.), can include aqueous and/or non-aqueous sterile injection solutions, which can contain anti-oxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and resuspended in a sterile carrier to reconstitute the dose prior to administration. Extemporaneous injection solutions and suspensions can be prepared in some embodiments, from sterile powders, granules, and tablets.

For some embodiments, the dosage form contains a predetermined amount of a nanocapsule provided herein per unit dose. In an embodiment, the predetermined amount of the nanocapsule provided herein is an effective amount of the nanocapsules to diagnose, treat, prevent, or mitigate the symptoms of cancer. In other embodiments, the predetermined amount of the nanocapsules is an appropriate fraction of the effective amount of the active ingredient. Such unit doses may therefore be administered once or more than once a day. Such pharmaceutical formulations may be prepared by any of the methods well known in the art.

The auxiliary active agent can be included in the pharmaceutical formulation or can exist as a stand-alone compound or pharmaceutical formulation that is administered contemporaneously or sequentially with the nanocapsule, derivative thereof or pharmaceutical formulation thereof provided herein. In embodiments where the auxiliary active agent is a stand-alone compound or pharmaceutical formulation, the effective amount of the auxiliary active agent can vary depending on the auxiliary active agent used. In some of these embodiments, the effective amount of the auxiliary active agent ranges from 0.001 micrograms to about 1000 grams. In other embodiments, the effective amount of the auxiliary active agent ranges from about 0.01 IU to about 1000 IU. In further embodiments, the effective amount of the auxiliary active agent ranges from 0.001 mL to about 1 mL. In yet other embodiments, the effective amount of the auxiliary active agent ranges from about 1% w/w to about 50% w/w of the total auxiliary active agent pharmaceutical formulation. In other embodiments, the effective amount of the auxiliary active agent ranges from about 1% v/v to about 50% v/v of the total pharmaceutical formulation. In still other embodiments, the effective amount of the auxiliary active agent ranges from about 1% w/v to about 50% w/v of the total auxiliary agent pharmaceutical formulation.

### EXAMPLES

### Example 1

Cas9 protein stock (67 uM) and gRNA stock (100 uM) were added to 50 mM pH=6.4 50 mM HEPES buffer and 150 uM NaCl to a final concentration of 1 uM. The RNP complex formed from the Cas9 and gRNA was negatively charged. Different polymer nanocapsules were formulated, each different polymer nanocapsule incorporating a gRNA having any of SEQ ID NOS: 8-15.

Then, a chemical cocktail of (i) N-(3-((4-((3-aminopropyl)amino)butyl)amino)propyl)acrylamide, (ii) 2-(dimethylamino)ethyl acrylate, (iii) 1,3-glycerol dimethacrylate, and (iv) acrylamide was dissolved in deoxygenated rnase-free water and is added to the Cas9-gRNA complex tube. The ratio of the various monomers was 250:500:1000:4000 (monomers (i):(ii):(iii):(iv)). Radical polymerization was carried out by adding 0.02 mg of ammonium persulfate dissolved in 2 μL of deoxygenated and deionized water and 0.4 μL of N,N,N′,N′-tetramethylethylenediamine. The reaction was allowed to proceed at 4° C. for 180 min in a nitrogen atmosphere. After completion of polymerization, dialysis or ultrafiltration was used to remove rid excess monomers.

Then, the nanocapsules were purified and conjugated with targeting moieties and PEG. Specifically, an about 30 to about 50 fold excess of formed nanocapsule to dibenzocyclooctyne-S—S—N-hydroxysuccinimidyl ester was used to modify the surface of nanocapsules to reach a final conjugation of between about 3 to about 10 esters per nanocapsule. Then, an about 5 to about 10 fold excess of azido functionalized PEGs having a molecular weight of about 2000 daltons, and an about 5 to about 10 fold excess of azido functionalized abCD3 or abCD34 was used to introduce targeting moieties and stabilization moieties to the surface of the formed polymer nanocapsules. Next, the conjugated nanocapsules were separated from unreacted targeting molecules and PEG and the final product enriched using size-exclusion column chromatography. The generated polymer nanocapsule conjugates had a neutral charge and had a size ranging from between about 50 nm to about 100 nm.

### Example 2—HIV as a Disease Target

Nanocapsules are formulated to contain a DNA molecule encoding sh5 or C46 or formulated to contain and CRISPR/Cas9 ribonucleoprotein complex (RNP). Nanocapsules are also conjugated with anti-CD3, anti-CD4, or anti-CD34 antibodies to target T lymphocytes or CD34+ stem/progenitor cells. They are then injected into the bone marrow of humanized mice at a number of specific locations; e.g. the endosteal niche (intraosseous introduction). The consequent construct containing cells are stimulated to replicate and differentiate by one or a combination of: 1) construct response growth/differentiation agent, 2) specific cytokine and antibodies, 3) alpha9beta1/alpha4beta1 inhibitors. Peripheral blood is examined and then the mice are sacrificed to determine the degree of construct expression, including in the case of sh5/C46 introduction CCR5 down-regulation, C46 expression and in the case of CRISPR/CAS, knock-out of CCR5 and knock-in of C46.

### Example 3—Globinopathies as a Disease Target

Nanocapsules are formulated to contain CRISPR/CAS targeting the BCL11A erythroid enhancer. These formulated nanocapsules are introduced into the bone marrow of humanized mice at a number of specific locations; e.g. the endosteal niche. This will act to knock out the activity of the BCL11A erythroid enhancer which will inhibit BCL11A activity resulting in activation of fetal hemoglobin to rescue sickle cell disease or beta-thalassemia.

### Example 4—HPRT as a Target

It is believed that HPRT-deficient cells (i.e. those that are sensitive to a purine analog, e.g. 6TG, such as those having 20% or less residual HPRT gene expression) can be negatively selected by using a dihydrofolate reductase inhibitor to inhibit the enzyme dihydrofolate reductase (DHFR) in the purine de novo synthetic pathway. This has been developed as a safety procedure to eliminate gene-modified HSCs in case of unexpected adverse effects observed. Should any adverse side effects arise, a patient may be treated with methotrexate (“MTX”) or mycophenolic acid (“MPA”). Adverse side effects include, for example, aberrant blood counts/clonal expansion indicating insertional mutagenesis in a particular clone of cells or cytokine storm.

It is believed that MTX or MPA competitively inhibits dihydrofolate reductase (DHFR), an enzyme that participates in tetrahydrofolate (THF) synthesis. DHFR catalyzes the conversion of dihydrofolate to active tetrahydrofolate. Folic acid is needed for the de novo synthesis of the nucleoside thymidine, required for DNA synthesis. Also, folate is essential for purine and pyrimidine base biosynthesis, so synthesis will be inhibited. MTX or MPA, therefore inhibits the synthesis of DNA, RNA, thymidylates, and proteins. MTX or MPA blocks the de novo pathway by inhibiting DHFR. In HPRT−/− cell, there is no salvage or de novo pathway functional, leading to no purine synthesis, and therefore the cells die. However, the HPRT wild type cells have a functional salvage pathway, their purine synthesis takes place and the cells survive. In some embodiments, an analog or derivative of MTX or MPA may be substituted for MTX or MPA. Derivatives of MTX are described in U.S. Pat. No. 5,958,928 and in PCT Publication No. WO/2007/098089, the disclosures of which are hereby incorporated by reference herein in their entireties.

Given the sensitivity of the modified HSCs produced according to the present disclosure, MTX or MPA may be used to selectively eliminate HPRT-deficient cells. In some embodiments, the MTX or MPA is administered as a single dose. In some embodiments, multiple doses of the MTX or MPA are administered.

Nanocapsules are formulated to contain CRISPR/CAS targeting the HPRT locus. These formulated nanocapsules are introduced into human primary CD4+ or CD8+ T cells. This will act to knock out the activity of HPRT which will inhibit HPRT activity resulting in resistance to 6-thioguanine (6-TG) and sensitive to methotrexate (MTX). Those 6-TG-resistent T cells could provide temporary immuno-support for leukemia patient receiving 6-TG chemotherapy. Those MTX-sensitive T cells can be eliminated with infusion of MTX after patient finishes 6-TG chemotherapy.

### Example 5—HPRT Knockdown Versus Knockout with 6TG Selection

6TG is a purine analog having both anticancer and immune-suppressive activities. Thioguanine competes with hypoxanthine and guanine for the enzyme hypoxanthine-guanine phosphoribosyltransferase (HGPRTase) and is itself converted to 6-thioguanylic acid (TGMP). This nucleotide reaches high intracellular concentrations at therapeutic doses. TGMP interferes several points with the synthesis of guanine nucleotides. It inhibits de novo purine biosynthesis by pseudo-feedback inhibition of glutamine-5-phosphoribosylpyrophosphateamidotransferase—the first enzyme unique to the de novo pathway for purine ribonucleotide. TGMP also inhibits the conversion of inosinic acid (IMP) to xanthylic acid (XMP) by competition for the enzyme IMP dehydrogenase. At one-time TGMP was felt to be a significant inhibitor of ATP:GMP phosphotransferase (guanylate kinase), but recent results have shown this not to be so. Thioguanylic acid is further converted to the di- and tri-phosphates, thioguanosine diphosphate (TGDP) and thioguanosine triphosphate (TGTP) (as well as their 2′-deoxyribosyl analogues) by the same enzymes which metabolize guanine nucleotides.

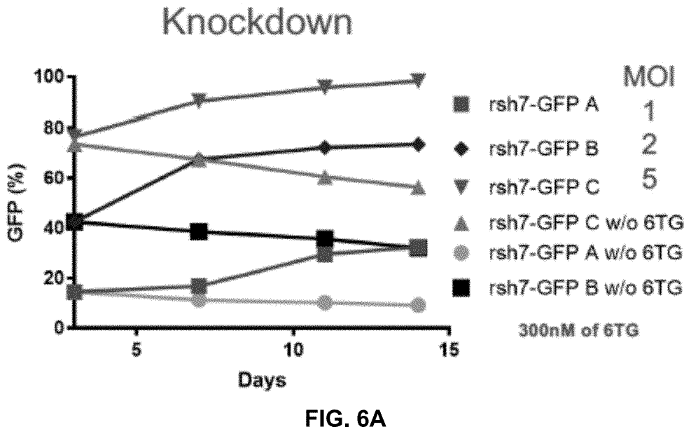

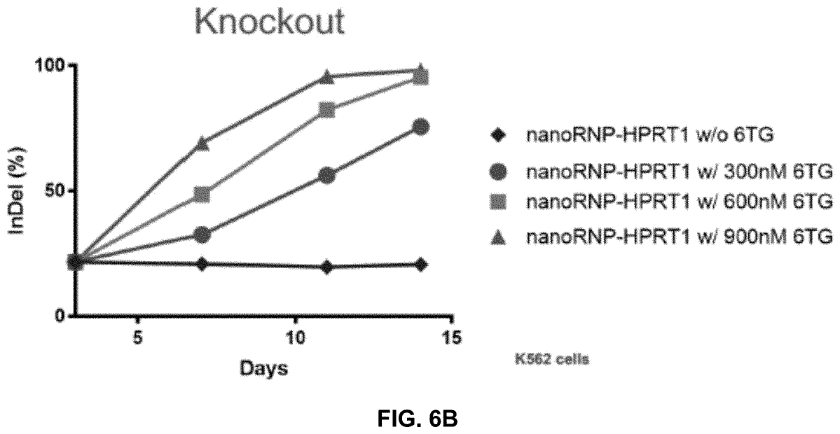

K562 cells were transduced with a vector including a nucleic acid sequence designed to knockdown HPRT and a nucleic acid sequence encoding the green fluorescent protein (GFP)(MOI=1/2/5); or were transfected with a nanocapsule including CRISPR/Cas9 and a sgRNA to HPRT (100 ng/5×10⁴ cells) at day zero (0). 6-TG was added into the medium from day 3 through day 14. The medium was refreshed every 3 to 4 days. GFP was analyzed on flow machine and InDel % as analyzed with T7E1 assay (“Indel” is a molecular biology term for an insertion or deletion of bases in the genome of an organism). FIG. 6A illustrates that the GFP+ population of transduced K562 cells increased from day 3 to day 14 under treatment of 6TG; while the GFP+ population was almost steady without 6-TG treatment. FIG. 6B illustrates that HPRT knockout population of K562 cells increased from day 3 to 14 under treatment of 6TG and higher dosages (900 nM) of 6TG led to faster selection as compared with a dosage of 300/600 nM of 6TG. It should be noted that 6TG selection process occurred much faster on HPRT knockout cells as compared with the HPRT knockdown cells (MOI=1) at the same concentration of 300 nM of 6TG from day 3 to day 14. The difference between knockdown and knockout could be explained by some level of residual HPRT by the RNAi knockdown approach as compared with the full elimination of HPRT by the knockout approach. Therefore, HPRT-knockout cells, in accordance with the present disclosure, were believed to have a much higher tolerance against 6TG and are believed to grow much faster at higher dosages of 6TG (900 nM) compared with HPRT-knockdown cells.

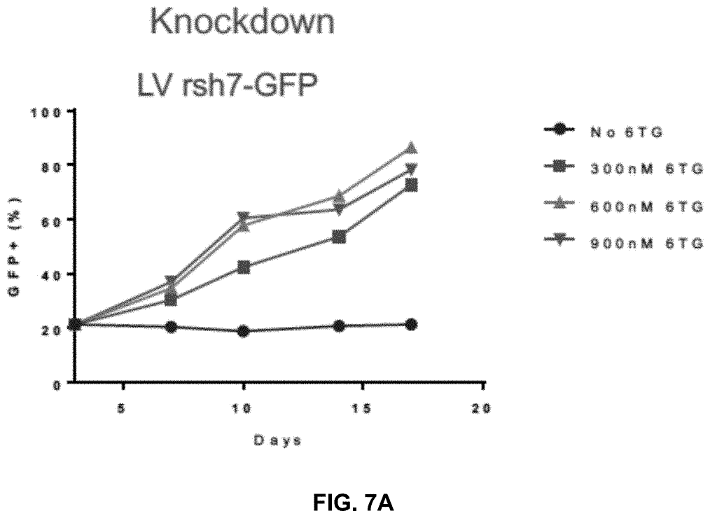

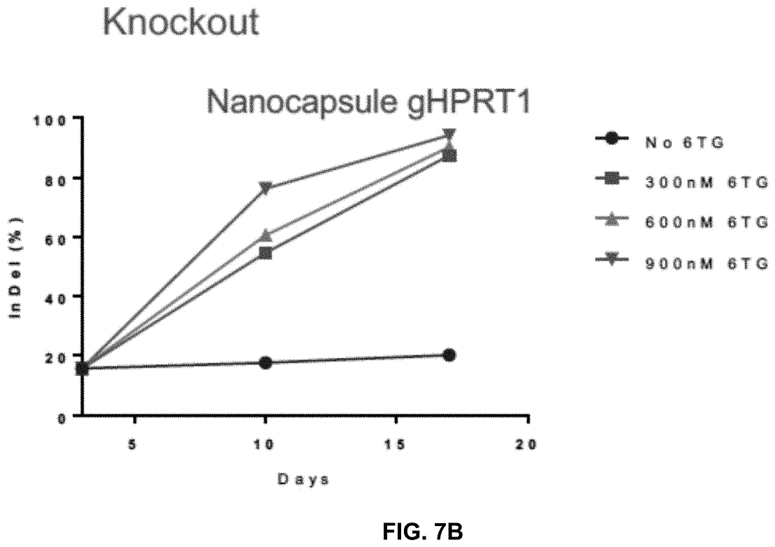

CEM cells were transduced with a vector including a nucleic acid sequence designed to knockdown HPRT and a nucleic acid sequence encoding the green fluorescent protein or transfected with a nanocapsule including CRISPR/Cas9 and a sgRNA to HPRT at day 0. 6-TG was added into the medium from day 3 to day 17. The medium was refreshed every 3 to 4 days. GFP as analyzed on flow machine and InDel % is analyzed by T7E assay. FIG. 7A illustrates that the GFP+ population of transduced K562 cells increased from day 3 to day 17 under treatment of 6TG while GFP+ population was almost steady without 6-TG. FIG. 7B comparatively shows that HPRT knockout population of CEM cells increased from day 3 to 17 under treatment of 6TG and that a higher dosage (900 nM) of 6TG leads to a faster selection as compared with a dosage of 300/600 nM of 6TG. It should be noted that 6TG selection process occurred faster on HPRT knockout cells rather than HPRT knockdown cells (MOI=1) at the same concentration of 6TG from day 3 to day 17.

### Example 6—Knock-Out of HPRT and 6-TG Selection of PBMC With CD3-Conjugated CRISPR Nanocapsules

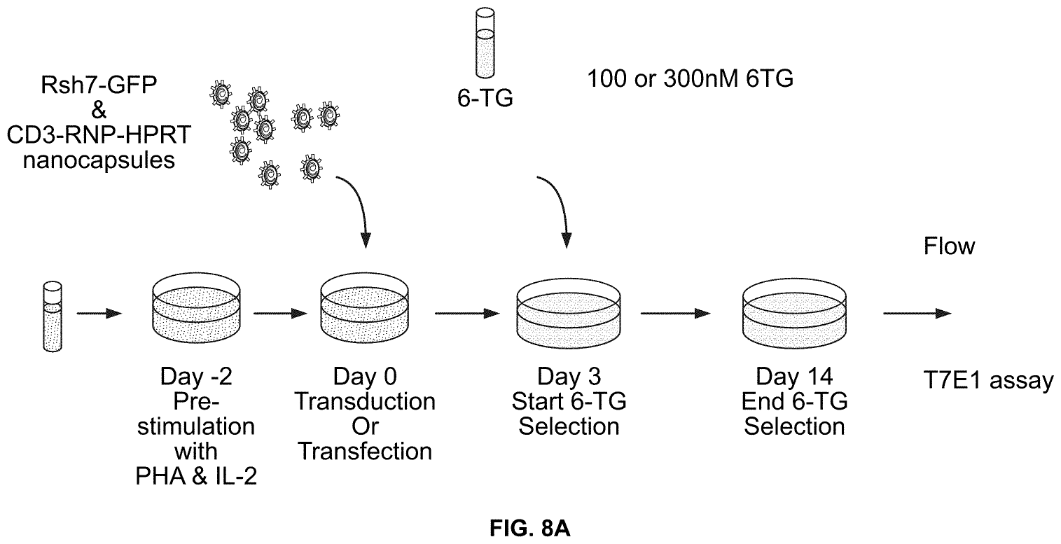

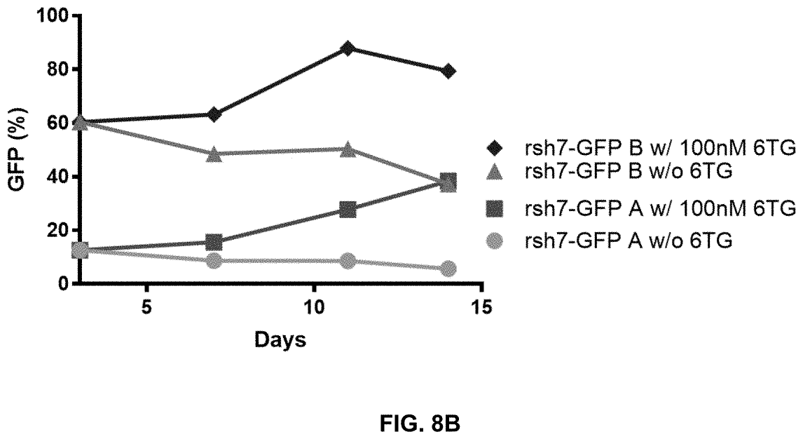

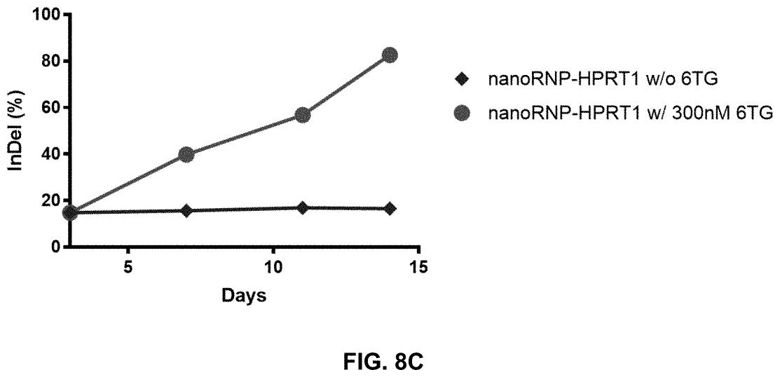

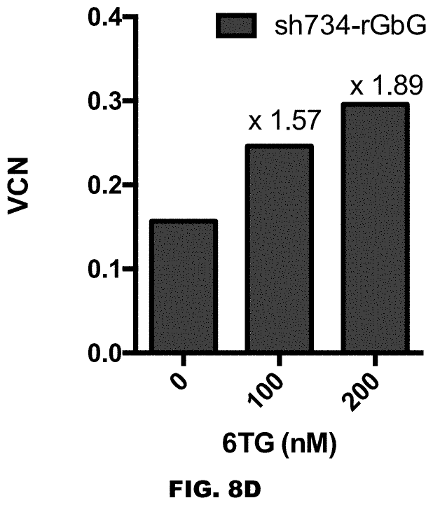

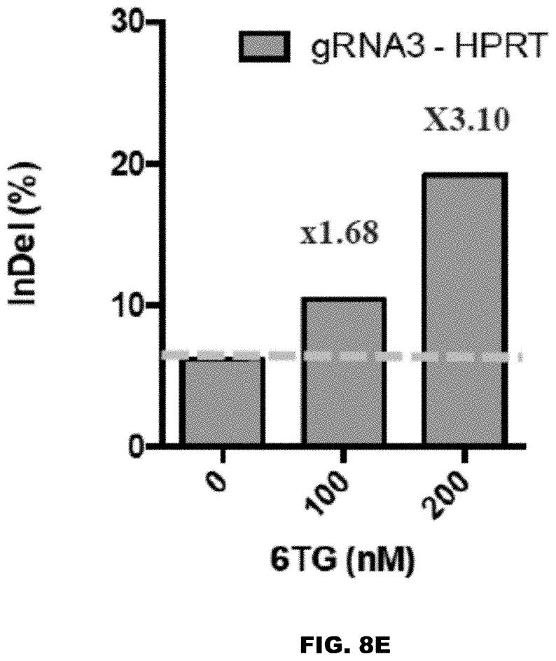

PBMC cells were stimulated with PHA/IL2 2 days before transduction (see FIG. 8A). Stimulated PBMC cells were transduced with LV rsh7-GFP (MOI=0.5) or transfected with nanoRNP-HPRT1 at day 0 (see FIG. 8A). 6-TG was added into the medium from Day 3 to Day 14 (see FIG. 8A). Medium was refreshed every 3-4 days. GFP was analyzed on a flow machine. FIG. 8B illustrates the GFP+ population of Rsh7-GFP transduced PBMC at two different MOIs (2 and 10), where both MOIs increase at 100 nM of 6TG. FIG. 8C illustrated that the HPRT-Knock-out population of PBMCs increased from day 3 to day 14 at 300 nM of 6TG.

### Example 7—Negative Selection with MTX or MPA

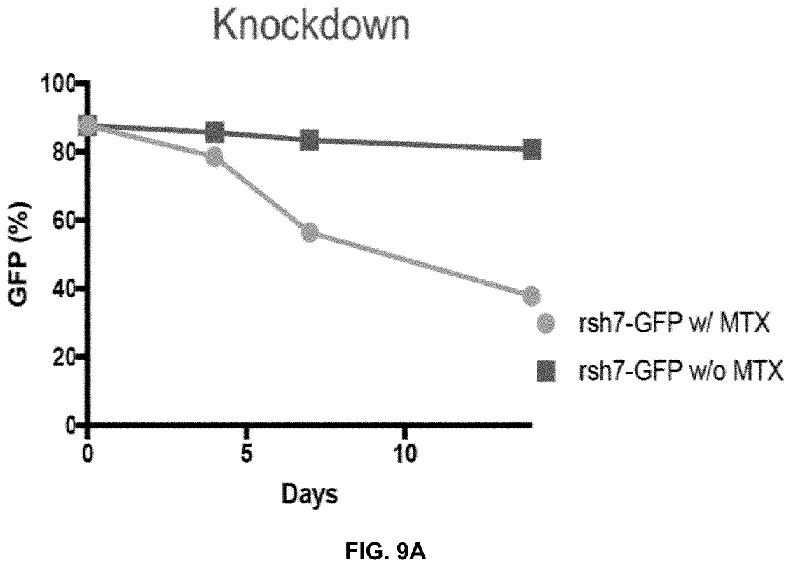

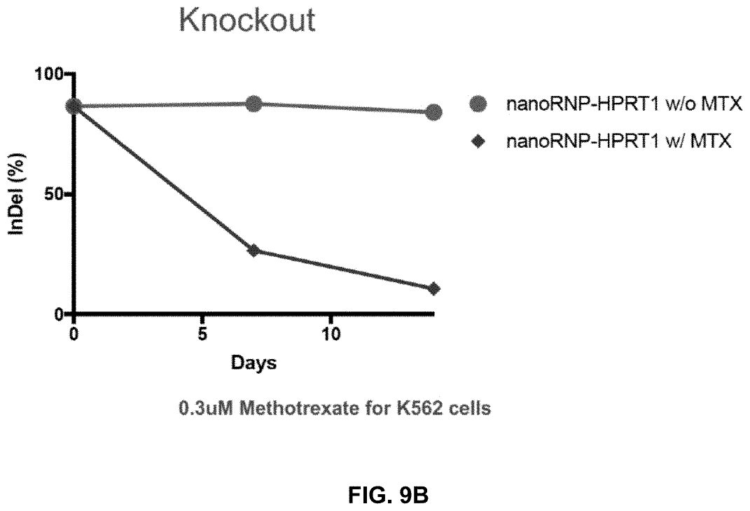

Transduced or transfected K562 cells (such as those from Example 5) were cultured with or without MTX from day 0 to day 14. The medium was refreshed every 3 to 4 days. GFP was analyzed on flow machine and InDel % was analyzed by T7E1 assay. FIG. 9A showed that the GFP-population of transduced K562 cells decreased under the treatment of 0.3 uM of MTX the population of cells was steady without MTX. FIG. 9B illustrated that the transfected K562 cells were eliminated under treatment with 0.3 uM of MTX at a faster pace as compared with the HPRT-KD population.

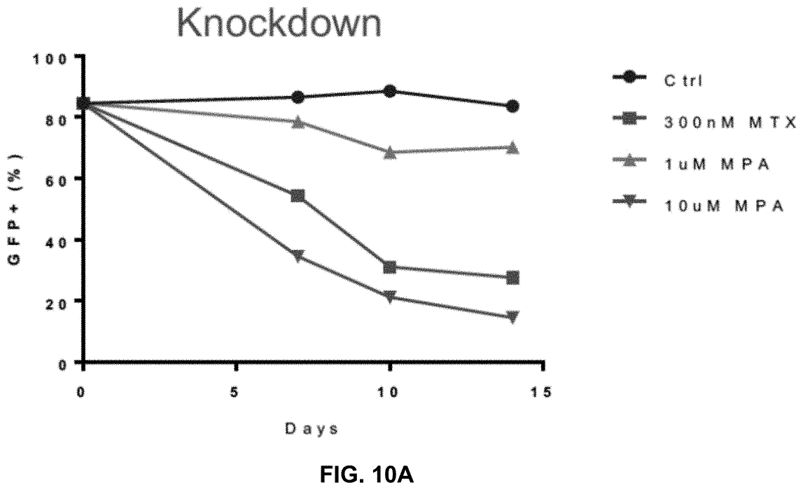

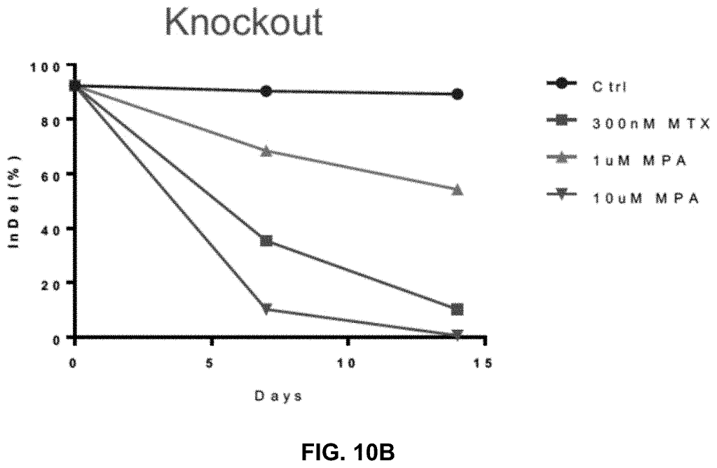

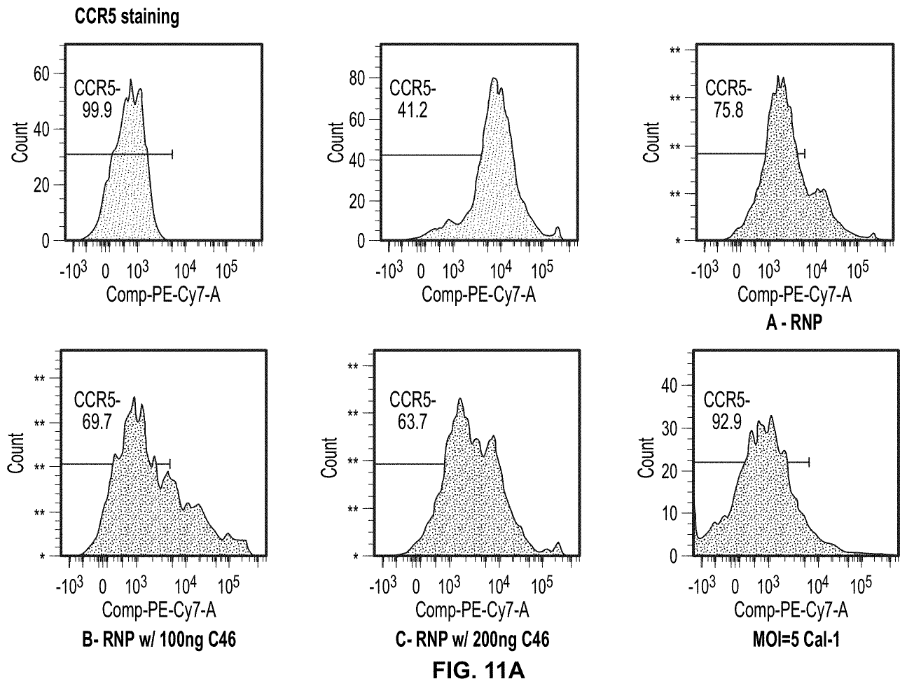

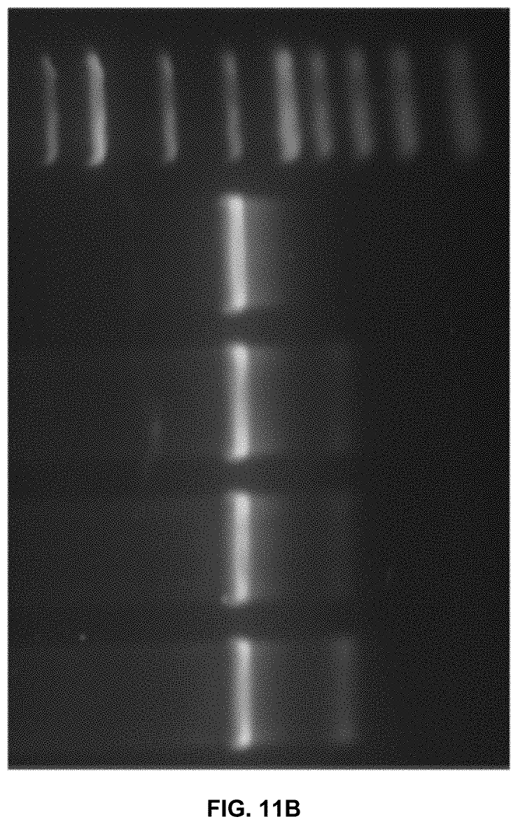

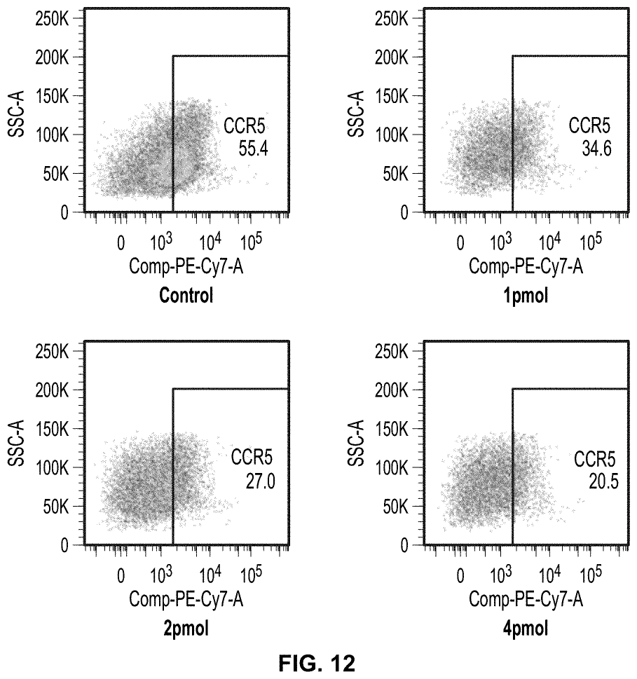

Transduced or transfected CEM cells (such as those from Example 6) were cultured with or without MTX from day 0 to day 14. The medium was refreshed every 3 to 4 days. GFP was analyzed on flow machine and InDel % was analyzed by T7E1 assay. FIG. 10A shows the GFP-population of transduced K562 decreased under the treatment of 1 uM of MPA or 0.3 uM of MTX or 10 uM of MPA while the population of cells was steady for the untreated group. FIG. 10B illustrates that the HPRT knockout population of CEM cells were eliminated at a faster pace under the treatment of 1 uM of MPA or 0.3 uM of MTX or 10 uM of MPA.

### Example 8—Preparation of ERNA and hdrDNA—Knock-Out and HDR of HBB with ERNA-Cas9-hdrDNA Nanocapsules

Method

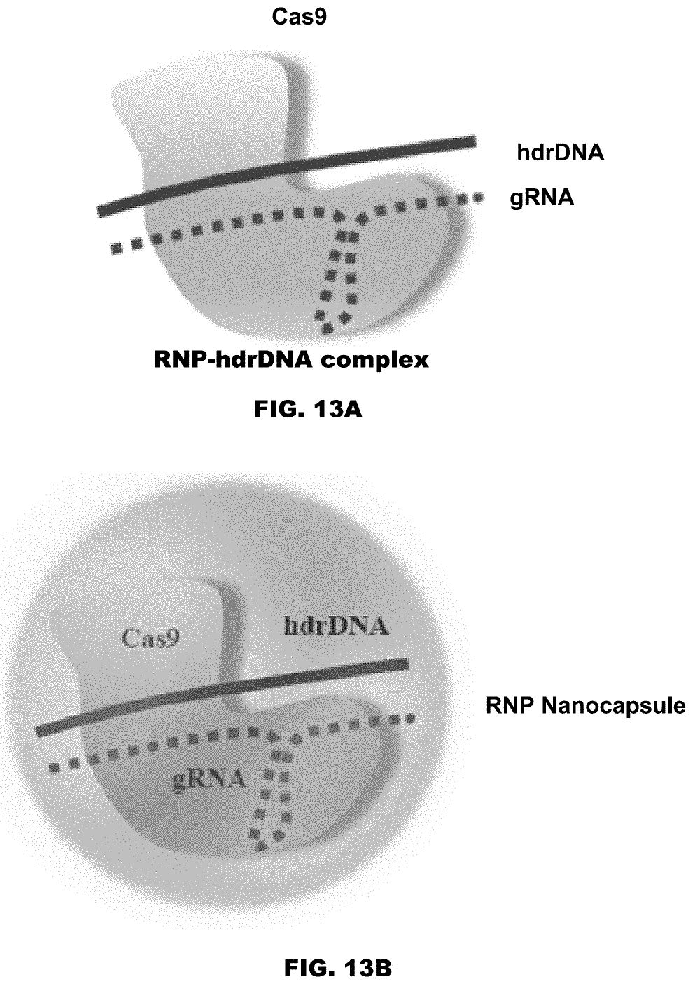

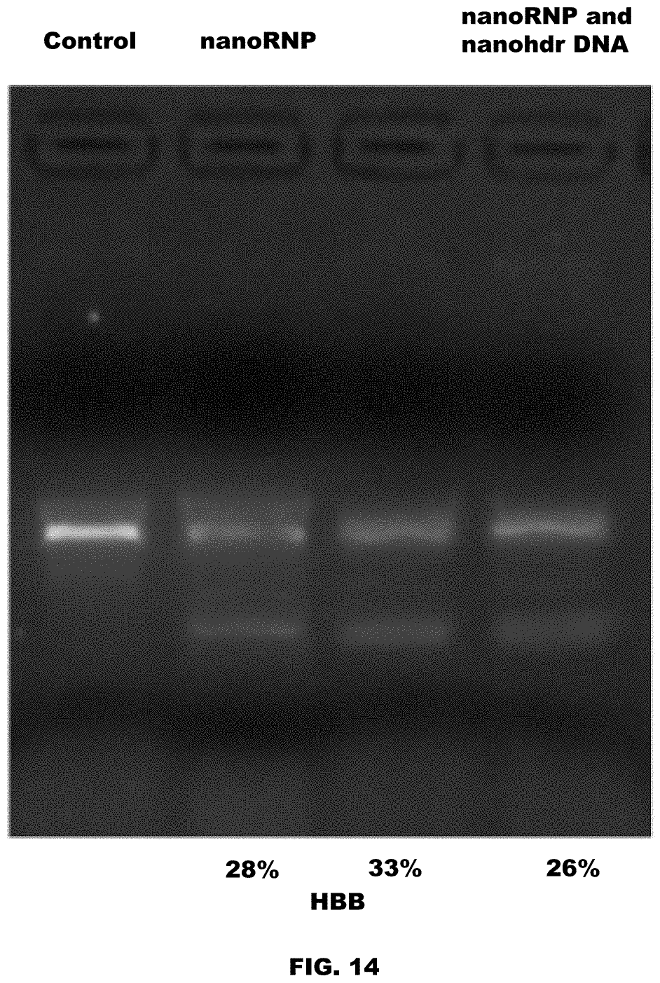

293T cells were seeded one day before transduction. 1.5 pmol of i) RNP (control), ii) RNP-hdrDNA complex, or iii) RNP and hdrDNA Nanocapsules with RNP for HBB was added into the well and incubated for 4 hours before refreshing the medium. A T7E1 assay was carried out 3 days later (see also FIGS. 13A, 13B, and 14).

The target sequence was as follows:
(SEQ ID NO: 38)
TTACTGCCCTGTGGGGCAAG.
The hdr DNA (HBB-SCD) sequence was:
(SEQ ID NO: 56)
CACTAGCAACCTCAAACAGACACCATGGTGCATCTGACTCCTGTGGAGAA
GTCTGCCGTTACTGCCCTGTGGGGCAAGGTGAACGTGGATGAAGTTGGTG.

nanon(RNP-hdrDNA) complex—gRNA Sequences are provided in the Table which follows:

| SEQ ID | |
|---|---|
| NO: | Sequence |
| 39 | GACTCCTGAGGAGAAGTCTGCCGTTACTGCCCTGTGGGG |
| | CAAGGTGAACGTG |
| 40 | GACTCCTGAGGAGAAGTCTGCCGTTATGCCCTTGGGGCA |
| | AGGTGAACGTG |
| 41 | GACTCCTGAGGAGAAGTCTGCCGTTAGCCCTGTGGGGCA |
| | AGGTGAACGTG |
| 42 | GACTCCTGAGGAGAAGTCTGCCGTTACCCTGTGGGGCAA |
| | GGTGAACGTG |
| 43 | GACTCCTGAGGAGAAGTCTGCCGTACCTGTGGGGCAAGG |
| | TGAACGTG |
| 44 | GACTCCTGAGGAGAAGTCTGCCGTT GGGGCAAGGTGAA |
| | CGTG |
| 45 | GACTCCTGTGGAGAAGTCTGCCGTTACTGCCCTGTGGGG |
| | CAAGGTGAACGTG |
| 46 | GACTCCTGTGGAGAAGTCTGCCGTTACTGCCCTGTGGGG |
| | CAAGGTGAACGTG |
| 47 | GACTCCTGTGGAGAAGTCTGCCGTTACTGCCCTGTGGGG |
| | CAAGGTGAACGTG |

INDEL %=8/23 (35% vs 33% from T7E1)

HDR %=3/8 (38%)

nanoRNP and nanohdrDNA—gRNA Sequences are provided in the Table which follows:

| SEQ ID | |
|---|---|
| NO: | Sequence |
| 48 | GACTCCTGAGGAGAAGTCTGCCGTTACTGCCCTGTGGGG |
| | CAAGGTGAACGTG |
| 49 | GACTCCTGAGGAGAAGTCTGCCGTTACCCCTGTGGGGCA |
| | AGGTGAACGTG |
| 50 | GACTCCTGAGGAGAAGTCTGCCGTTGCCCTGTGGGGCAA |
| | GGTGAACGTG |
| 51 | GACTCCTGAGGAGAAGTCTGCCGTGCCCTGTGGGGCAAG |
| | GTGAACGTG |
| 52 | GACTCCTGAGGAGAAGTCTGCCGTGCCCTGTGGGGCAAG |
| | GTGAACGTG |
| 53 | GACTCCTGAGGAGAAGTCTGCCGCCCTGTGGGGCAAGGT |
| | GAACGTG |
| 54 | GACTCCTGTGGAGAAGTCTGCCGTTACTGCCCTGTGGGG |
| | CAAGGTGAACGTG |

INDEL %=6/21 (29% vs 26% from T7E1)

HDR %=1/6 (17%)

### Example 9—Transduction/Transfection

Unless otherwise noted, transduction/transfection were conducted by standard methods. For example, refer to Yan, M. et al. (2015); PloS One.; 10(6):e0127986; Yan, M. et al. (2012). Journal of the American Chemical Society, 134, pp. 13542-13545; Zhang, J. et al. (2011). Biomacromolecules, 12 (4), pp. 1006-1014.

### Example 10—Knock-Out of CCR5 and Knock-in of C46 in MOCHA with 3-in-1 CRISPR Nanomachinery

Knock-out of CCR5 and knock-in of C46 in MOCHA (i.e. Transferrin-conjugated Cas9-CCR5-gRNA-EF1a-C46 (3-in-1) nanocapsules.

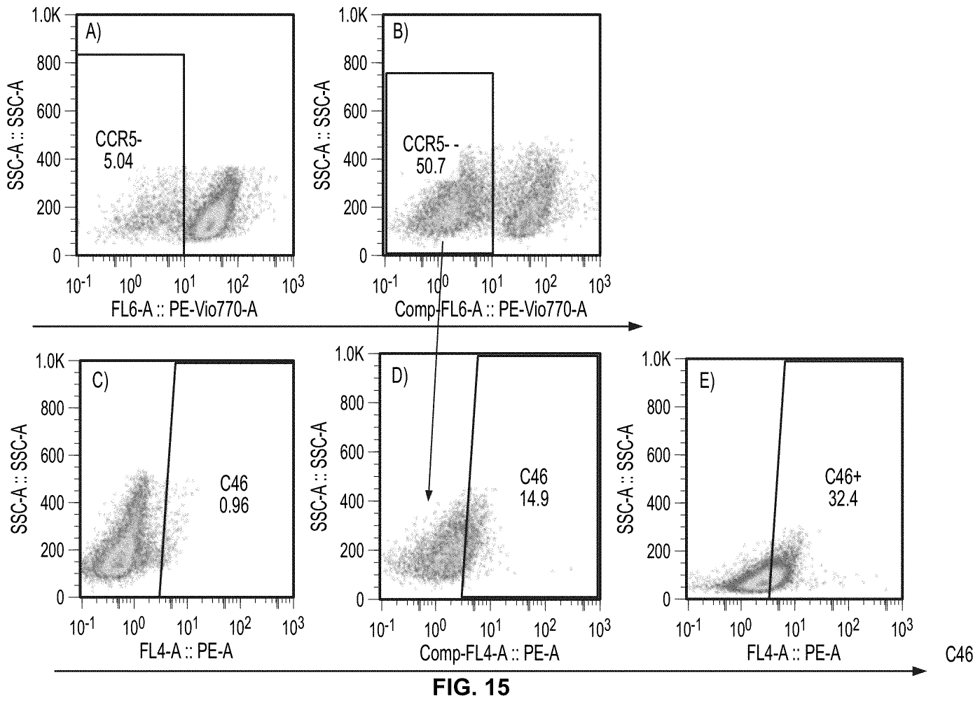

Nanocapsules were formulated to contain CRISPR/Cas9/C46 HDR template CRISPR machinery complex. Nanocapsules were also conjugated with transferrin to facilitate delivery to MOCHA cell line. 500 ng of Cas9-CCR5-gRNA-EF1a-C46 nanocapsules were added and flow analysis was carried out 3 days later. FIG. 15 panels A and B show CCR5 staining result from untreated Mocha (˜95% of CCR5+) cells and treated MOCHA. FIG. 15 panels C, D, and E show C46 staining results of untreated Mocha, CCR5− subpopulation of treated Mocha and C46-knock-in AW072 cell line.

Method:

Cas9 protein stock (67 uM), CCR5-targeting gRNA stock (100 uM) and C46-expressing DNA cassette double-stranded DNA HDR template (100 uM) were added to 50 mM pH=6.4 50 mM HEPES buffer and 150 uM NaCl to a final concentration of 1 uM. The 3-in-1 CRISPR machinery complex formed from the Cas9/gRNA/HDR template was negatively charged. Different polymer nanocapsules were formulated, each different polymer nanocapsule incorporating a gRNA having SEQ ID NO: 55.

Then, a chemical cocktail of (i) N-(3-((4-((3-aminopropyl)amino)butyl)amino)propyl)acrylamide, (ii) 2-(dimethylamino)ethyl acrylate, (iii) 1,3-glycerol dimethacrylate, and (iv) acrylamide was dissolved in deoxygenated Rnase-free water and is added to the Cas9-gRNA complex tube. The ratio of the various monomers was 750:500:1000:4000 (monomers (i):(ii):(iii):(iv)). Radical polymerization was carried out by adding 0.02 mg of ammonium persulfate dissolved in 2 μL of deoxygenated and deionized water and 0.4 μL of N,N,N′,N′-tetramethylethylenediamine. The reaction was allowed to proceed at 4° C. for 180 min in a nitrogen atmosphere. After completion of polymerization, dialysis or ultrafiltration was used to remove rid excess monomers.

Then, the nanocapsules were purified and conjugated with targeting moieties and PEG. Specifically, an about 30 to about 50 fold excess of formed nanocapsule to dibenzocyclooctyne-S—S—N-hydroxysuccinimidyl ester was used to modify the surface of nanocapsules to reach a final conjugation of between about 3 to about 10 esters per nanocapsule. Then, an about 5 to about 10 fold excess of azido functionalized PEGs having a molecular weight of about 2000 daltons, and an about 5 to about 10 fold excess of azido functionalized transferrin or abCD3 or abCD34 was used to introduce targeting moieties and stabilization moieties to the surface of the formed polymer nanocapsules. Next, the conjugated nanocapsules were separated from unreacted targeting molecules and PEG and the final product enriched using size-exclusion column chromatography. The generated polymer nanocapsule conjugates had a neutral charge and had a size ranging from between about 50 nm to about 100 nm.

### Example 11—6-TG Selection with 3-in-1 CRISPR Machinery

Assay protocols and results for CCR5 staining with 6-TG selection in sh734-KI mPB CD34+ cells (i.e. Mobilized CD34+ cells cultured with TPO/SCF/FLT3/IL3 and Knock-out of CCR5 and knock-in of sh734 in CD34+ cells)

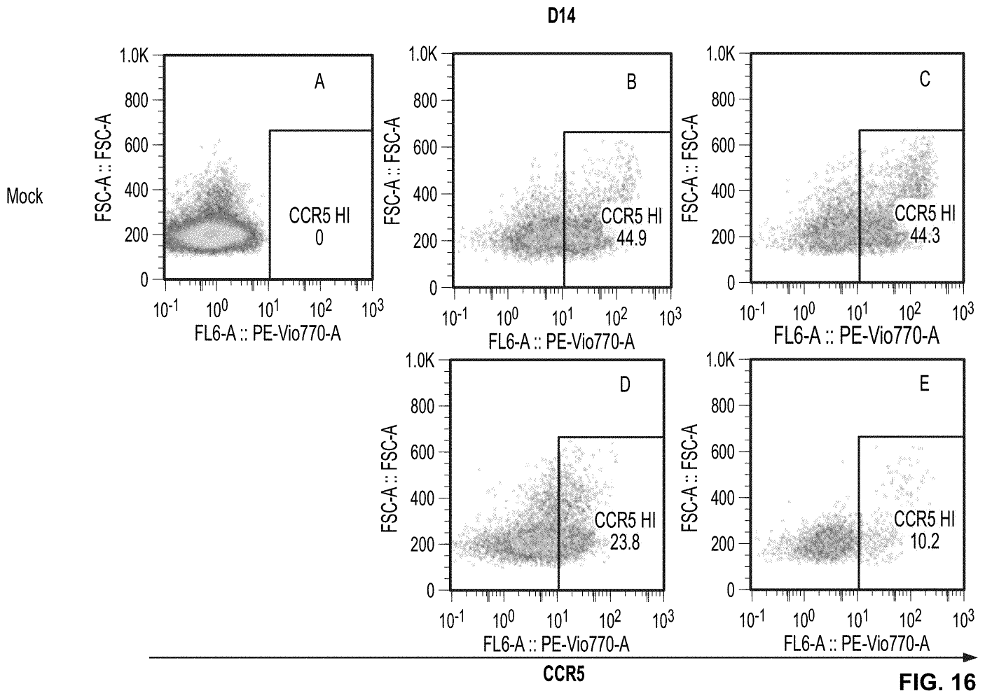

Nanocapsules were formulated to contain CRISPR/Cas9/gRNAR targeting CCR5/mi-sh734 expression cassette HDR template CRISPR machinery complex (see methods). Cd34+ cells were thawed and pre-stimulated in x-vivo 10 with 100 ng/mL of TPO/SCF/FLT4/IL3 for overnight. Then 500 ng of Cas9-CCR5-gRNA-3G-mi-sh734 nanocapsules were added into 5×10\hat{ }4 cells per well and 100 nM of 6TG was added into culture media from day 4 to 14. The flow analysis was carried out at day 14. FIG. 16 panels A, B, and C showed unstained control, CCR5 staining result from Cd34+ cells without 6TG treatment (44.9% of CCR5+) (FIG. 16, panel B) and with 6TG treatment (43.4% of CCR5+) (FIG. 16, panel C). FIG. 16 panel D showed CCR5+ stained results from nanocapsule-treated CD34+ cells without 6TG in culture media (23.8%) and with 6TG in culture media (10.2%).

CCR5 staining data suggest 6-TG selection happens for sh734-KI mPB CD34+ cells on bulk culture.

Method

Cas9 protein stock (67 uM), CCR5-targeting gRNA stock (100 uM) and 7SK-sh734-expressing DNA cassette double-stranded DNA HDR template (100 uM) were added to 50 mM pH=6.4 50 mM HEPES buffer and 150 uM NaCl to a final concentration of 1 uM. The 3-in-1 CRISPR machinery complex formed from the Cas9/gRNA/HDR template was negatively charged.

Then, a chemical cocktail of (i) N-(3-((4-((3-aminopropyl)amino)butyl)amino)propyl)acrylamide, (ii) 2-(dimethylamino)ethyl acrylate, (iii) 1,3-glycerol dimethacrylate, and (iv) acrylamide was dissolved in deoxygenated Rnase-free water and is added to the Cas9-gRNA complex tube. The ratio of the various monomers was 850:500:1000:4000 (monomers (i):(ii):(iii):(iv)). Radical polymerization was carried out by adding 0.02 mg of ammonium persulfate dissolved in 2 μL of deoxygenated and deionized water and 0.4 μL of N,N,N′,N′-tetramethylethylenediamine. The reaction was allowed to proceed at 4° C. for 180 min in a nitrogen atmosphere. After completion of polymerization, dialysis or ultrafiltration was used to remove rid excess monomers.

Then, the nanocapsules were purified and conjugated with targeting moieties and PEG. Specifically, an about 30 to about 50 fold excess of formed nanocapsule to dibenzocyclooctyne-S—S—N-hydroxysuccinimidyl ester was used to modify the surface of nanocapsules to reach a final conjugation of between about 3 to about 10 esters per nanocapsule. Then, an about 5 to about 10 fold excess of azido functionalized PEGs having a molecular weight of about 2000 daltons, and an about 5 to about 10 fold excess of azido functionalized transferrin or abCD3 or abCD34 was used to introduce targeting moieties and stabilization moieties to the surface of the formed polymer nanocapsules. Next, the conjugated nanocapsules were separated from unreacted targeting molecules and PEG and the final product enriched using size-exclusion column chromatography. The generated polymer nanocapsule conjugates had a neutral charge and had a size ranging from between about 50 nm to about 100 nm (see FIG. 16).

### Example 12-6 TG Selection with CRISPR Nanocapsules

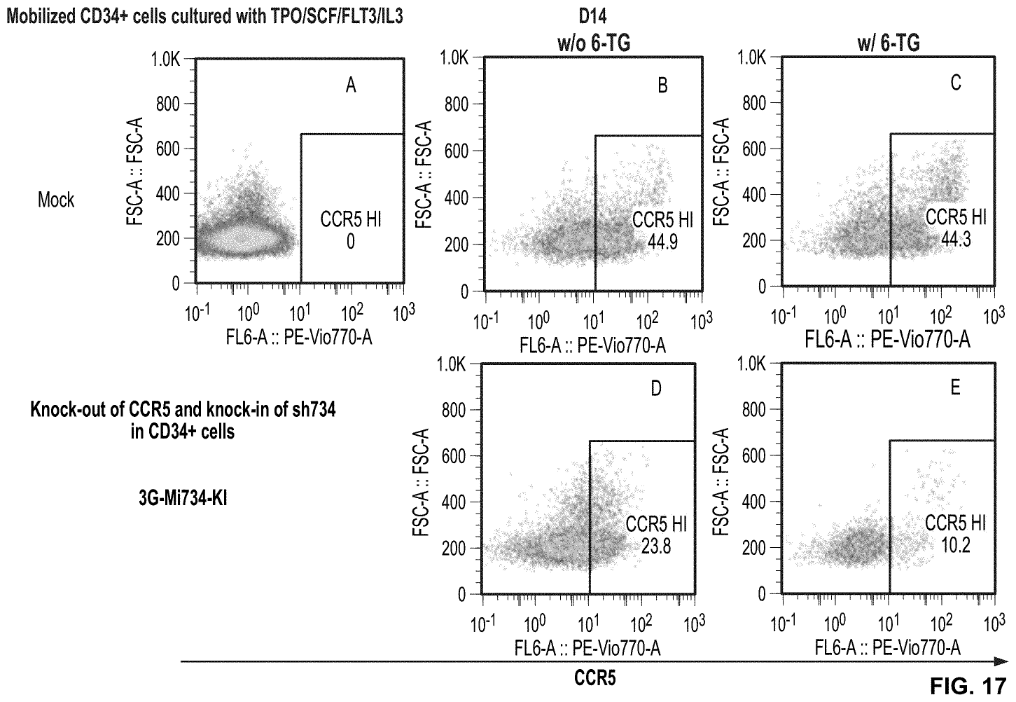

6-TG selection was examined using VCN/InDel data. mPB CD34+ were transfected/transduced as indicated in FIG. 8A Nanocapsules were formulated to contain CRISPR/Cas9/gRNAR targeting CCR5/mi-sh734 expression cassette HDR template CRISPR machinery complex (see methods). Cd34+ cells were thawed and prestimulated in X-vivo 10 with 100 ng/mL of TPO/SCF/FLT4/IL3 for overnight. Then 500 ng of Cas9-CCR5-gRNA-3G-mi-sh734 nanocapsules were added into 5×10⁴ cells per well and 100 nM of 6TG was added into culture media from day 4 to 14. The flow analysis was carried out at day 14. FIG. 17 showed unstained control, CCR5 staining result from Cd34+ cells without 6TG treatment (44.9% of CCR5+) with 6TG treatment (43.4% of CCR5+). FIG. 17 further showed CCR5+ stained results from nanocapsule-treated CD34+ cells without 6TG in culture media (23.8%) and with 6TG in culture media (10.2%). CCR5 staining data suggest 6-TG selection happens for sh734-KI mPB CD34+ cells on bulk culture.

6-TG selection for mPB CD34+ cells transduced with sh734-rGbG/rGbG-sh734/sh734-GFP vectors or HPRT-KO CD34+ cells on 6-TG-treated methylcellulose plate were examined. VCN/InDel data suggest 6-TG selection occurs for mPB CD34+ cells transduced with sh734-rGbG/rGbG-sh734/sh734-GFP vectors or HPRT-KO CD34+ cells on 6-TG-treated methylcellulose plate (see FIGS. 8D and 8E).

### Additional Embodiments

In a first additional embodiment is a polymer nanocapsule comprising a polymer shell and a payload, wherein the polymer shell comprises at least two different positively charged monomers, at least one neutral monomer, and a crosslinker; wherein the payload is selected from the group consisting of ribonucleoprotein complexes, siRNA molecules, shRNA molecules, expression vectors, polynucleotides, such as polynucleotides having between 50 and 500 base pairs, peptides, enzymes, antibodies, and antibody fragments. In some embodiments, ribonucleoprotein complex comprises an endonuclease and a guide RNA. In some embodiments, the endonuclease is a Cas protein. In some embodiments, the Cas protein is selected from the group consisting of Cas9, Cas12a, and Cas12b. In some embodiments, the guide RNA targets the HPRT locus. In some embodiments, the guide RNA that targets HPRT comprises a nucleic acid sequence having at least 90% identity to that of SEQ ID NO: 2. In some embodiments, the guide RNA that targets HPRT comprises a nucleic acid sequence having at least 95% identity to that of SEQ ID NO: 2. In some embodiments, the guide RNA targets the beta-globin locus. In some embodiments, the guide RNA that targets beta-globin comprises a nucleic acid sequence having at least 90% identity to that of SEQ ID NO: 3. In some embodiments, the guide RNA that targets beta-globin comprises a nucleic acid sequence having at least 95% identity to that of SEQ ID NO: 3.

In some embodiments, the positively charged monomers are selected from the group consisting of:
N-(3-((4-((3-aminopropyl)amino)butyl)amino) propyl)acrylamide,
N-(3-((4-((3-aminopropyl)amino)butyl)amino)propyl)methacrylamide,
N-(3-((4-aminobutyl)amino)propyl)acrylamide,
N-(3-((4-aminobutyl)amino)propyl)methacrylamide,
N-(2-((2-aminoethyl)(methyl)amino)ethyl)acrylamide,
N-(2-((2-aminoethyl)(methyl)amino)ethyl)methacrylamide,
N-(piperazin-1-ylmethyl)acrylamide,
N-(piperazin-1-ylmethyl)methacrylamide,
N-(2-(bis(2-aminoethyl)amino)ethyl)acrylamide,
N-(2-(bis(2-aminoethyl)amino)ethyl)methacrylamide,
N-(3-Aminopropyl)methacrylamidehydrochloride,
2-(dimethylamino)ethyl acrylate,
Dimethylamino ethyl methacrylate,
(3-Acrylamidopropyl) trimethylammonium hydrochloride,
2-aminoethyl methacrylate,
3-(Dimethylamino)propyl acrylate,
N-[3-(Dimethylamino)propyl]methacrylamide, and any combination thereof.

In some embodiments, the neutral monomer is selected from N-(1,3-dihydroxy-2-(hydroxymethyl)propan-2-yl)acrylamide, acrylamide, N-(hydroxymethyl)acrylamide, 2-hydroxyethyl acrylate, and 2-hydroxyethyl methacrylate. In some embodiments, the crosslinkers are selected from the group consisting of 1,3-glycerol dimethacrylate, N,N′-Methylenebisacrylamide, and glycerol 1,3-diglycerolate diacrylate.

In some embodiments, the polymer nanocapsule further comprises at least one targeting moiety. In some embodiments, the polymer nanocapsule comprises between 2 and between 6 targeting moieties. In some embodiments, the at least one targeting moiety is an antibody. In some embodiments, the at least one targeting moiety is an antibody and wherein the polymer nanocapsule comprises between 1 and 3 antibodies. In some embodiments, the polymer nanocapsule comprises at least one stabilizing moiety. In some embodiments, the at least one stabilizing moiety comprises at least one polyethylene glycol group. In some embodiments the polymer nanocapsule comprises at least one targeting moiety and at least one stabilizing moiety. In some embodiments, the polymer nanocapsule comprises between 2 and 6 targeting moieties and between at least 2 and 6 stabilizing moieties. In some embodiments, the at least one targeting moiety is an antibody, and the stabilizing moiety comprises at least one polyethylene glycol group.

In some embodiments, the polymer shell is free from monomers or crosslinkers including a heterocyclic group. In some embodiments, the polymer shell is free from monomers or crosslinkers including an imidazole group. In some embodiments, the polymer shell is free of imidazolyl acryloyl monomers.

In a second additional embodiment is a method of modifying a target nucleic acid sequence within a host cell comprising: contacting the host cell with one or more polymer nanocapsules, wherein the one or more polymer nanocapsules comprise a polymer shell and a payload and wherein the polymer shell comprises at least two different positively charged monomers, at least one neutral monomer, and a crosslinker; wherein the payload is selected from the group consisting of ribonucleoprotein complexes, siRNA molecules, shRNA molecules, expression vectors, polynucleotides, such as polynucleotides having between 50 and 500 base pairs, peptides, enzymes, antibodies, and antibody fragments. In some embodiments, the host cells are hematopoietic stem cells. In some embodiments, the hematopoietic stem cells are allogenic hematopoietic stem cells. In some embodiments, the hematopoietic stem cells are autologous hematopoietic stem cells. In some embodiments, the hematopoietic stem cells are sibling matched hematopoietic stem cells.

In some embodiments, ribonucleoprotein complex comprises an endonuclease and a guide RNA. In some embodiments, the endonuclease is a Cas protein. In some embodiments, the Cas protein is selected from the group consisting of Cas9, Cas12a, and Cas12b. In some embodiments, the guide RNA targets the HPRT locus. In some embodiments, the guide RNA that targets HPRT comprises a nucleic acid sequence having at least 90% identity to that of SEQ ID NO: 2. In some embodiments, the guide RNA that targets HPRT comprises a nucleic acid sequence having at least 95% identity to that of SEQ ID NO: 2. In some embodiments, the guide RNA targets the beta-globin locus. In some embodiments, the guide RNA that targets beta-globin comprises a nucleic acid sequence having at least 90% identity to that of SEQ ID NO: 3. In some embodiments, the guide RNA that targets beta-globin comprises a nucleic acid sequence having at least 95% identity to that of SEQ ID NO: 3. In some embodiments, the polymer nanocapsule is free from monomers or crosslinkers including a heterocyclic group. In some embodiments, the polymer nanocapsule is free from monomers or crosslinkers including an imidazole group.

In a third additional embodiment is a method of providing benefits of a lymphocyte infusion to a patient in need of treatment thereof while mitigating side effects comprising: generating HPRT deficient lymphocytes from a donor sample, wherein the HPRT deficient lymphocytes are generating by transfecting lymphocytes within the donor sample with a nanocapsule including components adapted to knockout HPRT (e.g. a Cas9 or Cas12a protein and a gRNA targeting a portion of the HPRT gene); positively selecting for the HPRT deficient lymphocytes ex vivo to provide a population of modified lymphocytes; administering an HSC graft to the patient; administering the population of modified lymphocytes to the patient following the administration of the HSC graft; and optionally administering a dihydrofolate reductase inhibitor if the side effects arise. In some embodiments, the dihydrofolate reductase inhibitor is selected from the group consisting of methotrexate (MTX) or mycophenolic acid (MPA). In some embodiments, the positive selection comprises contacting the generated HPRT deficient lymphocytes with a purine analog. In some embodiments, the purine analog is 6TG. In some embodiments, an amount of 6TG ranges from between about 1 to about 15 μg/mL. In some embodiments, the positive selection comprises contacting the generated HPRT deficient lymphocytes with both a purine analog and allopurinol. In some embodiments, the modified lymphocytes are administered as a single bolus. In some embodiments, multiple doses of the modified lymphocytes are administered to the patient. In some embodiments, each dose of the modified lymphocytes comprises between about 0.1×10⁶ cells/kg to about 240×10⁶ cells/kg. In some embodiments, a total dosage of modified lymphocytes comprises between about 0.1×10⁶ cells/kg to about 730×10⁶ cells/kg.

In a fourth additional embodiment is a polymer nanocapsule comprising a polymer shell and a ribonucleoprotein complex. In some embodiments, the ribonucleoprotein comprises an endonuclease and a guide RNA. In some embodiments, the endonuclease is a Cas protein. In some embodiments, the Cas protein is Cas9. In some embodiments, the Cas protein is Cas12. In some embodiments, the guide RNA targets the HPRT locus. In some embodiments, the guide RNA a targets a nucleic acid sequence within a beta-globin gene. In some embodiments, the guide RNA a targets a BCL11A binding site in a regulatory region of a gamma-globin promoter. In some embodiments, the polymer nanocapsule further comprises at least one targeting moiety to facilitate delivery of the ribonucleoprotein complex to a particular type of cell (e.g. a targeting moiety conjugated to the surface of the polymer nanocapsule). In some embodiments, the polymer nanocapsule is erodible or biodegradable. In some embodiments, the polymer nanocapsule includes a pH sensitive crosslinker. In some embodiments, the polymer nanocapsule has a size ranging from between about 50 nm to about 250 nm. In some embodiments, the polymer shell comprises one positively charged monomer. In some embodiments, the polymer shell comprises two positively charged monomers.

In some embodiments is a polymer nanocapsule comprising a polymer shell and a ribonucleoprotein complex, wherein the polymer shell comprises (i) at least one of N-(3-((4-((3-aminopropyl)amino)butyl)amino)propyl)acrylamide or 2-(dimethylamino)ethyl acrylate; (ii) acrylamide or a derivative thereof; and (iii) a crosslinker (e.g. a pH degradable crosslinker). In some embodiments, the polymer shell comprises both N-(3-((4-((3-aminopropyl)amino)butyl)amino)propyl)acrylamide and 2-(dimethylamino)ethyl acrylate. In some embodiments, the crosslinker is an acrylate. In some embodiments, the acrylate is 2-(dimethylamino)ethyl acrylate.

In some embodiments, the polymer nanocapsule further comprises at least one targeting moiety. In some embodiments, the targeting moiety is coupled to the polymer nanocapsule through a spacer including a cleavable group, e.g. a disulfide bond. In some embodiments, the targeting moiety is an antibody. In some embodiments, the targeting moiety facilitates the delivery of the polymer nanocapsule to a specific cell type, wherein the cell type is selected from the group comprising immune cells, blood cells, cardiac cells, lung cells, optic cells, liver cells, kidney cells, brain cells, cells of the central nervous system, cells of the peripheral nervous system, cancer cells, cells infected with viruses, stem cells, skin cells, intestinal cells, and/or auditory cells. In some embodiments, the cancer cells are cells selected from the group comprising lymphoma cells, solid tumor cells, leukemia cells, bladder cancer cells, breast cancer cells, colon cancer cells, rectal cancer cells, endometrial cancer cells, kidney cancer cells, lung cancer cells, melanoma cells, pancreatic cancer cells, prostate cancer cells, and thyroid cancer cells.

In some embodiments, the polymer nanocapsule further comprises at least one stabilizing moiety. In some embodiments, the at least one stabilizing moiety includes at least one alkylene oxide group, e.g. a polyethylene glycol repeat group, and/or a polypropylene glycol repeat group. In some embodiments, at least four polyethylene glycol repeat groups and/or polypropylene glycol repeat groups are included within the at least one stabilizing moiety.

In some embodiments, the ribonucleoprotein complex includes a guide RNA that targets the HPRT gene. In some embodiments, the ribonucleoprotein complex includes a guide RNA that targets a beta-globin gene. In some embodiments, the ribonucleoprotein complex includes a guide RNA that targets a BCL11A binding site in a regulatory region of a gamma-globin promoter. In some embodiments, the polymer nanocapsule has a diameter ranging from between about 50 nm to about 250 nm.

In a fifth additional embodiment is a polymer nanocapsule comprising a polymer shell and a ribonucleoprotein complex, wherein the polymer shell comprises at least two different positively charged monomers, at least one neutral monomer, and a crosslinker. In some embodiments, ribonucleoprotein complex comprises an endonuclease and a guide RNA. In some embodiments, the endonuclease is a Cas protein. In some embodiments, the Cas protein is selected from the group consisting of Cas9, Cas12a, and Cas12b. In some embodiments, the guide RNA targets the HPRT locus. In some embodiments, the guide RNA that targets HPRT comprises a nucleic acid sequence having at least 90% identity to that of SEQ ID NO: 2. In some embodiments, the guide RNA that targets HPRT comprises a nucleic acid sequence having at least 95% identity to that of SEQ ID NO: 2. In some embodiments, the guide RNA targets the beta-globin locus. In some embodiments, the guide RNA that targets beta-globin comprises a nucleic acid sequence having at least 90% identity to that of SEQ ID NO: 3. In some embodiments, the guide RNA that targets beta-globin comprises a nucleic acid sequence having at least 95% identity to that of SEQ ID NO: 3.

In some embodiments, the positively charged monomers are selected from the group consisting of:
N-(3-((4-((3-aminopropyl)amino)butyl)amino) propyl)acrylamide,
N-(3-((4-((3-aminopropyl)amino)butyl)amino)propyl)methacrylamide,
N-(3-((4-aminobutyl)amino)propyl)acrylamide,
N-(3-((4-aminobutyl)amino)propyl)methacrylamide,
N-(2-((2-aminoethyl)(methyl)amino)ethyl)acrylamide,
N-(2-((2-aminoethyl)(methyl)amino)ethyl)methacrylamide,
N-(piperazin-1-ylmethyl)acrylamide,
N-(piperazin-1-ylmethyl)methacrylamide,
N-(2-(bis(2-aminoethyl)amino)ethyl)acrylamide,
N-(2-(bis(2-aminoethyl)amino)ethyl)methacrylamide,
N-(3-Aminopropyl)methacrylamidehydrochloride,
2-(dimethylamino)ethyl acrylate,
Dimethylamino ethyl methacrylate,
(3-Acrylamidopropyl) trimethylammonium hydrochloride,
2-aminoethyl methacrylate,
3-(Dimethylamino)propyl acrylate,
N-[3-(Dimethylamino)propyl]methacrylamide, and any combination thereof.

In some embodiments, the neutral monomer is selected from N-(1,3-dihydroxy-2-(hydroxymethyl)propan-2-yl)acrylamide, acrylamide, N-(hydroxymethyl)acrylamide, 2-hydroxyethyl acrylate, and 2-hydroxyethyl methacrylate. In some embodiments, the crosslinkers are selected from the group consisting of 1,3-glycerol dimethacrylate, N,N′-Methylenebisacrylamide, and glycerol 1,3-diglycerolate diacrylate.

In some embodiments, the polymer nanocapsule further comprises at least one targeting moiety. In some embodiments, the polymer nanocapsule comprises between 2 and between 6 targeting moieties. In some embodiments, the at least one targeting moiety is an antibody. In some embodiments, the at least one targeting moiety is an antibody and wherein the polymer nanocapsule comprises between 1 and 3 antibodies. In some embodiments, the polymer nanocapsule comprises at least one stabilizing moiety. In some embodiments, the at least one stabilizing moiety comprises at least one polyethylene glycol group. In some embodiments the polymer nanocapsule comprises at least one targeting moiety and at least one stabilizing moiety. In some embodiments, the polymer nanocapsule comprises between 2 and 6 targeting moieties and between at least 2 and 6 stabilizing moieties. In some embodiments, the at least one targeting moiety is an antibody, and the stabilizing moiety comprises at least one polyethylene glycol group.

In some embodiments, the polymer shell is free from monomers or crosslinkers including a heterocyclic group. In some embodiments, the polymer shell is free from monomers or crosslinkers including an imidazole group. In some embodiments, the polymer shell is free of imidazolyl acryloyl monomers.

In a sixth additional embodiment is a conjugate comprising: (i) a polymer nanocapsule, wherein the polymer nanocapsule comprises a polymer shell and a ribonucleoprotein complex, wherein the polymer shell comprises at least two positively charged monomers, at least one neutral monomer, and a crosslinker; and (ii) at least one of (a) a targeting moiety, or (b) a stabilizing moiety, coupled to the polymer nanocapsule.

In a seventh additional embodiment is a method of treating a genetic condition within a human patient comprising: generating a population of modified human hematopoietic stem cells by contacting an unmodified population of human hematopoietic stem cells with a polymer nanocapsule, the polymer nanocapsule including a polymer shell and a ribonucleoprotein complex, wherein the polymer shell comprises at least two positively charged monomers, at least one neutral monomer, and a crosslinker; and administering a therapeutically effective amount of the population of modified human hematopoietic stem cells to the human patient.

In an eight additional embodiment is a conjugate prepared according to a process comprising: derivatizing a polymer nanocapsule with a first reactive functional group capable of participating in a click chemistry reaction, the polymer nanocapsule having a polymer shell and a ribonucleoprotein complex, wherein the polymer shell comprises at least two positively charged monomers, at least one neutral monomer, and a crosslinker; derivatizing a targeting moiety with a second reactive functional group capable of participating in a click chemistry reaction with the first reactive functional group; and reacting the derivatized polymer nanocapsule with the derivatized targeting moiety.

In a ninth additional embodiment is a conjugate prepared according to a process comprising reacting a derivatized polymer nanocapsule with at least one derivatized targeting moiety, the derivatized polymer nanocapsule including at least one first reactive functional group capable of participating in a click chemistry reaction, the polymer nanocapsule having a polymer shell having at least two positively charged monomers, at least one neutral monomer, and a crosslinker; and wherein the at least one derivatized targeting moiety includes a second reactive functional group capable of participating in a click chemistry reaction with the first reactive functional group.
Further Embodiment 1. A polymer nanocapsule comprising a polymer shell and a ribonucleoprotein complex, wherein the polymer shell comprises at least one positively charged monomer, at least one neutral monomer, and a crosslinker.
Further Embodiment 2. The polymer nanocapsule of further embodiment 1, wherein the ribonucleoprotein complex comprises an endonuclease and a guide RNA.
Further Embodiment 3. The polymer of further embodiment 2, wherein the endonuclease is a Cas protein.
Further Embodiment 4. The polymer of further embodiment 3, wherein the Cas protein is a Class 2 Cas protein.
Further Embodiment 5. The polymer of further embodiment 4, wherein the Class 2 Cas protein is a Type II or a Type V Cas protein.
Further Embodiment 6. The polymer nanocapsule of any one of further embodiments 3 to 5, wherein the Cas protein is selected from the group consisting of Cas9 and Cas12a.
Further Embodiment 7. The polymer nanocapsule of any one of further embodiments 2-6, wherein the guide RNA targets the HPRT locus.
Further Embodiment 8. The polymer nanocapsule of further embodiment 7, wherein the guide RNA that targets the HPRT locus comprises a nucleic acid sequence having at least 90% identity to that of SEQ ID NO: 2.
Further Embodiment 9. The polymer nanocapsule of any one of further embodiments 2-6, wherein the guide RNA targets the beta-globin locus.
Further Embodiment 10. The polymer nanocapsule of further embodiment 9, wherein the guide RNA that targets the beta-globin locus comprises a nucleic acid sequence having at least 90% identity to that of SEQ ID NO: 3.
Further Embodiment 11. The polymer nanocapsule of any one of further embodiments 2-6, wherein the guide RNA targets the CCR5 locus.
Further Embodiment 12. The polymer nanocapsule of further embodiment 11, wherein the guide RNA that targets the CCR5 locus comprises a nucleic acid sequence having at least 90% identity to that of SEQ ID: 1.
Further Embodiment 13. The polymer nanocapsule of any one of the preceding further embodiments, further comprising at least one targeting moiety.
Further Embodiment 14. The polymer nanocapsule of further embodiment 13, wherein the polymer nanocapsule comprises between 2 and between 6 targeting moieties.
Further Embodiment 15. The polymer nanocapsule of further embodiment 14, wherein the targeting moieties comprise two more different targeting moieties.
Further Embodiment 16. The polymer nanocapsule of further embodiment 13, wherein the at least one targeting moiety is an antibody and wherein the polymer nanocapsule comprises between 1 and 3 antibodies.
Further Embodiment 17. The polymer nanocapsule of any one of the preceding further embodiments, further comprising at least one stabilizing moiety.
Further Embodiment 18. The polymer nanocapsule of further embodiment 17, wherein the at least one stabilizing moiety comprises at least one polyethylene glycol group.
Further Embodiment 19. The polymer nanocapsule of any one of the preceding further embodiments, wherein the polymer nanocapsule comprises at least one targeting moiety and at least one stabilizing moiety.
Further Embodiment 20. The polymer nanocapsule of further embodiment 19, wherein the targeting moiety is an antibody and wherein the stabilizing moiety comprises at least one polyethylene glycol group.
Further Embodiment 21. The polymer nanocapsule of any one of the preceding further embodiments, wherein the polymer shell comprises at least two positively charged monomers.
Further Embodiment 22. A composition comprising the polymer nanocapsule of any one of further embodiments 1 to 21, and a pharmaceutically acceptable carrier or excipient.
Further Embodiment 23. A modified host cell prepared by contacting an unmodified host cell with the polymer nanocapsules of any one of further embodiments 1 to 21 or the composition of further embodiment 22.
Further Embodiment 24. The modified host cell of further embodiment 23, wherein the unmodified host cell is a hematopoietic stem cell, optionally a CD34+ hematopoietic stem cell.
Further Embodiment 25. The modified host cell of further embodiment 24, wherein the hematopoietic stem cell is an allogenic hematopoietic stem cell.
Further Embodiment 26. The modified host cell of further embodiment 24, wherein the hematopoietic stem cell is an autologous hematopoietic stem cell.
Further Embodiment 27. The modified host cell of further embodiment 24, wherein the hematopoietic stem cell is a sibling matched hematopoietic stem cell.
Further Embodiment 28. A conjugate prepared according to a process comprising:
   (a) synthesizing a polymer nanocapsule having a polymer shell and a ribonucleoprotein complex, wherein the polymer shell comprises at least one positively charged monomer, at least one neutral monomer, and a crosslinker;
   (b) functionalizing the surface of the polymer nanocapsule with a targeting moiety; to provide a polymer nanocapsule conjugate.
Further Embodiment 29. A conjugate prepared according to a process comprising:
   (c) derivatizing a polymer nanocapsule with a first reactive functional group capable of participating in a click chemistry reaction, the polymer nanocapsule having a polymer shell and a ribonucleoprotein complex, wherein the polymer shell comprises at least one positively charged monomer, at least one neutral monomer, and a crosslinker;
   (d) derivatizing a targeting moiety with a second reactive functional group capable of participating in a click chemistry reaction with the first reactive functional group; and
   (e) reacting the derivatized polymer nanocapsule with the derivatized targeting moiety.
Further Embodiment 30. The conjugate of further embodiment 29, wherein the targeting moiety is derivatized by reacting the targeting moiety with a compound having Formula (IIIA):

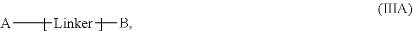

wherein
A is maleimide-C(O)—,
“Linker” is a branched or unbranched, linear or cyclic, substituted or unsubstituted, saturated or unsaturated, group having between 2 and 40 carbon atoms, and optionally having one or more heteroatoms selected from O, N, or S, and
B is the second reactive functional group and is selected from the group consisting of dibenzocyclooctyne (“DBCO”), trans-cyclooctene (“TCO”), azide, tetrazine, maleimide, thiol, 1,3-nitrone, aldehyde, ketone, hydrazine, and hydroxylamine.
Further Embodiment 31. The conjugate of further embodiment 30, wherein the “Linker” has the structure of Formula (IIIB):

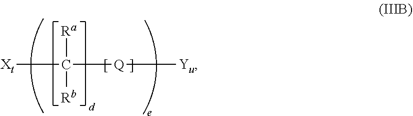

wherein d and e each independently are integers ranging from 2 to 10; t and u are independently 0 or 1; Q is a bond, O, S, or N(R^{c})(R^{d}); R^{a} and R^{b} are independently H, a C₁-C₄ alkyl group, or a halogen; R^{c} and R^{d} are independently CH₃ or H; and X and Y are independently a branched or unbranched, saturated or unsaturated group having between 1 and 4 carbon atoms and optionally having one or more O, N, or S heteroatoms.
Further Embodiment 32. The conjugate of any one of further embodiments 29-31, wherein the derivatized polymer nanocapsule is further reacted with a stabilizing moiety having Formula (V):

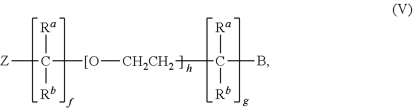

wherein B is the second reactive functional group and is selected from the group consisting of dibenzocyclooctyne (“DBCO”), trans-cyclooctene (“TCO”), azide, tetrazine, maleimide, thiol, 1,3-nitrone, aldehyde, ketone, hydrazine, and hydroxylamine.
Z is a hydroxyl group, a branched or unbranched C₁-C₄ alkyl group, —O— alkyl, —NH₂;
f and g are independently 0 or an integer ranging from 1 to 4; and
h is an integer ranging from 1 to 24.
Further Embodiment 33. The conjugate of any one of further embodiments 29-32, wherein the polymer nanocapsule is derivatized with at least three of the first reactive functional groups.
Further Embodiment 34. The conjugate of any one of further embodiments 29-33, wherein the first reactive functional group is selected from the group consisting of DBCO TCO, maleimide, an aldehyde, a ketone, an azide, a tetrazine, a thiol, a 1,3-nitrone, a hydrazine, and hydroxylamine.
Further Embodiment 35. The conjugate of any one of further embodiments 29-34, wherein the first reactive functional group comprises DBCO, and wherein the second reactive functional group comprises an azide group.
Further Embodiment 36. The conjugate of any one of further embodiments 29-35, wherein the targeting moiety comprises an antibody.
Further Embodiment 37. The conjugate of further embodiment 36, wherein the antibody is selected from the group consisting of an anti-CD4 antibody, an anti-CD8 antibody, and an anti-CD45 antibody.
Further Embodiment 38. The conjugate of any one of further embodiments 29-37, wherein the polymer nanocapsule with the first reactive functional group by reacting the polymer nanocapsule with a compound comprising a disulfide group.
Further Embodiment 39. The conjugate of any one of further embodiments 29-38, wherein the polymer nanocapsule is derivatized with the first reactive functional group by reacting the polymer nanocapsule with a compound having Formula (IVA):

A-Spacer-B (IVA),
wherein A is maleimide-C(O)—,
“Spacer” is a branched or unbranched, linear or cyclic, substituted or unsubstituted, saturated or unsaturated, group having between 2 and 20 carbon atoms, and having a disulfide bond; and
B is selected from the group consisting of dibenzocyclooctyne (“DBCO”), trans-cyclooctene (“TCO”), azide, tetrazine, maleimide, thiol, 1,3-nitrone, aldehyde, ketone, hydrazine, and hydroxylamine.
Further Embodiment 40. The conjugate of further embodiment 39, wherein the ‘Spacer’ has the structure of Formula (IVB):

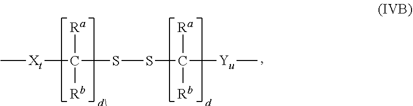

wherein
wherein d is an integer ranging from 2 to 10; t and u are independently 0 or 1; R^{a} and R^{b} are independently H, a C₁-C₄ alkyl group, or a halogen; and X and Y are independently a branched or unbranched, saturated or unsaturated group having between 1 and 8 carbon atoms and optionally having one or more O, N, or S heteroatoms.
Further Embodiment 41. The conjugate of any one of further embodiments 29-40, wherein the conjugate is targeted to a cell having a cluster of differentiation markers selected from the group consisting of CD3, CD4, CD8, and CD45.
Further Embodiment 42. The conjugate of any one of further embodiments 29-41, wherein the ribonucleoprotein complex comprises a Cas protein and a guide RNA.
Further Embodiment 43. The conjugate of further embodiment 42, wherein the guide RNA targets the HPRT locus.
Further Embodiment 44. The conjugate of further embodiment 43, wherein the guide RNA that targets the HPRT locus comprises a nucleic acid sequence having at least 90% identity to that of SEQ ID NO: 2.
Further Embodiment 45. The conjugate of further embodiment 42, wherein the guide RNA targets the beta-globin locus.
Further Embodiment 46. The conjugate of further embodiment 45, wherein the guide RNA that targets the beta-globin locus comprises a nucleic acid sequence having at least 90% identity to that of SEQ ID NO: 3.
Further Embodiment 47. The conjugate of further embodiment 42 wherein the guide RNA targets the CCR5 locus.
Further Embodiment 48. The conjugate of further embodiment 47, wherein the guide RNA that targets the CCR5 locus comprises a nucleic acid sequence having at least 90% identity to that of SEQ ID: 1.
Further Embodiment 49. A composition comprising the conjugate of any one of further embodiments 29 to 48, and a pharmaceutically acceptable carrier or excipient.
Further Embodiment 50. A modified host cell prepared by contacting a host cell with the conjugates of any one of further embodiments 29 to 48 or the composition of further embodiment 49.
Further Embodiment 51. The modified host cell of further embodiment 50, wherein the host cell is a hematopoietic stem cell.
Further Embodiment 52. The modified host cell of further embodiment 51, wherein the hematopoietic stem cell is an allogenic hematopoietic stem cell.
Further Embodiment 53. The modified host cell of further embodiment 51, wherein the hematopoietic stem cell is an autologous hematopoietic stem cell.
Further Embodiment 54. The modified host cell of further embodiment 51, wherein the hematopoietic stem cell is a sibling matched hematopoietic stem cell.
Further Embodiment 55. A method of treating a genetic condition in a human subject comprising administering to the human subject a therapeutically effective amount of the modified host cells of any one of further embodiments 50 to 54.
Further Embodiment 56. A polymer nanocapsule comprising a polymer shell and a ribonucleoprotein complex, wherein the polymer shell comprises (i) at least one of N-(3-((4-((3-aminopropyl)amino)butyl)amino)propyl)acrylamide or 2-(dimethylamino)ethyl acrylate; (ii) acrylamide or a derivative thereof; and (iii) a crosslinker.
Further Embodiment 57. The polymer nanocapsule of further embodiment 56, wherein the polymer shell comprises both N-(3-((4-((3-aminopropyl)amino)butyl)amino)propyl)acrylamide and 2-(dimethylamino)ethyl acrylate.
Further Embodiment 58. The polymer nanocapsule of any one of further embodiments 56-57, wherein the crosslinker is an acrylate.
Further Embodiment 59. The polymer nanocapsule of further embodiment 58, wherein the acrylate is 2-(dimethylamino)ethyl acrylate.
Further Embodiment 60. The polymer nanocapsule of any one of further embodiments 56-59, wherein the polymer nanocapsule further comprises at least one targeting moiety.
Further Embodiment 61. The polymer nanocapsule of further embodiment 60, wherein the at least one targeting moiety is coupled to the polymer nanocapsule through a spacer including a disulfide bond.
Further Embodiment 62. The polymer nanocapsule of any one of further embodiments 60-61, wherein the at least one targeting moiety is an antibody.
Further Embodiment 63. The polymer nanocapsule of any one of further embodiments 60-62, wherein the at least one targeting moiety facilitates delivery of the polymer nanocapsule to a specific cell type, wherein the cell type is selected from the group comprising immune cells, blood cells, cardiac cells, lung cells, optic cells, liver cells, kidney cells, brain cells, cells of the central nervous system, cells of the peripheral nervous system, cancer cells, cells infected with viruses, stem cells, skin cells, intestinal cells, and/or auditory cells.
Further Embodiment 64. The polymer nanocapsule of further embodiment 63, wherein the cancer cells are cells selected from the group comprising lymphoma cells, solid tumor cells, leukemia cells, bladder cancer cells, breast cancer cells, colon cancer cells, rectal cancer cells, endometrial cancer cells, kidney cancer cells, lung cancer cells, melanoma cells, pancreatic cancer cells, prostate cancer cells, and thyroid cancer cells.
Further Embodiment 65. The polymer nanocapsule of any one of further embodiments 56-64, further comprising at least one stabilizing moiety.
Further Embodiment 66. The polymer nanocapsule of further embodiment 65, wherein the at least one stabilizing moiety includes at least one repeat group selected from the group consisting of a polyethylene glycol repeat group and a polypropylene glycol repeat group.
Further Embodiment 67. The polymer nanocapsule of further embodiment 65, wherein the at least one stabilizing moiety includes at least two polyethylene glycol repeat groups.
Further Embodiment 68. The polymer nanocapsule of any one of further embodiments 56-67, wherein the ribonucleoprotein complex includes a guide RNA that targets HPRT, a gamma-globin promoter, and/or beta-globin.
Further Embodiment 69. The polymer nanocapsule of any one of further embodiments 56-68, wherein the polymer nanocapsule has a diameter ranging from between about 50 nm to about 250 nm.
Further Embodiment 70. A polymer nanocapsule comprising a polymer shell and a ribonucleoprotein complex, wherein the polymer nanocapsule includes at least one targeting moiety adapted to facilitate delivery of the ribonucleoprotein complex to a hematopoietic stem cell, wherein the at least one targeting moiety is coupled to the polymer nanocapsule through a disulfide bond, and wherein the polymer nanocapsule further includes at least one stabilizing moiety having a hydrophilic group.
Further Embodiment 71. The polymer nanocapsule of further embodiment 70, wherein the ribonucleoprotein complex includes a guide RNA that targets HPRT, a gamma-globin promoter, and/or beta-globin.
Further Embodiment 72. The polymer nanocapsule of any one of further embodiments 70-71, wherein the at least one targeting moiety comprises an antibody.
Further Embodiment 73. The polymer nanocapsule of any one of further embodiments 70-72, wherein the hydrophilic group of the at least one stabilizing moiety comprises a polyethylene glycol group.
Further Embodiment 74. The polymer nanocapsule of any one of further embodiments 70-72, wherein the hydrophilic group of the at least one stabilizing moiety is a polypropylene glycol repeat group.
Further Embodiment 75. The polymer nanocapsule of any one of further embodiments 70-74, wherein the polymer shell comprises at least one positively charged monomer selected from the group consisting of N-(3-((4-((3-aminopropyl)amino)butyl)amino) propyl)acrylamide, N-(3-((4-((3-aminopropyl)amino)butyl)amino) propyl)methacrylamide, N-(3-((4-aminobutyl)amino)propyl)acrylamide, N-(3-((4-aminobutyl)amino)propyl)methacrylamide, N-(2-((2-aminoethyl)(methyl)amino)ethyl)acrylamide, N-(2-((2-aminoethyl)(methyl)amino)ethyl)methacrylamide, N-(piperazin-1-ylmethyl)acrylamide, N-(piperazin-1-ylmethyl)methacrylamide, N-(2-(bis(2-aminoethyl)amino)ethyl)acrylamide, N-(2-(bis(2-aminoethyl)amino)ethyl)methacrylamide, N-(3-Aminopropyl) methacrylamide hydrochloride, 2-(dimethylamino)ethyl acrylate, Dimethylamino ethyl methacrylate, (3-Acrylamidopropyl) trimethylammonium hydrochloride, and 2-aminoethyl methacrylate.
Further Embodiment 76. The polymer nanocapsule of any one of further embodiments 70-75, wherein the polymer shell further comprises a neutral monomer and a crosslinker.
Further Embodiment 77. A pharmaceutical composition comprising the polymer nanocapsule of any one of further embodiments 56-76 and a pharmaceutically acceptable carrier or excipient.
Further Embodiment 78. A modified hematopoietic stem cell prepared by contacting a hematopoietic stem cell with the pharmaceutical composition of further embodiment 77.
Further Embodiment 79. A method of treating a human patient comprising administering a therapeutically effective amount of the modified hematopoietic stem cells of further embodiment 78 to the human patient.
Further Embodiment 80. A conjugate prepared according to a process comprising reacting a derivatized polymer nanocapsule with at least one derivatized targeting moiety, the derivatized polymer nanocapsule including at least one first reactive functional group capable of participating in a click chemistry reaction, the polymer nanocapsule having a polymer shell having at least one positively charged monomer, at least one neutral monomer, and a crosslinker; and wherein the at least one derivatized targeting moiety includes a second reactive functional group capable of participating in a click chemistry reaction with the first reactive functional group.
Further Embodiment 81. The conjugate of further embodiment 80, further comprising reacting the derivatized polymer nanocapsule with at least one derivatized stabilizing moiety, the at least one derivatized stabilizing moiety including a third reactive functional group capable of participating in a click chemistry reaction with the first reactive functional group.
Further Embodiment 82. A method of treating a genetic condition within a human patient comprising:
   (i) generating a population of modified human hematopoietic stem cells by contacting an unmodified population of human hematopoietic stem cells with a polymer nanocapsule, the polymer nanocapsule including a polymer shell and a ribonucleoprotein complex, wherein the polymer shell comprises at least one positively charged monomer, at least one neutral monomer, and a crosslinker; and wherein the ribonucleoprotein complex comprises a Cas protein and a guide RNA that targets beta-globin; and
   (ii) administering the population of modified human hematopoietic stem cells to the human patient.
Further Embodiment 83. The method of further embodiment 82, wherein the ribonucleoprotein complex comprises an endonuclease and a guide RNA.
Further Embodiment 84. The method of further embodiment 83, wherein the endonuclease is a Cas protein.
Further Embodiment 85. The method of further embodiment 84, wherein the Cas protein is selected from the group consisting of Cas9 and Cas12a.
Further Embodiment 86. A conjugate comprising: (i) a polymer nanocapsule, wherein the polymer nanocapsule comprises a polymer shell and a ribonucleoprotein complex, wherein the polymer shell comprises at least one positively charged monomer, at least one neutral monomer, and a crosslinker; and (ii) at least one of (a) a targeting moiety, or (b) a stabilizing moiety coupled to the polymer nanocapsule.
Further Embodiment 87. The conjugate of further embodiment 86, wherein the at least one of the targeting moiety or the stabilizing moiety is coupled to the polymer nanocapsule through at least one linker.
Further Embodiment 88. The conjugate of further embodiment 87, wherein the at least one linker comprises a cleavable moiety.
Further Embodiment 89. The conjugate of further embodiment 87, wherein the at least one linker comprises one or more PEG groups.
Further Embodiment 90. The conjugate of further embodiment 86, wherein the conjugate comprises between 1 and 16 targeting moieties.
Further Embodiment 91. The conjugate of further embodiment 90, wherein the targeting moieties are selected from the group consisting of antibodies, peptides, lectins, transferrins, polysaccharides, nucleic acids, and aptamers.
Further Embodiment 92. The conjugate of further embodiment 91, wherein the targeting moieties are transferrins.
Further Embodiment 93. The conjugate of further embodiment 91, wherein the targeting moieties are antibodies.
Further Embodiment 94. The conjugate of further embodiment 93, wherein the antibodies are anti-cluster of differentiation marker antibodies.
Further Embodiment 95. The conjugate of further embodiment 94, wherein the anti-cluster of differentiation marker antibodies are selected from the group consisting of anti-CD3 antibodies, anti-CD4 antibodies, anti-CD8 antibodies, anti-CD34 antibodies, anti-CD45 antibodies, anti-CD133 antibodies, and combinations thereof.
Further Embodiment 96. The conjugate of further embodiment 86, wherein the conjugate comprises between 1 and 16 stabilizing moieties.
Further Embodiment 97. The conjugate of further embodiment 96, wherein the stabilizing moieties comprise one or more PEG groups.
Further Embodiment 98. The conjugate of further embodiment 96, wherein the stabilizing moieties comprise one or more PPG groups.
Further Embodiment 99. The conjugate of further embodiment 86, wherein the conjugate comprises one or more targeting moieties and one or more stabilizing moieties.
Further Embodiment 100. The conjugate of further embodiment 86, wherein the ribonucleoprotein complex comprises an endonuclease and a guide RNA.
Further Embodiment 101. The conjugate of further embodiment 100, wherein the endonuclease is a Cas protein.
Further Embodiment 102. The conjugate of further embodiment 101, wherein the Cas protein is selected from the group consisting of Cas9 and Cas12a.
Further Embodiment 103. The conjugate of further embodiment 100, wherein the guide RNA targets the HPRT locus.
Further Embodiment 104. The conjugate of further embodiment 103, wherein the guide RNA that targets HPRT comprises a nucleic acid sequence having at least 90% identity to that of SEQ ID NO: 2.
Further Embodiment 105. The conjugate of further embodiment 100, wherein the guide RNA targets the beta-globin locus.
Further Embodiment 106. The conjugate of further embodiment 105, wherein the guide RNA that targets beta-globin comprises a nucleic acid sequence having at least 90% identity to that of SEQ ID NO: 3.
Further Embodiment 107. The conjugate of further embodiment 86, wherein the at least one positively charged monomer is selected from the group consisting of:
N-(3-((4-((3-aminopropyl)amino)butyl)amino) propyl)acrylamide,
N-(3-((4-((3-aminopropyl)amino)butyl)amino)propyl)methacrylamide,
N-(3-((4-aminobutyl)amino)propyl)acrylamide,
N-(3-((4-aminobutyl)amino)propyl)methacrylamide,
N-(2-((2-aminoethylxmethyl)amino)ethyl)acrylamide,
N-(2-((2-aminoethyl)methyl)amino)ethyl)methacrylamide,
N-(piperazin-1-ylmethyl)acrylamide,
N-(piperazin-1-ylmethyl)methacrylamide,
N-(2-(bis(2-aminoethyl)amino)ethyl)acrylamide,
N-(2-(bis(2-minoethyl)amino)ethyl)methacrylamide,
N-(3-Aminopropyl)methacrylamidehydrochloride,
2-(dimethylamino)ethyl acrylate,
Dimethylamino ethyl methacrylate,
(3-Acrylamidopropyl) trimethylammonium hydrochloride,
2-aminoethyl methacrylate,
3-(Dimethylamino)propyl acrylate,
N-[3-(Dimethylamino)propyl]methacrylamide, and
any combination thereof.
Further Embodiment 108. The conjugate of further embodiment 86, wherein the neutral monomer is selected from N-(1,3-dihydroxy-2-(hydroxymethyl)propan-2-yl)acrylamide, acrylamide, N-(hydroxymethyl)acrylamide, 2-hydroxyethyl acrylate, and 2-hydroxyethyl methacrylate.
Further Embodiment 109. The conjugate of further embodiment 86, wherein the polymer shell is free of imidazolyl acryloyl monomers.
Further Embodiment 110. A conjugate comprising: (i) a polymer nanocapsule, wherein the polymer nanocapsule comprises a polymer shell and a payload, wherein the polymer shell comprises at least one positively charged monomer, at least one neutral monomer, and a crosslinker; and wherein the payload is selected from the group consisting of ribonucleoprotein complexes, siRNA molecules, shRNA molecules, expression vectors, polynucleotides, such as polynucleotides having between 50 and 500 base pairs, peptides, enzymes, antibodies, and antibody fragments; and (ii) at least one of (a) a targeting moiety, or (b) a stabilizing moiety coupled to the polymer nanocapsule.
Further Embodiment 111. The conjugate of further embodiment 110, wherein the at least one positively charged monomer is selected from the group consisting of:
N-(3-((4-((3-aminopropyl)amino)butyl)amino) propyl)acrylamide,
N-(3-((4-((3-aminopropyl)amino)butyl)amino)propyl)methacrylamide,
N-(3-((4-aminobutyl)amino)propyl)acrylamide,
N-(3-((4-aminobutyl)amino)propyl)methacrylamide,
N-(2-((2-aminoethylxmethyl)amino)ethyl)acrylamide,
N-(2-((2-aminoethylxmethyl)amino)ethyl)methacrylamide,
N-(piperazin-1-ylmethyl)acrylamide,
N-(piperazin-1-ylmethyl)methacrylamide,
N-(2-(bis(2-aminoethyl)amino)ethyl)acrylamide,
N-(2-(bis(2-aminoethyl)amino)ethyl)methacrylamide,
N-(3-Aminopropyl)methacrylamidehydrochloride,
2-(dimethylamino)ethyl acrylate,
Dimethylamino ethyl methacrylate,
(3-Acrylamidopropyl) trimethylammonium hydrochloride,
2-aminoethyl methacrylate,
3-(Dimethylamino)propyl acrylate,
N-[3-(Dimethylamino)propyl]methacrylamide, and
any combination thereof.
Further Embodiment 112. The conjugate of further embodiment 110, wherein the neutral monomer is selected from N-(1,3-dihydroxy-2-(hydroxymethyl)propan-2-yl)acrylamide, acrylamide, N-(hydroxymethyl)acrylamide, 2-hydroxyethyl acrylate, and 2-hydroxyethyl methacrylate.
Further Embodiment 113. The conjugate of further embodiment 110, wherein the conjugate comprises between 1 and 16 targeting moieties selected from the group consisting of antibodies, peptides, lectins, transferrins, polysaccharides, nucleic acids, and aptamers.
Further Embodiment 114. The conjugate of further embodiment 110, wherein the conjugate comprises between 1 and 16 targeting moieties selected from the group consisting of antibodies and transferrins.
Further Embodiment 115. A polymer nanocapsule comprising a polymer shell and a ribonucleoprotein complex, wherein the polymer shell comprises at least one positively charged monomer, at least one neutral monomer, and a crosslinker, wherein the ribonucleoprotein complex comprises at least one gRNA, wherein the at least one gRNA has at least 90% sequence identity to any one of SEQ ID NOS: 1-22 and 39-54.
Further Embodiment 116. The polymer nanocapsule of further embodiment 115, wherein the gRNA has at least 95% sequence identity to any one of SEQ ID NOS: 1-22 and 39-54.
Further Embodiment 117. A polymer nanocapsule comprising a polymer shell and a ribonucleoprotein complex, wherein the polymer shell comprises at least one positively charged monomer, at least one neutral monomer, and a crosslinker, wherein the ribonucleoprotein complex comprises at least one gRNA, wherein the at least one gRNA comprises any one of SEQ ID NOS: 1-22 and 39-54.
Further Embodiment 118. A polymer nanocapsule comprising a polymer shell and a ribonucleoprotein complex, wherein the polymer shell comprises at least one positively charged monomer, at least one neutral monomer, and a crosslinker, wherein the ribonucleoprotein complex comprises at least one gRNA, wherein the at least one gRNA targets a nucleic acid sequence having at least 90% identity to any one of SEQ ID NOS: 24-37.

All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary, to employ concepts of the various patents, applications, and publications to provide yet further embodiments.

Although the present disclosure has been described with reference to a number of illustrative embodiments, it should be understood that numerous other modifications and embodiments can be devised by those skilled in the art that will fall within the spirit and scope of the principles of this disclosure. More particularly, reasonable variations and modifications are possible in the component parts and/or arrangements of the subject combination arrangement within the scope of the foregoing disclosure, the drawings, and the appended claims without departing from the spirit of the disclosure. In addition to variations and modifications in the component parts and/or arrangements, alternative uses will also be apparent to those skilled in the art.

1. A polymer nanocapsule comprising a polymer shell and a ribonucleoprotein complex, wherein the polymer shell comprises at least one positively charged monomer, at least one neutral monomer, and a crosslinker.

2. The polymer nanocapsule of claim 1, wherein the ribonucleoprotein complex comprises an endonuclease and a guide RNA.

3. The polymer of claim 2, wherein the endonuclease is a Cas protein.

4. The polymer of claim 3, wherein the Cas protein is a Type II Cas protein or a Type V Cas protein.

5. The polymer nanocapsule of claim 3, wherein the Cas protein is selected from the group consisting of a Cas9 protein and a Cas12a protein.

6. The polymer nanocapsule of claim 2, wherein the guide RNA targets an HPRT locus, a beta-globin locus, or a CCR5 locus.

7. The polymer nanocapsule of claim 2, wherein the guide RNA comprises a nucleic acid sequence having at least 90% identity to that of SEQ ID NO: 1, SEQ ID NO: 2, or SEQ ID: 3.

8. The polymer nanocapsule of claim 1, further comprising at least one targeting moiety.

9. The polymer nanocapsule of claim 8, wherein the at least one targeting moiety is an antibody.

10. The polymer nanocapsule of claim 1, further comprising at least one stabilizing moiety.

11. The polymer nanocapsule of claim 10, wherein the at least one stabilizing moiety comprises at least one polyethylene glycol group.

12. The polymer nanocapsule of claim 1, wherein the polymer shell comprises at least two positively charged monomers.

13. A polymer nanocapsule comprising a polymer shell and a ribonucleoprotein complex, wherein the polymer nanocapsule includes at least one targeting moiety adapted to facilitate delivery of the ribonucleoprotein complex to a hematopoietic stem cell, wherein the at least one targeting moiety is coupled to the polymer nanocapsule through a disulfide bond, and wherein the polymer nanocapsule further includes at least one stabilizing moiety having a hydrophilic group.

14. The polymer nanocapsule of claim 13, wherein the ribonucleoprotein complex includes a guide RNA that targets HPRT, a gamma-globin promoter, and/or beta-globin.

15. The polymer nanocapsule of claim 13, wherein the at least one targeting moiety comprises an antibody.

16. The polymer nanocapsule of claim 13, wherein the hydrophilic group of the at least one stabilizing moiety comprises a polyethylene glycol group.

17. The polymer nanocapsule of claim 13, wherein the hydrophilic group of the at least one stabilizing moiety comprises a polypropylene glycol repeat group.

18. The polymer nanocapsule of claim 13, wherein the polymer shell further comprises a neutral monomer and a crosslinker.

19. The polymer nanocapsule of claim 18, wherein the neutral monomer is selected from the group consisting of:
N-(1,3-dihydroxy-2-(hydroxymethyl)propan-2-yl)acrylamide,
acrylamide,
N-(hydroxymethyl) acrylamide,
2-hydroxy ethyl acrylate, and
2-hydroxy ethyl methacrylate.

20. The polymer nanocapsule of claim 18, wherein the crosslinker is selected from the group consisting of:
1,3-glycerol dimethacrylate,
N,N′-methylene bisacrylamide, and
Glycerol-1,3-diglycerolate diacrylate.

21. The polymer nanocapsule of claim 13, wherein the polymer shell comprises at least one positively charged monomer selected from the group consisting of:
N-(3-((4-((3-aminopropyl)amino)butyl)amino) propyl)acrylamide,
N-(3-((4-((3-aminopropyl)amino)butyl)amino) propyl)methacrylamide,
N-(3-((4-aminobutyl)amino)propyl)acrylamide,
N-(3-((4-aminobutyl)amino)propyl)methacrylamide,
N-(2-((2-aminoethyl)(methyl)amino)ethyl)acrylamide,
N-(2-((2-aminoethyl)(methyl)amino)ethyl)methacrylamide,
N-(piperazin-1-ylmethyl)acrylamide,
N-(piperazin-1-ylmethyl)methacrylamide,
N-(2-(bis(2-aminoethyl)amino)ethyl)acrylamide,
N-(2-(bis(2-aminoethyl)amino)ethyl)methacrylamide,
N-(3-Aminopropyl) methacrylamide hydrochloride,
2-(dimethylamino)ethyl acrylate,
Dimethylamino ethyl methacrylate,
(3-Acrylamidopropyl) trimethylammonium hydrochloride,
2-aminoethyl methacrylate,
3-(Dimethylamino)propyl acrylate, and
N-[3-(Dimethylamino)propyl]methacrylamide.

22. A composition comprising the polymer nanocapsule of claim 1, and a pharmaceutically acceptable carrier or excipient.

23. A modified host cell prepared by contacting an unmodified host cell with the polymer nanocapsules of claim 1.

24. The modified host cell of claim 23, wherein the unmodified host cell is a hematopoietic stem cell.

25. The modified host cell of claim 24, wherein the hematopoietic stem cell is a CD34+ hematopoietic stem cell.

26. The modified host cell of claim 24, wherein the hematopoietic stem cell is selected from the group consisting of an allogenic hematopoietic stem cell, an autologous hematopoietic stem cell, and a sibling matched hematopoietic stem cell.

27. The modified host cell of claim 23, wherein the ribonucleoprotein complex of the polymer nanocapsule comprises at least one guide RNA having at least 95% sequence identity to any one of SEQ ID NOS: 1-23 and 39-54.

28. The modified host cell of claim 23, wherein the ribonucleoprotein complex of the polymer nanocapsule comprises at least one guide comprising any one of SEQ ID NOS: 1-23 and 39-54.

29. The modified host cell of claim 23, wherein the ribonucleoprotein complex of the polymer nanocapsule comprises at least one guide RNA targeting a sequence having at least 95% identity to any one of SEQ ID NOS: 24-38 and 55-56.

30. A method of treating a genetic condition in a human subject comprising administering to the human subject a therapeutically effective amount the modified host cells of claim 23.
